(19) United States
(12) Patent Application Publication
(10) Pub. No.: US 2026/0167703 A1
(43) Pub. Date: Jun. 18, 2026

(54) A METHOD FOR DETERMINING THE INFECTIVITY OF A VIRUS

(71) Applicant: Takeda Vaccines, Inc., Cambridge, MA (US)

(72) Inventors: Jill Livengood, Cambridge, MA (US); James Gifford, Cambridge, MA (US); Andrea Bertolotti-Ciarlet, Cambridge, MA (US); Sandra Catalina Bustamante-Lopez, Cambridge, MA (US); Isamu Tsuji, Yamaguchi (JP); Heather Watkins, Cambridge, MA (US); David Dominguez, Cambridge, MA (US); Brooke Norwood, Cambridge, MA (US)

(73) Assignee: Takeda Vaccines, Inc., Cambridge, MA (US)

(21) Appl. No.: 19/126,805

(22) PCT Filed: Nov. 1, 2023
(86) PCT No.: PCT/US2023/078311

§ 371 Date: May 2, 2025

(30) Foreign Application Priority Data
Nov. 2, 2022 (EP) 22205025.4

(51) Int. Cl. C07K 16/116; A61P 31/14; G01N 33/50; G01N 33/569

(52) U.S. Cl. CPC C07K 16/116; A61P 31/14; G01N 33/5091; G01N 33/56983; C07K 2317/76; C07K 2317/92; G01N 2333/185; G01N 2469/10

(57) ABSTRACT

The present invention relates to a method for determining the infectivity of a virus, in particular a flavivirus. Further, a method for determining the titer of a virus-specific antibody in a sample is provided. In addition, the use of the method in the quality control of vaccines and in the diagnosis of an infection are provided as well. The present invention also provides a method for determining the proportion of infectious virus particles in a sample comprising infectious and non-infectious virus particles. The present invention further relates to a maturation assay for determining the average maturation degree. The present invention also relates to novel anti-dengue virus serotype 2 (DENV2) and anti-dengue virus serotype 4 (DENV4) antibodies and antigen binding fragments thereof being useful in the method of the invention. Further, nucleic acids encoding them and host cells comprising them are provided. In addition, the use of the antibodies in the prevention or treatment of dengue disease is provided. Also, diagnostic methods using them and kits comprising them are provided.

### CO-FILED SEQUENCE LISTING

The present specification makes reference to a sequence listing (submitted electronically on the same date as the present application). The entire contents of the Sequence Listing are incorporated herein by reference.

### TECHNICAL FIELD

The present invention relates to a method for determining the infectivity of a virus, in particular a flavivirus. Further, a method for determining the titer of a virus-specific antibody in a sample is provided. In addition, the use of the method in the quality control of vaccines and in the diagnosis of an infection are provided as well. The present invention also provides a method for determining the proportion of infectious virus particles in a sample comprising infectious and non-infectious virus particles. The present invention further relates to a maturation assay for determining the average maturation degree of a flavivirus-containing sample. The present invention also relates to novel anti-dengue virus serotype 2 (DENV2) and anti-dengue virus serotype 4 (DENV4) antibodies and antigen binding fragments thereof being useful in the method of the invention. Further, nucleic acids encoding them and host cells comprising them are provided. In addition, the use of the antibodies in the prevention or treatment of dengue disease is provided. Also, diagnostic methods using them and kits comprising them are provided.

### BACKGROUND OF THE INVENTION

Vaccines for protection against viral infections have been effectively used to reduce the incidence of human disease. One of the most successful technologies for viral vaccines is to immunize animals or humans with a weakened or attenuated virus strain (a “live attenuated virus”). The limited viral replication is sufficient to express the full repertoire of viral antigens and can generate potent and long-lasting immune responses to the virus. Thus, upon subsequent exposure to a pathogenic virus strain, the immunized individual is protected from the disease. These live attenuated viral vaccines are among the most successful vaccines used in public health. Dengue disease is a mosquito-borne disease caused by infection with a dengue virus. Dengue virus infections can lead to debilitating and painful symptoms, including a sudden high fever, headaches, joint and muscle pain, nausea, vomiting and skin rashes. To date, four serotypes of dengue virus have been identified: dengue-1 (DENV-1), dengue-2 (DENV-2), dengue-3 (DENV-3) and dengue-4 (DENV-4). Dengue virus serotypes 1-4 can also cause dengue hemorrhagic fever (DHF) and dengue shock syndrome (DSS). In the most severe cases, DHF and DSS can be life threatening. Dengue viruses cause 50-100 million cases of debilitating dengue fever, 500,000 cases of DHF/DSS, and more than 20,000 deaths each year, a large portion of which are children. All four dengue virus serotypes are endemic throughout the tropical regions of the world and constitute the most significant mosquito-bome viral threat to humans there. Dengue viruses are transmitted to humans primarily by Aedes aegypti mosquitoes, but also by Aedes albopictus mosquitoes. Infection with one dengue virus serotype results in life-long protection from re-infection by that serotype, but does not prevent secondary infection by one of the other three dengue virus serotypes. In fact, previous infection with one dengue virus serotype may lead to an increased risk of severe disease (DHF/DSS) upon secondary infection with a different serotype.

Takeda has developed a tetravalent dengue vaccine candidate (TAK-003). The tetravalent dengue virus composition is a dengue virus composition comprising four different immunogenic components from the four different dengue serotypes DENV-1, DENV-2, DENV-3 and DENV-4, comprising four different live, attenuated dengue viruses, each representing one dengue serotype, and which aims to stimulate immune responses to all four dengue serotypes.

For quality control and reliable manufacture of vaccines including live attenuated viruses it is of utmost importance to determine the virus titer of the individual attenuated viruses, for example in the monovalent Bulk Drug Substance (BDS), and tetravalent vaccine drug product (DP). The determination of the virus titer can also be used as an in process control test (IPC) during manufacture.

For monovalent virus compositions or any multivalent combination thereof, the current gold standard for determining infectivity or potency is the immunofocus assay (IFA). The basic principle of the IFA is known for more than 40 years. However, the assay is laborious, technically complex and prone to operator bias due to inspection of the foci by the naked eye of a trained technician. Further, the IFA is characterized by a low throughput, because it can only be performed in a singleplex format and is currently done in 6-well plates. Thus, there is a clear need for a faster and less error prone method for determining the infectivity or potency of a virus sample which is suitable also for multiplex format.

For monovalent virus compositions or any multivalent combination thereof, there is a general need for quantification of different virus serotypes such as dengue virus serotypes in a sample. Rougemont et al., Proteomics 15 (2015), 3320-3330 describe the use of isotope dilution mass spectrometry (IDMS) for this purpose. However, this assay is expensive and requires highly trained technicians. Consequently, there is a need for a more simple and less expensive method for the quantitative determination of dengue virus serotype-specific proteins such as the envelope protein.

Anti-DENV2 and Anti-DENV4 antibodies are commercially available. However, these antibodies either do not exhibit a sufficient binding affinity and/or do not sufficiently neutralize the binding of the dengue virus to its target cell.

Therefore, there is a need for improved anti-DENV2 and anti-DENV4 antibodies having increased binding affinity and/or neutralizing activity. There is a further need for antibodies being potentially suitable in the prevention or treatment of dengue disease.

### SUMMARY OF THE INVENTION

In a first aspect the present invention provides a method for determining the infectivity of a virus in a sample comprising the steps of:
(a) seeding cells from a virus-susceptible cell line in an assay plate and culturing the cells for a culture period;
(b) preparing serial dilutions of the virus-containing sample;
(c) adding the serially diluted samples to the cells seeded and cultured in step (a) and incubating the cells over a first incubation period;
(d) culturing the cells of step (c) over a second incubation period;
(e) lysing the cells of step (d); and
(f) determining the presence and/or amount of a virus antigen of the virus in the lysate of step (e) in an immunoassay method, wherein the amount of the virus antigen is indicative for the infectivity of the virus in the sample.

In a second aspect the present invention provides the use of the method according to the present invention in the quality control of a composition comprising a monovalent virus vaccine, preferably the composition comprises a tetravalent virus vaccine composition, more preferably a tetravalent dengue virus vaccine composition.

In a third aspect the present invention provides a kit for use in the method according to the present invention comprising:
(a) at least one primary antibody specific for a virus antigen;
(b) at least one secondary antibody specific for the virus antigen, wherein the at least one secondary antibody is conjugated to a detectable label.

In a fourth aspect the present invention provides a kit for use in the method according to the present invention comprising:
(a) at least one primary antibody specific for a virus antigen;
(b) at least one secondary antibody specific for the virus antigen;
(c) a tertiary antibody specific for the constant region of the at least one secondary antibody and conjugated to a detectable label

In a fifth aspect the present invention provides a method for determining the proportion of infectious virus particles in a sample comprising infectious and non-infectious virus particles, wherein the method comprises:
(i) determining the amount of a virus antigen in the sample in an immunoassay method which is indicative for the total amount of infectious and non-infections particles in the sample;
(ii) carrying out steps (a) to (e) according to the present invention and determining the amount of the virus antigen of the virus in the lysate of step (e) in an immunoassay method, wherein the amount of the virus antigen is indicative for the amount of infectious particles in the sample prior to infection of the cells in step (c) and
(iii) calculating from the total amount of infectious and non-infectious virus particles in step (i) and the amount of infectious particles in step (ii) the proportion of the infectious particles in the virus-containing sample prior to infection of the cells in step (c).

In a sixth aspect the present invention provides an antibody specific for Dengue virus serotype 2 envelope protein (DENV2 E protein) or an antigen binding fragment thereof, wherein
(i) the VH CDR1 region of the antibody or antigen binding fragment thereof is selected from the group consisting of SEQ ID NO: 106, SEQ ID NO: 109, and SEQ ID NO: 112, or a variant thereof having at least 85% identity;
(ii) the VH CDR2 region of the antibody or binding fragment thereof is selected from the group consisting of SEQ ID NO: 107, SEQ ID NO: 110, and SEQ ID NO: 113, or a variant thereof having at least 85% identity,
(iii) the VH CDR3 region of the antibody or antigen binding fragment thereof is selected from the group consisting of SEQ ID NO: 108, SEQ ID NO: 111, and SEQ ID NO 114, or a variant thereof having at least 85% identity;
(iv) the VL CDR1 region of the antibody or antigen binding fragment thereof is selected from the group consisting of SEQ ID NO: 127, SEQ ID NO: 129, and SEQ ID NO: 131, or a variant thereof having at least 82% identity;
(v) the VL CDR2 region of the antibody or antigen binding fragment thereof is selected from the group of an amino acid sequence consisting of RAS, QAS and LAS, or a variant thereof having at least 65% identity; and
(vi) the VL CDR3 region of the antibody or antigen binding fragment thereof is selected from the group consisting of SEQ ID NO: 128, SEQ ID NO: 130, and SEQ ID NO: 132, or a variant thereof having at least 85% identity,
wherein the antibody or antigen binding fragment thereon does not cross-react with other dengue serotypes other than DENV2 and has one or more of the following properties:
(1) a binding activity for DENV2-VLP calculated as EC₅₀ value of 38 ng/ml or less; and/or
(2) a k_{off} value of 1×10⁻⁴ sec⁻¹ or less. In a seventh aspect the present invention provides an antibody specific for Dengue virus serotype 4 envelope protein (DENV4 E protein) or an antigen binding fragment thereof, wherein
(i) the VH CDR1 region of the antibody or antigen binding fragment thereof is selected from the group consisting of SEQ ID NO: 1, SEQ ID NO: 4, SEQ ID NO: 7, SEQ ID NO: 10, SEQ ID NO: 13, SEQ ID NO: 16 and SEQ ID NO: 19, or a variant thereof having at least 85% identity;
(ii) the VH CDR2 region of the antibody or binding fragment thereof is selected from the group consisting of SEQ ID NO: 2, SEQ ID NO: 5, SEQ ID NO: 8, SEQ ID NO: 11, SEQ ID NO: 14, SEQ ID NO: 17 and SEQ ID NO: 20, or a variant thereof having at least 85% identity;
(iii) the VH CDR3 region of the antibody or antigen binding fragment thereof is selected from the group consisting of SEQ ID NO: 3, SEQ ID NO: 6, SEQ ID NO: 9. SEQ ID NO: 12. SEQ ID NO: 15, SEQ ID NO: 18 and SEQ ID NO: 21, or a variant thereof having at least 85% identity;
(iv) the VL CDR1 region of the antibody or antigen binding fragment thereof is selected from the group consisting of SEQ ID NO: 50, SEQ ID NO: 52, SEQ ID NO: 54 and SEQ ID NO: 56, SEQ ID NO: 58, SEQ ID NO: 60 and SEQ ID NO: 62 or a variant thereof having at least 82% identity;
(v) the VL CDR2 region of the antibody or antigen binding fragment thereof is selected from the group of an amino acid sequence consisting of RAS. EAS and RAF, or a variant thereof having at least 65% identity; and
(vi) the VL CDR3 region of the antibody or antigen binding fragment thereof is selected from the group consisting of SEQ ID NO: 51, SEQ ID NO: 53, SEQ ID NO: 55 and SEQ ID NO: 57, SEQ ID NO: 59, SEQ ID NO: 61 and SEQ ID NO: 63, or a variant thereof having at least 85% identity,
wherein the antibody or antigen binding fragment thereof does not cross-react with dengue serotypes other than DENV4 and has one or more of the following properties:
(1) a binding activity for DENV4-VLP calculated as EC₅₀ value of 80 ng/ml or less; and/or
(2) a k_{off} value of 1×10⁻⁴ sec⁻¹ or less.

Takeda has developed a tetravalent dengue vaccine (TAK-003/“QDENGA®”). The tetravalent dengue virus composition is a dengue virus composition comprising four different immunogenic components from the four different dengue serotypes DENV-1, DENV-2, DENV-3 and DENV-4, comprising four different live, attenuated dengue viruses (designated as TDV-1, TDV-2, TDV-3 and TDV-4, respectively), each representing one dengue serotype, and which aims to stimulate immune responses to all four dengue serotypes. The amino acid sequences of TDV-1 to TDV-4 are shown in SEQ ID Nos: 1 to 4.

The present inventors have found that the claimed infectivity assay can efficiently be used to assess the infectivity of monovalent attenuated viruses such as TDV-1 to TDV-4 and multivalent drug products containing TDV-1, TDV-2, TDV-3 and TDV-4. The time to data is decreased, and the throughput is significantly higher as compared to the gold standard immunofocus assay. The assay is sensitive and serotype specific. The results of the infectivity assay are correlated with the results of the IFA.

The assay of the present invention is of particular importance to understand the content of the materials for characterizing basic aspects such as the maturation state and to support the assessment of the ratios of infectious and non-infectious particles. Large amounts of non-infectious particles may be deleterious to the product if inhibiting the immune response to infectious particles or inhibiting the internalization of infectious particles.

Further, the present assay provides high value for process development. The immunofocus assay (IFA) which is currently used to assess improvements in process development represents a bottleneck due to the relatively low throughput, its labor intensity and its long time to generate data. Thus, the infectivity of the present invention has a higher throughput, is much less labor intensive and provides data in a significantly shortened time.

By removing the infection step, testing throughput and time to data increases tremendously. The impact of manufacturing process changes on the amount of virus can be detected rapidly.

The present inventors have further found that the content assay is sensitive enough for testing of the different monovalent drug substances. The results of the content assay for determining the total amount of dengue virus protein such as the envelope protein correlate well with the results of the isotope dilution mass spectrometry. For attenuated virus-based pharmaceutical formulations it is of utmost importance not only to know the total amount of virus in the sample, but to exactly determine the proportion of actively replicating virus within the total amount of virus. This is readily apparent from the perspective of drug dosing. The combination of the content assay with the infectivity assay, the dengue envelope (E) protein content can be followed before and after infection of the cells.

The maturation assay as described herein provides a reliable and convenient method which does not require expensive instrumentation such as LC-MS.

The anti-DENV2 and anti-DENV4 antibodies described herein are highly specific for DENV2 and DENV4, respectively, with no relevant cross-reactivity to other dengue serotypes or flaviviruses other than dengue virus. Further, said antibodies improve the sensitivity of the described assays when e.g. used as capture antibody.

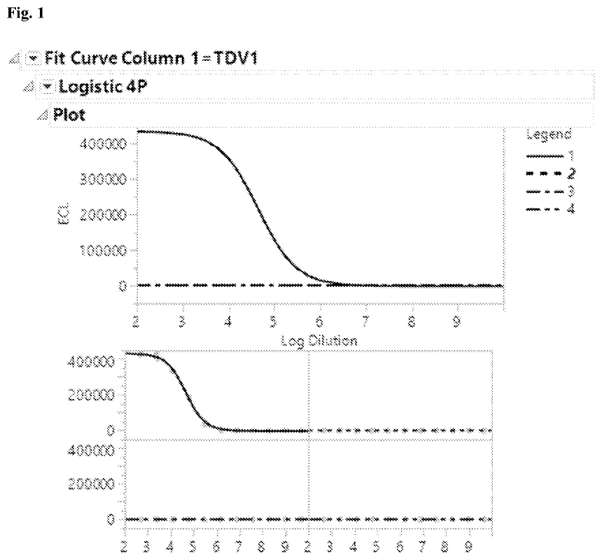

FIG. 1 shows the result of the infectivity assay for the present invention, wherein an anti-dengue serotype 1 specific antibody was used as primary antibody.

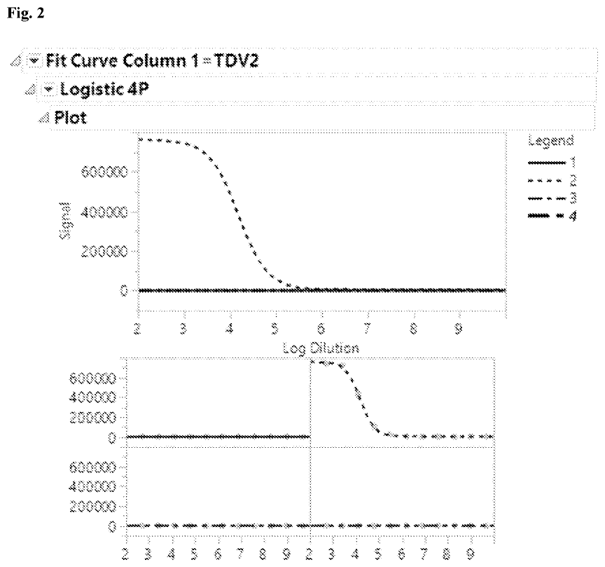

FIG. 2 shows the result of the infectivity assay for the present invention, wherein an anti-dengue serotype 2 specific antibody was used as primary antibody.

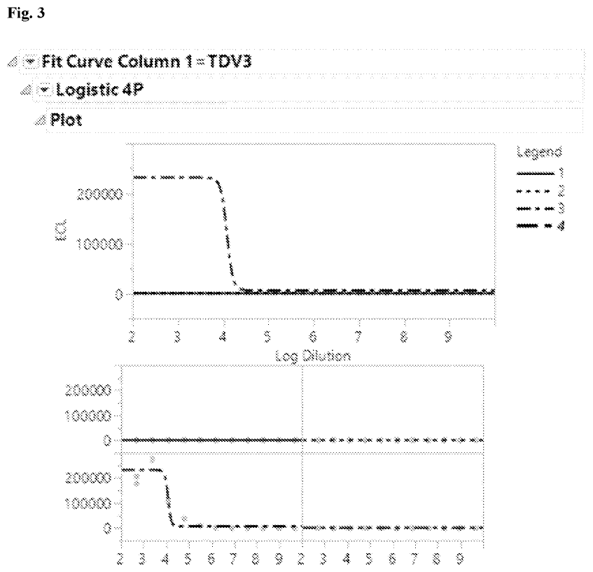

FIG. 3 shows the result of the infectivity assay for the present invention, wherein an anti-dengue serotype 3 specific antibody was used as primary antibody.

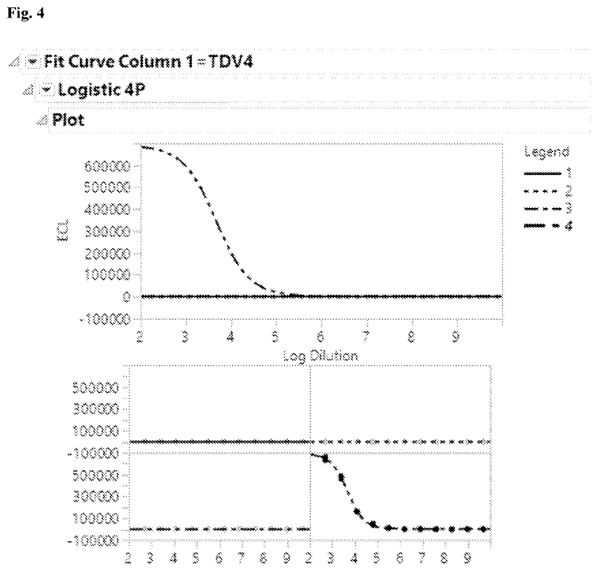

FIG. 4 shows the result of the infectivity assay for the present invention, wherein an anti-dengue serotype 4 specific antibody was used as primary antibody.

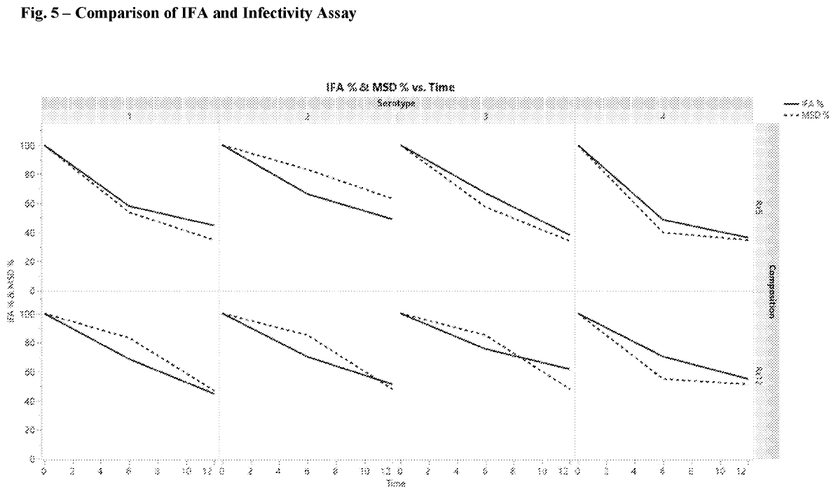

FIG. 5 shows the strong correlation of the results of the infectivity assay of the present invention with the prior art immunofocus assay.

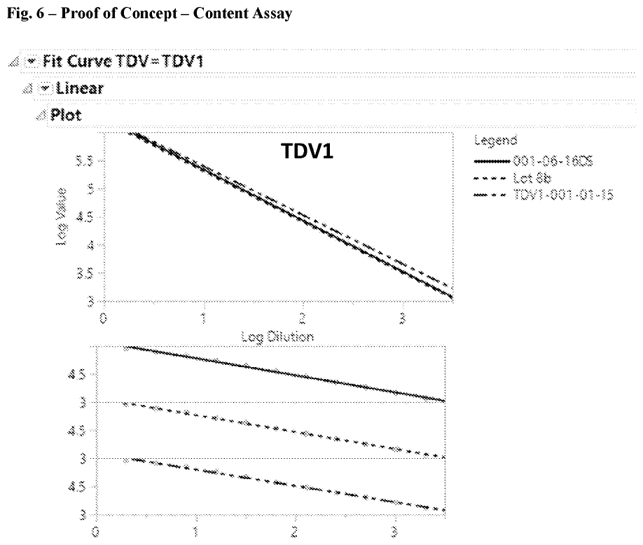

FIG. 6 shows the result of the content assay for the present invention, wherein an anti-dengue serotype 1 specific antibody was used as primary antibody.

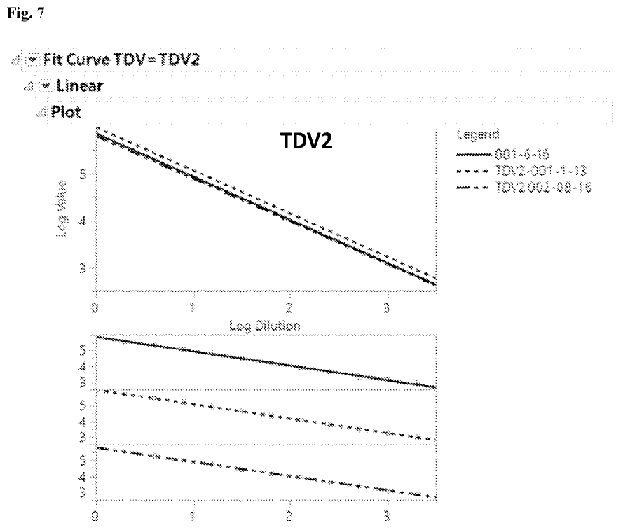

FIG. 7 shows the result of the content assay for the present invention, wherein an anti-dengue serotype 2 specific antibody was used as primary antibody.

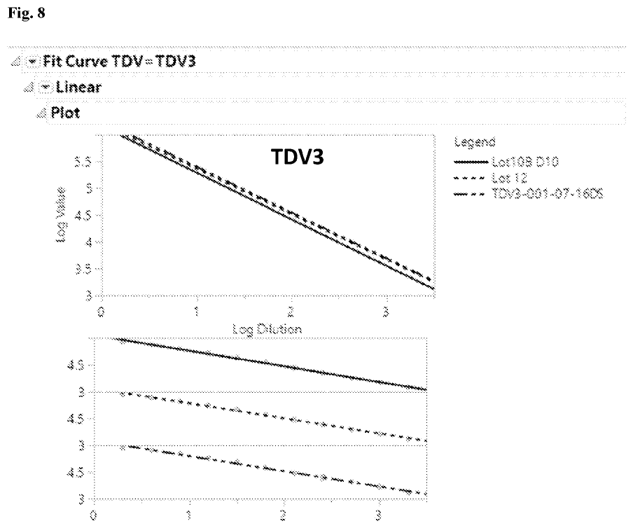

FIG. 8 shows the result of the content assay for the present invention, wherein an anti-dengue serotype 3 specific antibody was used as primary antibody.

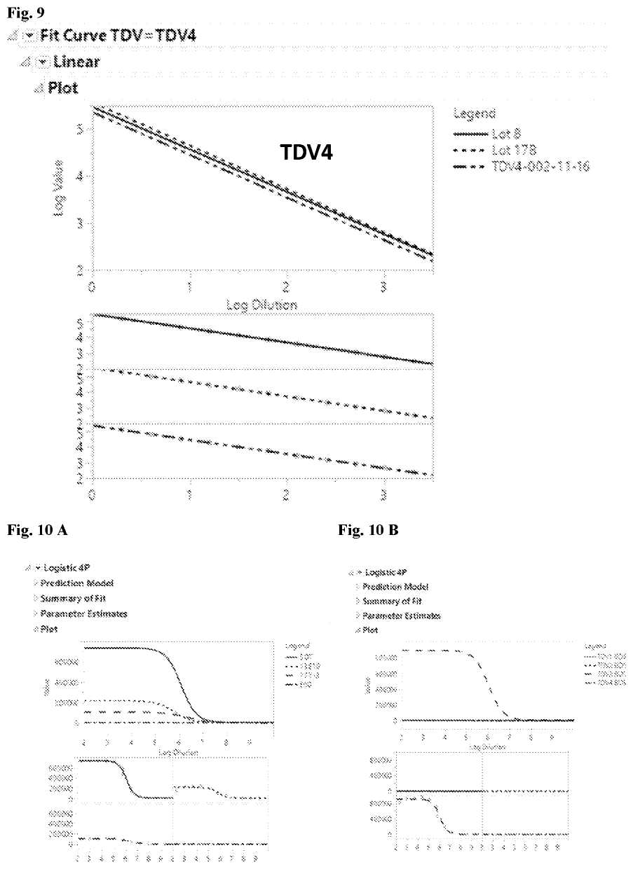

FIG. 9 shows the result of the content assay for the present invention, wherein an anti-dengue serotype 4 specific antibody was used as primary antibody.

FIGS. 10A and B show the result of the content assay using the anti-DENV3 antibodies 5D7, 13E10, 777-3 and E60 which were coated on the plates.

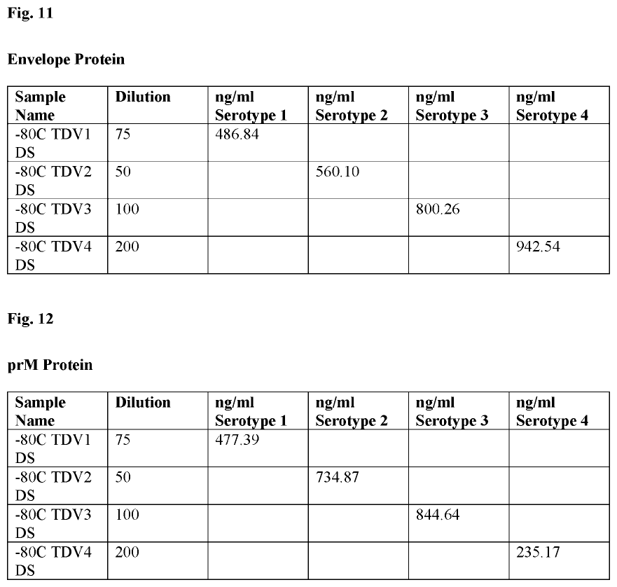

FIGS. 11 and 12 show the results for the E protein and prM protein determination in the monovalent drug substances of TDV-1 to TDV-4.

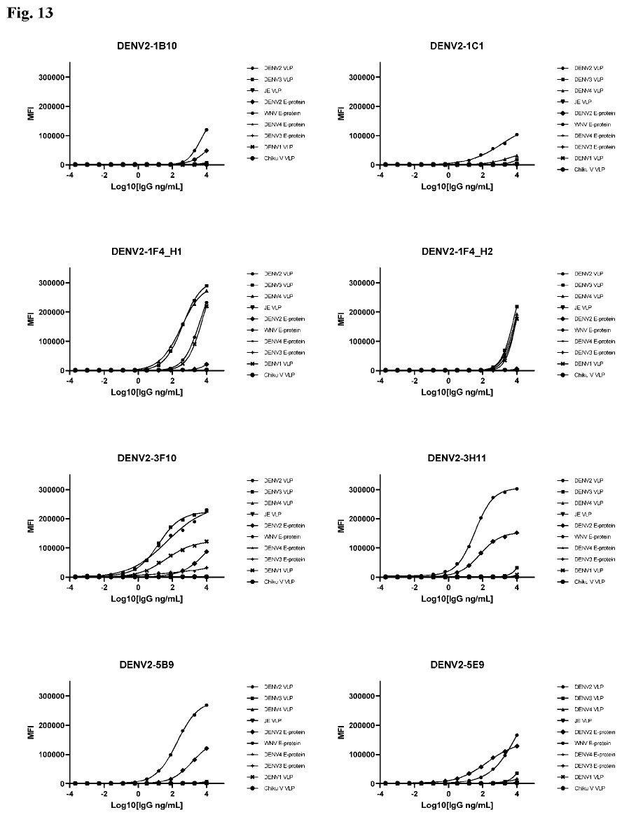

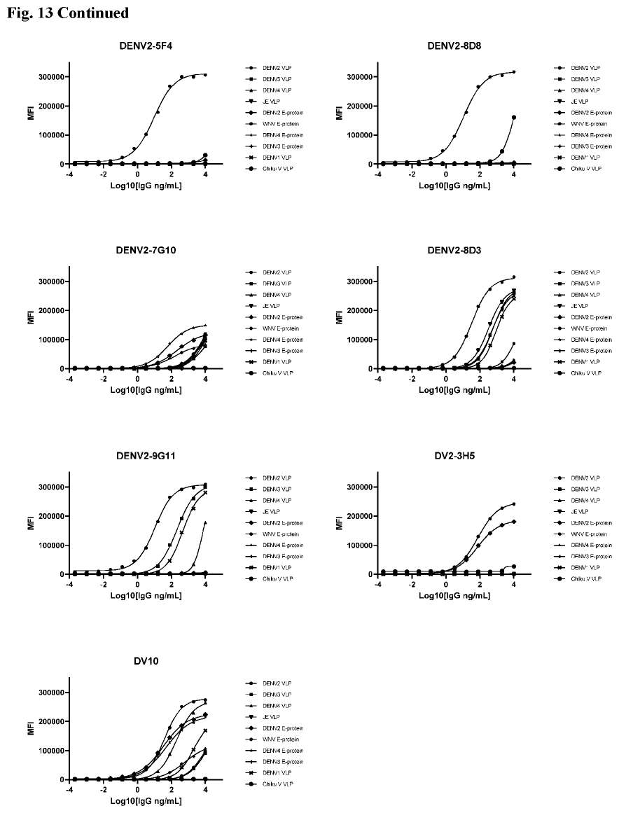

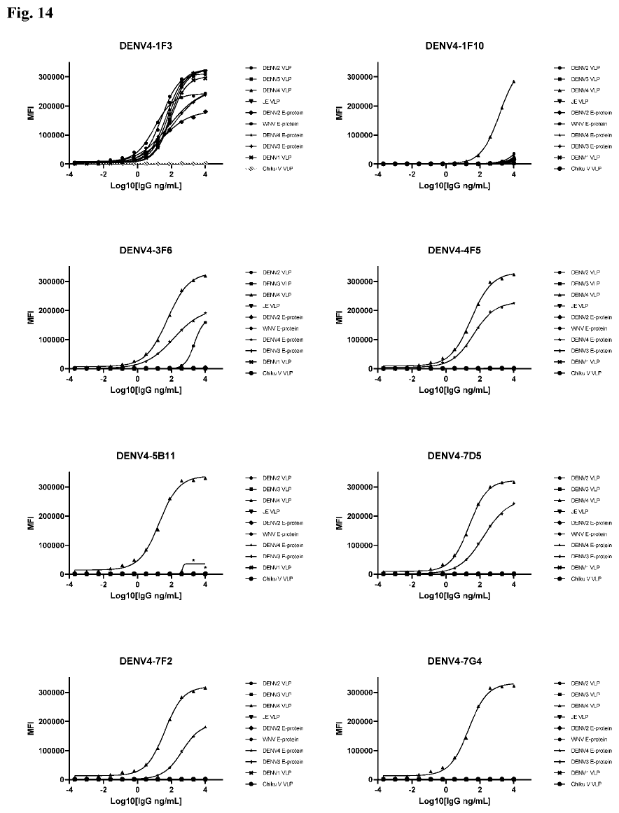

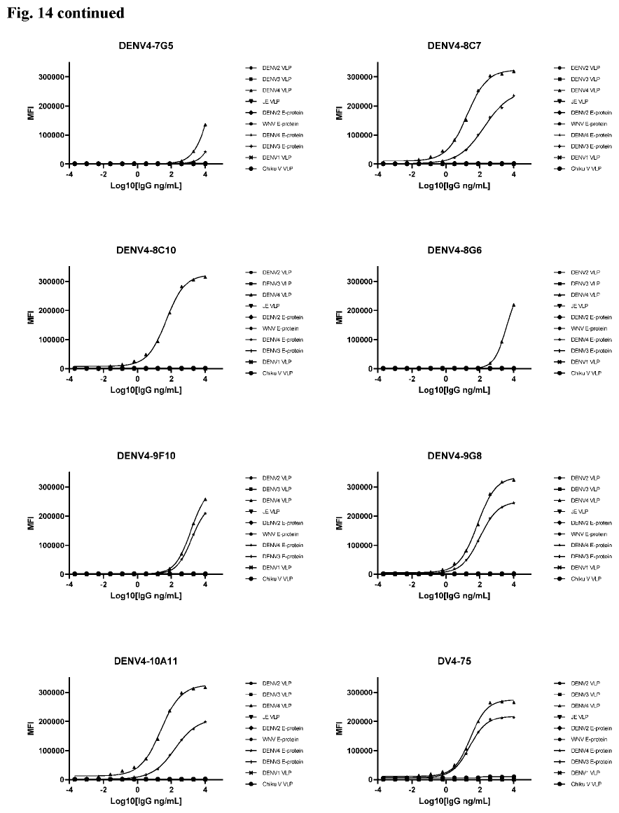

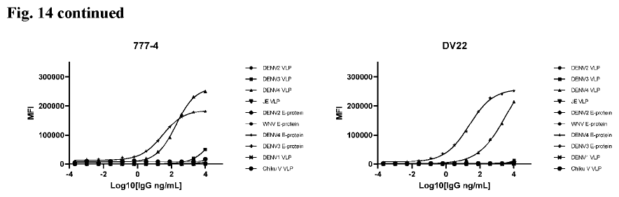

FIGS. 13 and 14 show the binding activity of anti-DENV2 and anti-DENV4 mAb measured in a Luminex assay.

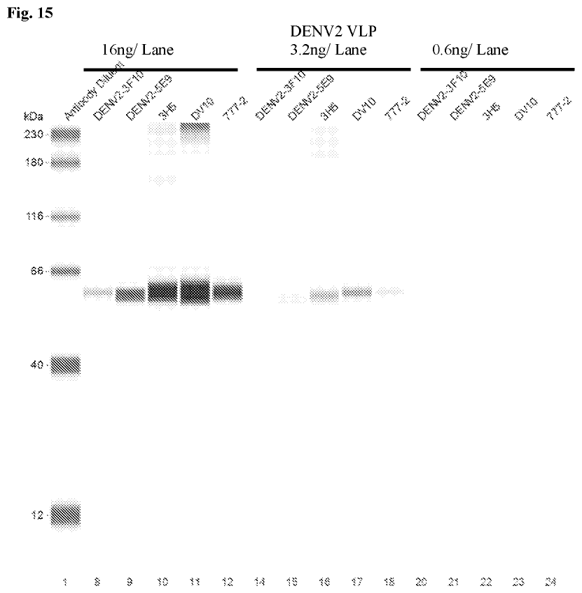

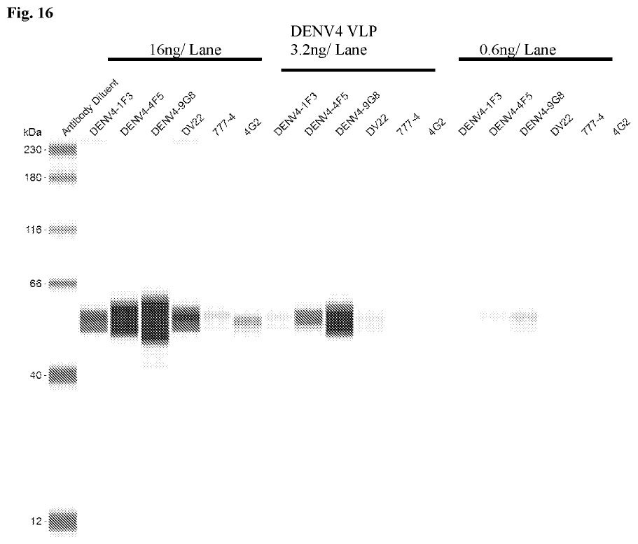

FIGS. 15 and 16 show the Western Blot reactivity of particular anti-DENV2 and anti-DENV4 clones.

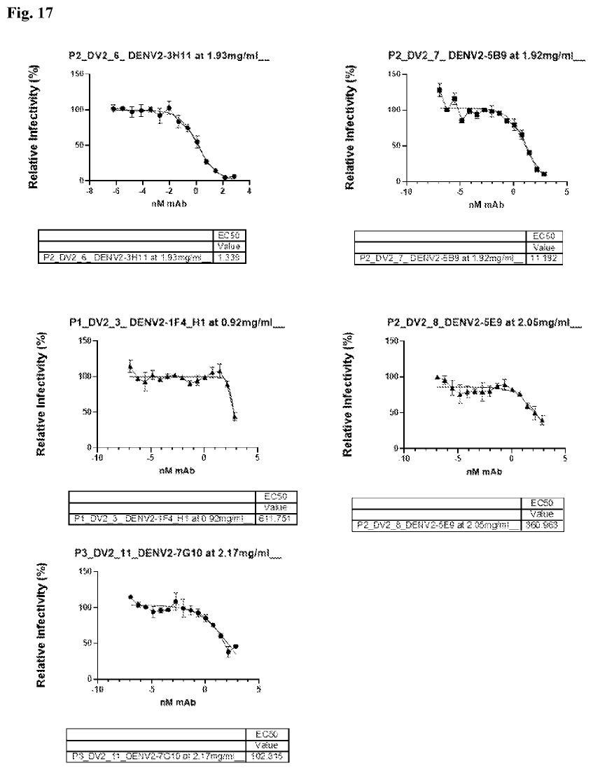

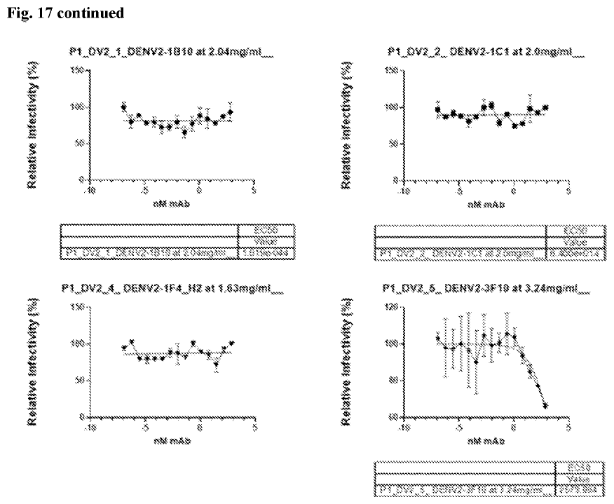

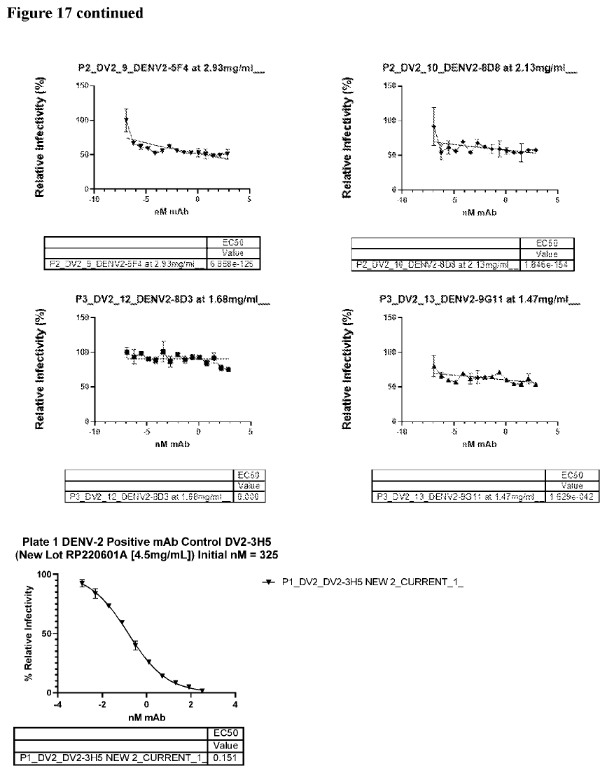

FIG. 17 shows the neutralizing activity of anti-DENV2 mAbs.

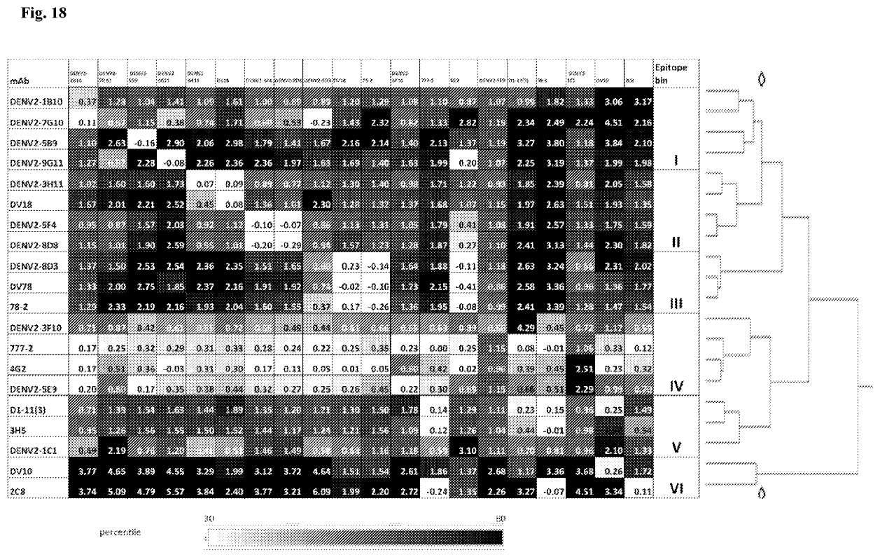

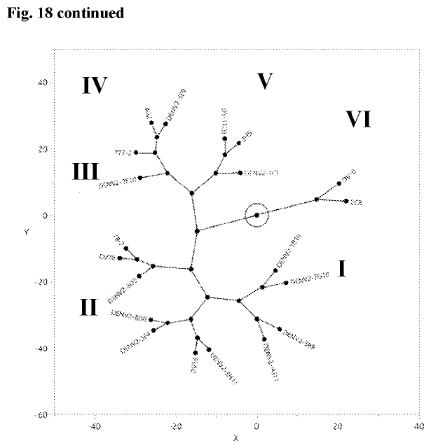

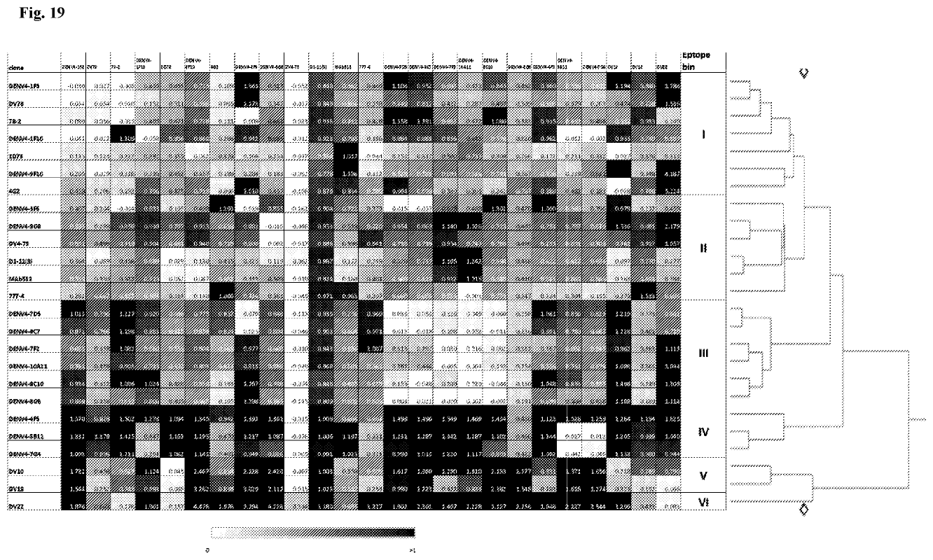

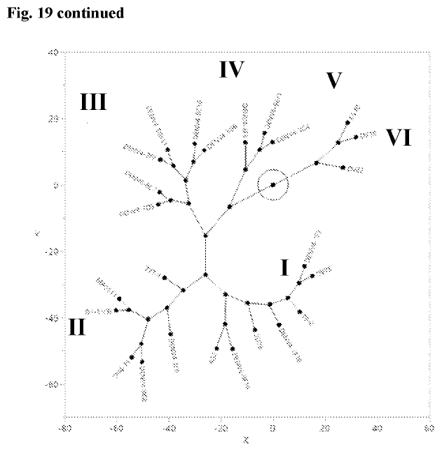

FIGS. 18 and 19 show the epitope binning results of anti-DENV2 and anti-DENV4 mAbs.

### DETAILED DESCRIPTION OF THE INVENTION

“plaque-forming virus” herein includes viruses inducing cell lysis or death upon infection of a host cell. Plaque-forming viruses include flaviviruses, picornaviruses, influenza viruses, and herpesviruses. Preferably the flavirus is selected from Yellow fever virus. Japanese encephalitis virus, dengue virus and Zika virus, more preferably, the virus is a dengue virus selected from DENV1, DENV2, DENV 3 and DENV4.

“Dengue virus” herein includes any wild-type or dengue virus mutant. The mutant may naturally occur or obtained by genetic engineering of the wild-type virus. The genetic modifications include additions, deletions, insertions and/or substitutions. The modifications are not particularly limited.

The term “dengue virus” also includes chimeras with genetic information from at least one further virus. The chimera may contain genetic information from another flavivirus such as Yellow Fever virus, Japanese Encephalitis virus, Murray Valley encephalitis virus and West Nile virus. Preferably, the chimera includes at least the E gene from another dengue virus strain.

Most preferably, the term “dengue chimera” is a dengue/dengue chimera derived from two different dengue viruses. Multivalent compositions comprising three or tetravalent compositions comprising four dengue/dengue chimeras are particularly envisaged.

The dengue virus also includes dengue virus chimera comprising more than one dengue virus subtype. Suitable variants of dengue virus chimera are described in detail in WO 2020/051328. The disclosure of which is incorporated herein by reference. Preferably, the methods according to the invention are used for determining the virus titer of the dengue virus variants TDV-1. TDV-2, TDV-3 and TDV-4 outlined below.

The term also includes dengue virus chimera comprising genetic information from another flavivirus. Preferably, the other flavivirus is selected from Japanese encephalitis virus, Tick-borne encephalitis virus, West Nile virus, Yellow fever virus and Zika virus.

“Dengue virus vaccine composition” herein includes monovalent compositions comprising only a single dengue virus or a dengue virus chimera. It also includes a flavivirus chimera comprising at least the E gene from dengue virus. The term also includes multivalent composition comprising more than one single dengue virus or chimera or flavivirus chimera comprising at least the E gene from dengue virus. Preferably, the multivalent compositions include dengue virus from more than one subtype, wherein the subtype is selected from DENV-1, DENV-2, DENV-3 and DENV-4. More preferably, the multivalent composition is a tetravalent composition comprising dengue viruses and/or chimeras from each dengue virus subtype.

The dengue virus structural envelope (E) protein and pre-membrane (prM) protein have been identified as the primary antigens that elicit a neutralizing protective antibody response. For creation of the tetravalent dengue vaccine (TDV), TDV-2 was modified by replacing the nucleic acid sequence encoding the DENV-2 prM and E glycoproteins with the nucleic acid sequence encoding the corresponding wild type prM and E glycoproteins from the DENV-1, DENV-3, and DENV-4 wild type strains DENV-1 16007, DENV-3 16562 or DENV-4 1036 virus, respectively, using standard molecular genetic engineering methods (Huang et al. (2003) J. Virol. 77(21): 11436-11447).

“Infectivity” is the capacity of viruses to enter the host cell and exploit its resources to replicate and produce progeny infectious viral particles. It is assessed as the infectious titer. Herein, the term “infectivity” also includes virus potency.

For quality control and reliable manufacture of vaccines including live attenuated viruses it is of utmost importance to determine the virus titer of the individual attenuated viruses, for example in the monovalent Bulk Drug Substance (BDS), and tetravalent vaccine drug product (DP). The determination of the virus titer can also be used as an in process control test (IPC) during manufacture.

As used herein, a “dengue-susceptible cell line” is a cell line which can be kept in culture in vitro and which can be infected with dengue virus. In the context of the present invention the dengue-susceptible cell line is capable of being lysed upon replication of the virus in the cells so that plaques are formed. Suitable dengue-susceptible cell lines include, but are not limited to, Vero cells, LLC-MK2 cells and BHK-21 cells. Preferably, the dengue-susceptible cell line is a Vero cell line.

### Step (a)

As used herein, “culturing” means maintaining cells under conditions selected such that the cells remain viable and able to divide. Such conditions include temperature, pH and culture medium. Typically, mammalian cells are cultured at a temperature of about 37° C.

The dengue-virus containing sample may be the virus containing samples as used in the culture of viruses for manufacturing purposes, as well as the bulk drug substance (BDS) containing the monovalent dengue virus serotype, mixtures of different serotypes and in particular the tetravalent final bulk drug product (BDP). The dengue-virus containing sample also includes serum samples obtained by collecting blood from a human subject and separating the serum from the other components of the blood.

### Step (b)

In the method of the present invention serial dilutions of the dengue virus-containing sample are prepared. The serial dilution of the dengue virus-containing samples is the stepwise dilution of the sample according to a given dilution factor. In one embodiment, if the dengue virus-containing sample, is a monovalent BDS, the dilution factor may be between 1:200 to 1:195,3125,000. In another embodiment, if the dengue virus-containing sample is the tetravalent dengue vaccine, the dilution factor may be between 1:2 to 1:524288.

In one embodiment, the dengue-susceptible cell line used in step (a) is selected from Vero cells, LLC-MK2 cells and BHK-21 cells. Preferably, the dengue-susceptible cell line used in step (a) is a Vero cell line.

Preferably, the dengue-susceptible cell line is seeded on 96 or 384 well plates, i.e. a defined amount of the dengue-susceptible cell line is introduced into a well of the plate which contains a suitable growth medium for the dengue-susceptible cell line. More preferably, 96 well plates are used.

Suitable growth media for dengue-susceptible cell lines are known to the skilled person and include DMEM with 10% fetal bovine serum. The dengue-susceptible cell line is preferably seeded with a density of 1 to 4×10⁵ cells per ml, more preferably of 1.5 to 3.5×10⁵ cells per ml, even more preferred of 2 to 3×10⁵ cells per ml and most preferably of 3.5×10⁵ cells per ml. In some embodiments, the dengue-susceptible cell line is cultured for a culture period of 12 to 72 hours, preferably 12 to 60 hours and most preferably of 16 to 24 hours. The culture period is calculated from the time the cells are seeded until the time the separate mixtures of the serially diluted dengue virus-containing samples are added to the cells. Preferably, the cells are grown to at least 90%, more preferably at least 95% confluence.

### Step (c)

The serially diluted dengue virus-containing samples prepared in step (b) are added to the dengue-susceptible cell line to allow for virus adsorption. The cells are incubated with the serially diluted samples for a period of 1 to 10 hours, more preferably 2 to 6 hours, even more preferably, about 4 hours at a temperature of about 37° C.

In a preferred embodiment the first incubation in step (c) is carried out in the presence of a non-ionic detergent, preferably the non-ionic detergent is a block copolymer of ethylene oxide and propylene oxide, more preferably the non-ionic detergent is F127 (also designated as Pluronic® F 127, poloxamer 407, Kolliphor® P 407 and Lutrol® F 127).

### Step (d)

In one embodiment, the second incubation period is the same for each of the different dengue serotypes. Preferably it is from about 20 to about 130 hours, more preferably from about 48 to about 96 hours, and even more preferred from about 70 to about 74 hours.

In another embodiment, different second incubation periods are used in step (d) for the different dengue serotypes. Preferably, if a monovalent sample is tested, the incubation periods for dengue serotypes may be 20 to 130 hours. More preferably, if a monovalent sample is tested, the incubation periods for dengue serotypes 1, 2 and 3 are 93±6 hours; and for dengue serotype 4 the incubation period is 117±6 hours. Further preferably, if a tetravalent sample containing each of dengue serotypes 1 to 4 is tested, the incubation periods for dengue serotypes 2 and 3 are 93±6 hours; and the incubation periods for dengue serotypes 1 and 4 are 117±6 hours.

### Step (e)

Before lysis, the media are removed. The cells of step (d) are subsequently lysed. For this purpose, a cell lysis buffer may be used which is known to the skilled person. Most lysis buffers contain buffering salts such as Tris, sodium dihydrogen phosphate/disodium hydrogen phosphate and HEPES and ionic salts such as NaCL, KCL and (NH₄)₂SO₄ to regulate the pH and osmolarity of the lysate. In some cases, detergents such as Triton X-100. CHAPS or SDS or cationic detergents are added to break up membrane structures. For lysis buffers targeted at protein extraction, protease inhibitors are often included, and some cases are almost required.

For lysation, the cells are incubated over a period of about 30 min to 4 hours, preferably from about 30 min to about 90 min with the lysis buffer. The lysate can be used immediately for the determination or frozen with later determination of the virus antigen of interest.

### Step (f)

“virus antigen” includes any fragment of a virus, in particular of proteins encoded by the virus genome. The flavivirus genome encodes a capsid protein, a pre-membrane protein, an envelope protein and the non-structural proteins NS1. NS2A, NS2B, NS3, NS4A, NS4B and NS5. Thus, for flaviviruses the antigen may be a fragment of any of the aforementioned proteins. Further included are non-naturally occurring variants such as mutants or chimeras of these protein antigens.

The “immunoassay method” of step (f) includes any immunoassay method well known to the skilled person. Preferably, the immunoassay method is selected from Enzyme-linked immunoassay (ELISA), radioimmunoassay (RIA), lateral flow immunoassay, electrochemical immunoassay, colorimetric assay, chemiluminescent immunoassay, fluorescent immunoassay, surface plasmon resonance-based immunoassay, lab-on-a-chip-based immunoassay, more preferably the immunoassay is an electrochemical, chemiluminescent, fluorescent immunoassay or an ELISA, and even more preferred an electrochemical immunoassay.

The immunoassay method may be a direct or indirect immunoassay. Preferably the immunoassay is in the format of a sandwich assay.

In a preferred embodiment the immunoassay method comprises the following steps:
(a) coating at least one well of an assay plate with at least one primary antibody specific for the virus antigen;
(b) contacting the at least one primary antibody coated on the at least one well of the assay plate of step (a) with the lysate of claim 1, step (e) to allow binding of the virus antigen to the primary antibody;
(c1) contacting the virus antigen bound to the primary antibody with at least one secondary antibody specific for the virus antigen, wherein the at least one secondary antibody is conjugated to a detectable label; or
(c2) contacting the virus antigen bound to the primary antibody with at least one secondary antibody specific for the virus antigen and further contacting the at least one secondary antibody with a tertiary antibody being specific for the constant region of the at least one secondary antibody and conjugated to a detectable label,
(d) detecting a signal from the at least one secondary antibody conjugated to the detectable label of step (c1) or from the tertiary antibody conjugated to the detectable label of step (c2), wherein the presence and/or amount of the detectable label is indicative for the infectivity of the virus in the sample.

In a preferred embodiment in the coating step (a) a first binding partner is linked to the well of the plate and a second binding partner is linked to the at least one primary antibody and the coating of the antibody to the well is effected by the formation of a binding pair of the first and second binding partner, more preferably the first and the second binding partner is selected from a specific binding pair selected from streptavidin/biotin, antigen/antibody, and sugar/lectin, even more preferred the first and second binding partner is selected from streptavidin/biotin.

The “primary antibody” is a dengue serotvpe-specific antibody which may be obtained commercially or may be prepared. The skilled person knows how to prepare serotype-specific antibodies. Suitable approaches are described for example in Gentry et al. (1982) Am. J. Trop. Med. Hyg. 31, 548-555; Henchal et al. (1985) Am. J. Trop. Med. Hyg. 34, 162-169; and Henchal et al. (1982) Am. J. Trop. Med. Hyg. 31(4):830-6). For example, mice can be immunized with a specific dengue serotype and the B cells isolated from these mice can be fused with a fusion partner to prepare a hybridoma. Suitable serotype-specific antibodies are selected based on the binding of the antibodies to the serotype with which the mice were immunized and lack of binding to those serotypes with which the mice were not immunized. In one embodiment, the mice were immunized with a serotype selected from dengue 1 strain Hawaii, Envelope, dengue 2 strain New Guinea C, Envelope, isotype 1, dengue 3 strain H87, Envelope, isotype 2A, and dengue 4 strain H241, Envelope, isotype 2A.

The “secondary antibody” is an anti-dengue antibody. The secondary antibody may be serotype-specific or reactive with more than one serotype. Preferably, the secondary antibody recognizes each of the four dengue serotypes. The secondary antibody is commercially available or can be made following methods known to the skilled person described above. It is particularly preferred to use the antibody DV-78. The secondary antibody may be directly conjugated with a detectable label. If not, a third antibody conjugated to a detectable label is used.

The “tertiary antibody” if used is specific for the Fc part of the dengue serotype specific secondary antibody. The tertiary antibody preferably is labeled with a detectable label.

The “detectable label” conjugated to an antibody may be a radioisotope, a fluorophore, a chemiluminescent label, an enzyme, a particle, a metallic ion, an enzymatic cofactor, an enzymatic substrate, a protein or an ionophore. In a preferred embodiment the detectable label is selected from an enzyme, fluorescent, chemiluminescent or an electroluminescent label, preferably an electroluminescent label. The enzyme may be HRP, G-6-PDH, MDH or NADH dehydrogenase or acetylcholinesterase. The fluorophore may be fluorescein, umbelliferone, or rhodamine. The chemiluminescent label may be luminol or a derivative thereof, or luciferase/luciferin.

Electroluminescent labels generate light when stimulated by electricity in the appropriate chemical environment. Electrochemilumescence-based immunoassays provide high sensitivity, broad dynamic range with a low background. Even more preferred, the detectable label is a sulfo-tag label (Meso Scale Discovery, Inc.).

The detectable label may be adsorbed to the antibody, but is preferably conjugated by a chemical bond.

### Singleplex

In one preferred embodiment the assay is carried out in singleplex format. To this end, the at least one primary antibody present in a first well of the assay plate consists of an antibody specific for a virus antigen from a first dengue virus serotype, and wherein the at least one primary antibody present in a second well of the assay plate consists of an antibody specific for a virus antigen from a second dengue virus serotype.

### Multiplex

In another preferred embodiment the assay is carried out in multiplex format. To this end, the at least one primary antibody present in a first well of the assay plate comprises a mixture of
(i) a primary antibody specific for a virus antigen from a first dengue virus serotype, wherein the primary antibody is provided at a first site of the well, and
(ii) a primary antibody specific for a virus antigen from a second dengue virus serotype, wherein the primary antibody is provided at a second site of the same well.

By fine printing of the different primary antibodies to different sites of the well, different antigens can be determined in parallel in the same well.

In a preferred embodiment the at least one secondary antibody is a secondary antibody specific for a virus antigen from at least one dengue virus serotype, preferably the secondary antibody binds to the dengue virus antigen from each of the four dengue virus serotypes.

In a further aspect the present invention provides the use of the inventive method in the quality control of a virus preparation or a vaccine composition. The reliable determination of the dengue virus titer is highly relevant at different stages of the vaccine manufacturing. It is used for assessing the quality of the different viral stocks. In addition, it is important for assessing the titer of the monovalent drug substance and before and after the final formulation of the tetravalent drug product.

In a further aspect the present invention provides the use of the inventive method in the diagnosis of a blood sample from a Dengue infected individual. The method can therefore be determined whether a superinfection by two different dengue serotypes is present in the individual.

The present invention also provides a kit for use in the method according to the present invention comprising:
(a) at least one primary antibody specific for a virus antigen;
(b) at least one secondary antibody specific for the virus antigen, wherein the at least one secondary antibody is conjugated to a detectable label.

The present invention also provides a kit for use in the method according to the present invention comprising:
(a) at least one primary antibody specific for a virus antigen;
(b) at least one secondary antibody specific for the virus antigen;
(c) a tertiary antibody specific for the constant region of the at least one secondary antibody and conjugated to a detectable label.

In a further aspect the present invention provides a method for determining the proportion of infectious virus particles in a sample comprising infectious and non-infectious virus particles, wherein the method comprises:
(i) determining the amount of a virus antigen in the sample in an immunoassay method which is indicative for the total amount of infectious and non-infections particles in the sample;
(ii) carrying out steps (a) to (e) of the method according to the present invention and determining the amount of the virus antigen of the virus in the lysate of step (e) in an immunoassay method, wherein the amount of the virus antigen is indicative for the amount of infectious particles in the sample prior to infection of the cells in step (c) and
(iii) calculating from the total amount of infectious and non-infectious virus particles in step (i) and the amount of infectious particles in step (ii) the proportion of the infectious particles in the virus-containing sample prior to infection of the cells in step (c).

For quality control purposes it is of utmost relevance not only to know the total amount of virus in a pharmaceutical formulation, but to know the proportion of active virus capable of infecting host cells in order to control the dosage of the pharmaceutical formulation to be administered. To solve this problem, the present invention also provides a combination of the above described method with an immunoassay method in step (i), which then allows a calculation of the amount of infectious particles and the proportion of the infectious particles in the virus-containing sample. The immunoassay method in step (i) for determining the amount of infectious and non-infectious particles in a sample is hereinafter also called the content assay.

### Content Assay

The immunoassay method of step (i) may be selected from Enzyme-linked immunoassay (ELISA), radioimmunoassay (RIA), lateral flow immunoassay, electrochemical immunoassay, colorimetric assay, chemiluminescent immunoassay, fluorescent immunoassay, surface plasmon resonance-based immunoassay, lab-on-a-chip-based immunoassay. Preferably, the immunoassay is an electrochemical, chemiluminescent, fluorescent immunoassay or an ELISA. Even more preferred, the immunoassay is an electrochemical immunoassay. The immunoassay method of step (i) may be a direct or indirect immunoassay. Preferably, the immunoassay is in the format of a sandwich assay.

In a preferred embodiment the immunoassay method of step (i) comprises:
(a) coating at least one well of an assay plate with at least one primary antibody specific for the virus antigen;
(b) contacting the at least one primary antibody coated on the at least one well of the assay plate of step (a) with the lysate of claim 1, step (e) to allow binding of the virus antigen to the primary antibody;
(c1) contacting the virus antigen bound to the primary antibody with at least one secondary antibody specific for the virus antigen, wherein the at least one secondary antibody is conjugated to a detectable label; or
(c2) contacting the virus antigen bound to the primary antibody with at least one secondary antibody specific for the virus antigen and further contacting the at least one secondary antibody with a tertiary antibody being specific for the constant region of the at least one secondary antibody and conjugated to a detectable label; and
(d) detecting a signal from the at least one secondary antibody conjugated to the detectable label of step (c1) or from the tertiary antibody conjugated to the detectable label of step (c2), wherein the presence and/or amount of the detectable label is indicative for the total amount of infectious and non-infectious particles in the sample.

The primary antibody, secondary antibody, tertiary antibody and detectable label are as defined above.

### Maturation Assay

In a further aspect the present invention provides a method for determining the average maturation degree of a population of flaviviruses in a flavivirus-containing sample, wherein the method comprises:
(a) coating at least one well of an assay plate with at least one primary antibody specific for a flavivirus envelope protein (E protein) antigen;
(b) contacting the at least one primary antibody coated on the at least one well of the assay plate of step (a) with the flavivirus-containing sample to allow binding of the virus antigen to the primary antibody;
(c1) contacting the virus antigen bound to the primary antibody with (i) a secondary antibody specific for the E protein antigen, wherein the at least one secondary antibody is conjugated to a detectable label; or
(c2) contacting the virus antigen bound to the primary antibody with the (i) secondary antibody specific for the E protein antigen and further contacting the at least one secondary antibody with a tertiary antibody being specific for the constant region of the at least one secondary antibody and conjugated to a detectable label;
(d) determining the amount of the E protein antigen in the sample;
(e) coating at least one well of an assay plate with at least one primary antibody specific for a flavivirus envelope protein (E protein) antigen;
(f) contacting the at least one primary antibody coated on the at least one well of the assay plate of step (e) with the flavivirus-containing sample to allow binding of the virus antigen to the primary antibody;
(g1) contacting the virus antigen bound to the primary antibody with (ii) a secondary antibody specific for a portion of the pr domain of the flavivirus prM protein, wherein the at least one secondary antibody is conjugated to a detectable label; or
(g2) contacting the virus antigen bound to the primary antibody with the (i) secondary antibody specific for the E protein antigen and further contacting the at least one secondary antibody with a tertiary antibody being specific for the constant region of the at least one secondary antibody and conjugated to a detectable label;
(h) determining the amount of the prM protein in the sample; and
(i) calculating the ratio of the amount of prM protein antigen to the amount of E protein which is indicative for the average maturation degree of the flaviviruses in the sample.

The higher the prM/E ratio, the higher the proportion of immature flavivirus particles in the flavivirus-containing sample.

In a preferred embodiment the detectable label is selected from an enzyme, fluorescent, chemiluminescent or an electroluminescent label, preferably an electroluminescent label, more preferably a sulfotag label.

In a further preferred embodiment the at least one primary antibody present in a first well of the assay plate consists of an antibody specific for a dengue virus envelope antigen from a first dengue virus serotype, and wherein the at least one primary antibody present in a second well of the assay plate consists of an antibody specific for a dengue virus envelope antigen from a second dengue virus serotype.

The at least one primary antibody specific a dengue flavivirus envelope protein antigen may be an antibody specific for DENV2, DENV3 or DENV4.

The primary antibody specific for DENV2 or specific for DENV4 may be an antibody according to the present invention as described below.

The primary antibody specific for DENV3 may be an anti-DENV3 antibody, preferably an antibody as described in PCT/US2023/072002, the disclosure of which is incorporated herein by reference. In particular, the anti-DENV3 antibody may be selected from the clone 8D4, 12H6, 5D7 and 13E10. The VH region of the clone 5D7 comprises the amino acid sequence of SEQ ID NO: 151 and the VL region of said clone comprises the amino acid sequence of SEQ ID NO: 152. The VH region of the clone 13E10 comprises the amino acid sequence of SEQ ID NO: 153 and the VL region of said clone comprises the amino acid sequence of SEQ ID NO: 154.

In another embodiment the present invention provides the use of the maturation assay method according to present invention in a quality control method for live, attenuated flavivirus vaccines.

In a preferred embodiment the at least one primary antibody specific for a flavivirus envelope protein (E protein) antigen is an anti-DENV2 envelope protein antibody, preferably the at least one antibody is an antibody according to the present invention.

In a further preferred embodiment the at least one primary antibody specific for a flavivirus envelope protein (E protein) antigen is an anti-DENV3 envelope protein antibody, preferably,
the VH region of the at least one antibody comprises the amino acid sequence of SEQ ID NO: 151 and the VL region of the antibody comprises the amino acid sequence of SEQ ID NO: 152; or
the VH region of the at least one antibody comprises the amino acid sequence of SEQ ID NO: 153 and the VL region of the antibody comprises the amino acid sequence of SEQ ID NO: 154.

In a further preferred embodiment the at least one primary antibody specific for a flavivirus envelope protein (E protein) antigen is an anti-DENV4 envelope protein antibody, preferably the at least one antibody is an antibody according to the present invention. In another preferred embodiment the at least one primary antibody present in a first well of the assay plate comprises a mixture of
(i) a primary antibody specific for a virus antigen from a first dengue virus serotype, wherein the primary antibody is provided at a first site of the well, and
(ii) a primary antibody specific for a virus antigen from a second dengue virus serotype, wherein the primary antibody is provided at a second site of the same well.

Preferably, the (i) secondary antibody specific for the E protein antigen is selected from clone DV-78 (2BScientific Catalog No: AB00246-10⁻⁰-BT).

In another preferred embodiment the (ii) secondary antibody specific for a portion of the pr domain of the prM protein may be selected from clone 2H2 (ATCC Catalog No: HB-114) and DV 62.5 (GenScript: U043WIB080-3). The 2H2 antibody is a pan-antibody binding to the pr domain of any of DENV1 to DENV4. The DV 62.5 antibody has been determined to bind to the pr domain of the dengue virus prM protein.

A key determinant of flaviviral infectivity is the proteolytic maturation of the virus-associated structural precursor membrane (prM) glycoprotein, which is cleaved by host protease(s) as nascent virions traffic through the secretory pathway. While this proteolysis is thought to be primarily mediated by the ubiquitous membrane-anchored cellular proprotein convertase (PC) furin, prM proteolysis mediated by other human PCs has not been conclusively ruled out. The currently accepted model for flaviviral prM activation proposes that prM endoproteolysis is mediated in the trans-Golgi network (TGN) by furin, yielding two products: soluble pr, and membrane-anchored M protein. Furin is predominantly localized to the TGN at steady state. However, furin is not statically retained in the TGN; it traffics between two local cycling loops, one at the TGN and the other at the cell surface. The prM proteolysis event is required for the fusogenicity of the virion, allowing pr to dissociate from its interaction with domain II of the flaviviral E protein and exposing the fusion peptide. The product of the prM proteolysis is therefore the mature virus.

For an improved characterization of the monovalent drug substance comprising a single live, attenuated dengue virus and the tetravalent drug product comprising a mixture of all four live, attenuated viruses allowing an improved consistency of the manufacturing process it is relevant to determine the maturation degree of the drug substance or the drug product. The average maturation degree may be considered to be the ratio of the proportion of mature virus in a sample as compared to the entirety of the viruses comprising mature and immature viruses contained in the sample.

### Anti-DENV2 Antibodies

In a further aspect the present invention provides an antibody specific for Dengue virus serotype 2 envelope protein (DENV2 E protein) or an antigen binding fragment thereof, wherein
(i) the VH CDR1 region of the antibody or antigen binding fragment thereof is selected from the group consisting of SEQ ID NO: 106, SEQ ID NO: 109, and SEQ ID NO: 112, or a variant thereof having at least 85% identity;
(ii) the VH CDR2 region of the antibody or binding fragment thereof is selected from the group consisting of SEQ ID NO: 107, SEQ ID NO: 110, and SEQ ID NO: 113, or a variant thereof having at least 85% identity;
(iii) the VH CDR3 region of the antibody or antigen binding fragment thereof is selected from the group consisting of SEQ ID NO: 108, SEQ ID NO: 111, and SEQ ID NO 114, or a variant thereof having at least 85% identity;
(iv) the VL CDR1 region of the antibody or antigen binding fragment thereof is selected from the group consisting of SEQ ID NO: 127, SEQ ID NO: 129, and SEQ ID NO: 131, or a variant thereof having at least 82% identity;
(v) the VL CDR2 region of the antibody or antigen binding fragment thereof is selected from the group of an amino acid sequence consisting of RAS, QAS and LAS, or a variant thereof having at least 65% identity; and
(vi) the VL CDR3 region of the antibody or antigen binding fragment thereof is selected from the group consisting of SEQ ID NO: 128, SEQ ID NO: 130, and SEQ ID NO: 132, or a variant thereof having at least 85% identity, wherein the antibody or antigen binding fragment thereon does not cross-react with other dengue serotypes other than DENV2 and has one or more of the following properties:
(1) a binding activity for DENV2-VLP calculated as EC₅₀ value of 38 ng/ml or less; and/or
(2) a k_{off} value of 1×10⁻⁴ sec⁻¹ or less.

“DENV2” includes any DENV serotype 2 virus strain. This serotype is segregated into seven subtypes. Preferably, the dengue virus serotype 2 strain is Thailand/16681/84 (Accession No. AAP06254.1).

“an antibody”: As is known in the art, an “antibody” is an immunoglobulin that binds specifically to a particular antigen. The term encompasses immunoglobulins that are naturally produced in that they are generated by an organism reacting to the antigen, and also those that are synthetically produced or engineered. An antibody may be monoclonal or polyclonal. An antibody may be a member of any immunoglobulin class, including any of the human classes: IgG, IgM, IgA, and IgD. A typical immunoglobulin (antibody) structural unit as understood in the art, is known to comprise a tetramer. Each tetramer is composed of two identical pairs of polypeptide chains, each pair having one “light” (approximately 25 kD) and one “heavy” chain (approximately 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains respectively. Each variable region is further subdivided into hypervariable (HV) and framework (FR) regions. The hypervariable regions comprise three areas of hypervariability sequence called complementarity determining regions (CDR 1, CDR 2 and CDR 3), separated by four framework regions (FR1, FR2, FR2, and FR4) which form a beta-sheet structure and serve as a scaffold to hold the HV regions in position. The C-terminus of each heavy and light chain defines a constant region consisting of one domain for the light chain (CL) and three for the heavy chain (CH1, CH2 and CH3). In some embodiments, the term “full length” is used in reference to an antibody to mean that it contains two heavy chains and two light chains, optionally associated by disulfide bonds as occurs with naturally-produced antibodies. In some embodiments, an antibody is produced by a cell. In some embodiments, an antibody is produced by chemical synthesis. In some embodiments, an antibody is derived from a mammal. In some embodiments, an antibody is derived from an animal such as, but not limited to, mouse, rat, horse, pig, or goat. In some embodiments, an antibody is produced using a recombinant cell culture system. In some embodiments, an antibody may be a purified antibody (for example, by immune-affinity chromatography). In some embodiments, an antibody may be a human antibody. In some embodiments, an antibody may be a humanized antibody (antibody from non-human species whose protein sequences have been modified to increase their similarity to antibody variants produced naturally in humans). In some embodiments, an antibody may be a chimeric antibody (antibody made by combining genetic material from a non-human source, e.g., mouse, rat, horse, or pig, with genetic material from humans).

“specific for DENV2” herein means that the antibody or antigen binding fragment significantly binds to an antigen of dengue virus serotype 2 as compared to a non-specific background. The skilled person is aware of several techniques for testing specific binding of an antibody. The antigen to which the antibody binds may be a structural or non-structural protein of dengue virus. Preferably, the antigen is the envelope protein of dengue virus. The envelope protein is characterized by three structural domains, EI, EII and EIII. More preferably, the antibody or antigen binding fragment thereof binds specifically to domain III of the dengue envelope protein.

“an antigen binding fragment thereof”: As used herein, an “antibody fragment” includes a portion of an intact antibody, such as, for example, the antigen-binding or variable region of an antibody. Examples of antibody fragments include Fab, Fab′, F(ab′)2, and Fv fragments; triabodies; tetrabodies; linear antibodies; single-chain antibody molecules; and multi specific antibodies formed from antibody fragments. For example, antibody fragments include isolated fragments, “Fv” fragments, consisting of the variable regions of the heavy and light chains, recombinant single chain polypeptide molecules in which light and heavy chain variable regions are connected by a peptide linker (“ScFv proteins”), and minimal recognition units consisting of the amino acid residues that mimic the hypervariable region. In many embodiments, an antibody fragment contains sufficient sequence of the parent antibody of which it is a fragment that it binds to the same antigen as does the parent antibody; in some embodiments, a fragment binds to the antigen with a comparable affinity to that of the parent antibody and/or competes with the parent antibody for binding to the antigen. Examples of antigen binding fragments of an antibody include, but are not limited to, Fab fragment, Fab′ fragment. F(ab′)2 fragment, scFv fragment, Fv fragment, dsFv diabody, dAb fragment, Fd′ fragment. Fd fragment, and an isolated complementarity determining region (CDR) region. An antigen binding fragment of an antibody may be produced by any means. For example, an antigen binding fragment of an antibody may be enzymatically or chemically produced by fragmentation of an intact antibody and/or it may be recombinantly produced from a gene encoding the partial antibody sequence. Alternatively or additionally, antigen binding fragment of an antibody may be wholly or partially synthetically produced. An antigen binding fragment of an antibody may optionally comprise a single chain antibody fragment.

Alternatively or additionally, an antigen binding fragment of an antibody may comprise multiple chains which are linked together, for example, by disulfide linkages. An antigen binding fragment of an antibody may optionally comprise a multimolecular complex. A functional antibody fragment typically comprises at least about 50 amino acids and more typically comprises at least about 200 amino acids. Preferably, the antigen binding fragment thereof can be obtained by screening of fragments for specific binding to the dengue envelope protein, more preferably for the domain III of the dengue envelope protein.

The variant of the indicated CDR sequences has an identity of at least 85%, preferably of at least 90% and even more preferred of at least 95%, and most preferred of at least 98% compared to the indicated CDR sequences. A variant includes one or more amino acid substitution, insertion and/or deletion compared to the indicated CDR sequences.

In a preferred embodiment the antibody or antigen binding fragment thereof has a binding activity for DENV2-VLP calculated as EC₅₀ value of 20 ng/ml or less.

“binding activity for DENV2-VLP”: DENV2-VLP has been selected as a model for intact dengue virus serotype 2 particles; see also Metz et al., Virol. J. 15 (2018), 60. It is considered that the three-dimensional form of the proteins on the surface of the VLP is in the native form correspond to the three-dimensional form of the proteins on the surface of the live virus. The skilled person knows how to produce dengue virus VLPs.

In another preferred embodiment the binding activity for DENV2-VLP is determined in an immunometric assay such as an ELISA, fluorescence or chemiluminescence assay, preferably DENV-2 VLP is derived from DENV2 strain Thailand/16681/84 (Accession No. AAP06254.1), more preferably the assay is in Luminex assay format.

DENV2 VLPs are also commercially available from e.g. the company Native Antigen. The VLP may be attached to a surface of a plate for carrying out the assay. Suitable assay forms include RIA, ELISA, or a chemiluminescent assay. Preferably, the binding of the antibody or antigen binding fragment thereof is determined in a Luminex assay format (Nascimento et al. Development and Characterization of a Multiplex Assay to Quantify Complement-Fixing Antibodies against Dengue Virus. Int J Mol Sci 22 (2021), 12004). One suitable format is described in the examples. The binding activity is calculated as EC₅₀ value. The EC₅₀ value corresponds to the antibody concentration, wherein 50% of the maximal binding of the antibody is observed.

In a further preferred embodiment the antibodies according to the present invention have a binding activity calculated as EC₅₀ value of 50 ng/ml or less, and even more preferred of 30 ng/ml or less.

In another preferred embodiment the antibody or antigen binding fragment thereof has a k_{off} value of 5×10⁻⁵ sec⁻¹ or less, preferably of 2×10⁻⁵ sec⁻¹ or less.

Binding kinetics relates to the rate at which the binding sites at a molecule such as an antibody are occupied with the ligand molecules such as antigens, i.e. the formation of the binding complex (association rate kₒₙ) and to the rate at which the ligand molecules are released from the binding sites, i.e. the dissociation of the binding complex (dissociation rate k_{off}). In the following k_{off} is also termed k_{off} value.

According to a preferred embodiment the association rate kₒₙ is measured in a biosensor format such as surface plasmon resonance (SPR) or biolayer interferometry (BLI). Preferably, BLI is used. The association rate is measured when the binding sites attached to the biosensor are contacted with a solution containing the ligand molecules. According to a preferred embodiment the dissociation rate k_{off} is measured when the biosensor with the binding complex is removed from the above solution and introduced into a solution which does not contain the ligand molecules such as a buffer solution. Methods for measuring the k_{off} value are described e.g. in WO 2021/067714 A2. Preferably, the k_{off} value is determined as described in the present examples.

In a preferred embodiment the dissociation rate k_{off} value is determined by biolayer interferometry (BLI), preferably the dissociation rate of the antibody or antigen binding fragment thereof is determined with respect to DENV2-VLP.

In a further preferred embodiment the binding activity of the antibody or antibody fragment is increased as compared to the clone DV10 (Absolute Antibody, Catalog No: Ab00245-23.0).

It is also preferred that the antibody of antigen binding fragment thereof does not cross-react with Zika virus.

“the antibody is not cross-reactive with Zika virus” herein means that in a preferred embodiment the antibody or antibody fragment thereof according to the invention does essentially not bind to Zika virus. The skilled person is aware of methods for testing the binding of antibodies to antigens. Suitable assays include, but are not limited to ELISA, RIA, luminex assay and avidity assay. As antigen, the Zika virus may be bound to the plate surface. Alternatively, Zika virus VLP (virus-like particle) may be used. The prior art anti-DENV2 antibodies are characterized by cross-reactivity to Zika virus which impairs for example diagnostic applications to distinguish superinfections by a dengue virus and a Zika virus from a single dengue infection. This drawback of the prior art is overcome by this preferred embodiment. A further application may be as control in the development of dengue specific vaccines.

Particularly preferred are the following combinations of VH CDR1 to CDR3 and VL CDR1 to CDR3. These combinations are based on the antibody clones identified in the examples as 3H11, 5F4 and 8D8, respectively.

TABLE 1
VH CDR1 to CDR3 Sequences

| Clone | CDR1 | CDR2 | CDR3 |
|---|---|---|---|
| 3H11 | GFSLINYD | VSGGGHT | GRWDI |
| | (SEQ ID | (SEQ ID | (SEQ ID |
| | NO: 106) | NO: 107) | NO: 108) |
| 5F4 | GIDLSNTV | ITPSGSA | ARIAWSTGRNDI |
| | (SEQ ID | (SEQ ID | (SEQ ID |
| | NO: 109) | NO: 110) | NO: 111) |
| 8D8 | GIDLSSTI | IGSSGSI | ARIAWSTGRNDI |
| | (SEQ ID | (SEQ ID | (SEQ ID |
| | NO: 112) | NO: 113) | NO: 114) |

TABLE 2
VH FWR1 to FWR4 Sequences

| Clone | FWR1 | FWR2 | FWR3 | FWR4 |
|---|---|---|---|---|
| 3H11 | QSVEESGGRLVT | MSWIRQAP | NYASWANGRFTISKTSSTT | WGPGT |
| | PGTPLTLTCTVS | GKGLEWIG | MDLKITSPTTEDTASYFC | LVTVSL |
| | (SEQ ID NO: | M (SEQ ID | (SEQ ID NO: 117) | (SEQ ID |
| | 115) | NO: 116) | | NO: 118) |
| 5F4 | QSVEESGGRLVK | ISWVRQAPG | YSATWAKGRVTISRTSTTV | WGPGTLVT |
| | PDETLTLTCTVS | KGLEYIGM | DLKIASPTTEDTATYFC | VSS |
| | (SEQ ID NO: | (SEQ ID NO: | (SEQ ID NO: 121) | (SEQ ID |
| | 119) | 120) | | NO: 122) |
| 8D8 | QSLEESGGRLVK | ITWVRQAPG | ISANWAKGRFTISRTSTTV | WGPGTL |
| | PDETLTLTCTVS | KGLEYIGI | DLKMTSLTTEDTATYFC | VTVSS |
| | (SEQ ID NO: | (SEQ ID NO: | (SEQ ID NO: 125) | (SEQ ID |
| | 123) | 124) | | NO: 126) |

TABLE 3
VL CDR1 to CDR3 sequences

| Clone | CDR1 | CDR2 | CDR3 |
|---|---|---|---|
| 3H11 | QSVYDDNW | RAS | LGGYVDDGDTT |
| | (SEQ ID | | (SEQ ID NO: 128) |
| | NO: 127) | | |
| 5F4 | QSVYNNNA | QAS | LGIYDDDVDNT |
| | (SEQ ID | | (SEQ ID NO: 130) |
| | NO: 129) | | |
| 8D8 | QSVYNNNA | LAS | LGVDDDDVDNT |
| | (SEQ ID | | (SEQ ID NO: 132) |
| | NO: 131) | | |

TABLE 4
VL FWR1 to FWR4 sequences

| Clone | FWR1 | FWR2 | FWR3 | FWR4 |
|---|---|---|---|---|
| 3H11 | AVVVTQTASPVSAAVG | LAWYQQKPGQ | RLASGVPSRFSG | FGGGTEVVVK |
| | GTVTISCQSS (SEQ ID | PPKQLIY (SEQ | TGSGTQFTLTISG | (SEQ ID NO: |
| | NO: 133) | ID NO: 134) | VQCDDAATYYC | 136) |
| | | | (SEQ ID NO: | |
| | | | 135) | |
| 5F4 | AAVLTQTPPSVSAAVGG | LSWYQQKPGQP | KLSSGVSSRFSGS | FGGGTEVVVG |
| | TVTIKCQSS (SEQ ID | PKLLIY | GSGTHFTLTISGV | (SEQ ID NO: |
| | NO: 137) | (SEQ ID | QCDDAATYYC | 140) |
| | | NO: 138) | (SEQ ID NO: | |
| | | | 139) | |
| 8D8 | AAVLTQTPPSVSAAVGG | LSWYQQKPGQP | NLASGVPSRFSG | FGGGTEVVVK |
| | TVTISCQSS | PKLLIY | SGSGTQFTLTISG | (SEQ ID NO: |
| | (SEQ ID NO: 141) | (SEQ ID NO: | VQCDDAATYHC | 144) |
| | | 142) | (SEQ ID NO: | |
| | | | 143) | |

TABLE 5
VH amino acid sequences of particular clones

| Clone | |
|---|---|
| 3H11 | QSVEESGGRLVTPGTPLTLTCTVSG |
| | FSLINYDMSWIRQAPGKGLEWIGMV |
| | SGGGHTNYASWANGRFTISKTSSTT |
| | MDLKITSPTTEDTASYFCGRWDIWG |
| | PGTLVTVSL |
| | (SEQ ID NO: 145) |
| 5F4 | QSVEESGGRLVKPDETLTLTCTVSG |
| | IDLSNTVISWVRQAPGKGLEYIGMI |
| | TPSGSAYSATWAKGRVTISRTSTTV |
| | DLKIASPTTEDTATYFCARIAWSTG |
| | RNDIWGPGTLVTVSS |
| | (SEQ ID NO: 146) |
| 8D8 | QSLEESGGRLVKPDETLTLTCTVSG |
| | IDLSSTIITWVRQAPGKGLEYIGII |
| | GSSGSIISANWAKGRFTISRTSTTV |
| | DLKMTSLTTEDTATYFCARIAWSTG |
| | RNDIWGPGTLVTVSS |
| | (SEQ ID NO: 147) |

TABLE 6
VL amino acid sequences of particular clones

| Clone | |
|---|---|
| 3H11 | AVVVTQTASPVSAAVGGTVTISCQS |
| | SQSVYDDNWLAWYQQKPGQPPKQLI |
| | YRASRLASGVPSRFSGTGSGTQFTL |
| | TISGVQCDDAATYYCLGGYVDDGDT |
| | TFGGGTEVVVK |
| | (SEQ ID NO: 148) |
| 5F4 | AAVLTQTPPSVSAAVGGTVTIKCQS |
| | SQSVYNNNALSWYQQKPGQPPKLLI |
| | YQASKLSSGVSSRFSGSGSGTHFTL |
| | TISGVQCDDAATYYCLGIYDDDVDN |
| | TFGGGTEVVVG |
| | (SEQ ID NO: 149) |
| 8D8 | AAVLTQTPPSVSAAVGGTVTISCQS |
| | SQSVYNNNALSWYQQKPGQPPKLLI |
| | YLASNLASGVPSRFSGSGSGTQFTL |
| | TISGVQCDDAATYHCLGVDDDDVDN |
| | TFGGGTEVVVK |
| | (SEQ ID NO: 150) |

In a preferred embodiment the antibody or antigen binding fragment thereof comprises the heavy chain CDR regions 1 to 3 having the amino acid sequences of SEQ ID Nos: 106 to 108, respectively, the light chain CDR region 1 has the amino acid sequence of SEQ ID NO: 127, the light chain CDR region 2 has the amino acid sequence RAS, and the light chain CDR region 3 has the amino acid sequence SEQ ID NO: 128.

In a further preferred embodiment the antibody of any one of claims 30 to 34, Wherein the antibody or antigen binding fragment thereof comprises the heavy chain CDR regions 1 to 3 having the amino acid sequences of SEQ ID Nos: 109 to 111, respectively, the light chain CDR region 1 has the amino acid sequence of SEQ ID NO: 129, the light chain CDR region 2 has the amino acid sequence QAS, and the light chain CDR region 3 has the amino acid sequence SEQ ID NO: 130.

In a further preferred embodiment the antibody of any one of claims 30 to 34, wherein the antibody or antigen binding fragment thereof comprises the heavy chain CDR regions 1 to 3 having the amino acid sequences of SEQ ID Nos: 112 to 114, respectively, the light chain CDR region 1 has the amino acid sequence of SEQ ID NO: 131, the light chain CDR region 2 has the amino acid sequence LAS, and the light chain CDR region 3 has the amino acid sequence SEQ ID NO: 132.

In a further preferred embodiment the antibody or antigen binding fragment thereof comprises in the VH domain the amino acid sequence set forth in SEQ ID NO: 145 or a derivative thereof, and in the VL domain the amino acid sequence set forth in SEQ ID NO: 148 or a derivative thereof, wherein the derivative differs from the indicated sequence by not more than five amino acid mutations.

In a further preferred embodiment the antibody or antigen binding fragment thereof comprises in the VH domain the amino acid sequence set forth in SEQ ID NO: 146 or a derivative thereof, and in the VL domain the amino acid sequence set forth in SEQ ID NO: 149 or a derivative thereof, wherein the derivative differs from the indicated sequence by not more than five amino acid mutations.

In a further preferred embodiment the antibody or antigen binding fragment thereof comprises in the VH domain the amino acid sequence set forth in SEQ ID NO: 147 or a derivative thereof, and in the VL domain the amino acid sequence set forth in SEQ ID NO: 150 or a derivative thereof, wherein the derivative differs from the indicated sequence by not more than five amino acid mutations.

Even more preferred, the derivative differs by not more than three amino acid mutations from the indicated sequence, in particular not more than one amino acid mutation from the indicated sequence. The amino acid mutations may be selected from one or more additions, deletions and/or substitutions.

### Anti-DENV4 Antibodies

In a further aspect the present invention provides an antibody specific for Dengue virus serotype 4 envelope protein (DENV4 E protein) or an antigen binding fragment thereof, wherein
(i) the VH CDR1 region of the antibody or antigen binding fragment thereof is selected from the group consisting of SEQ ID NO: 1, SEQ ID NO: 4, SEQ ID NO: 7, SEQ ID NO: 10, SEQ ID NO: 13, SEQ ID NO: 16 and SEQ ID NO: 19, or a variant thereof having at least 85% identity;
(ii) the VH CDR2 region of the antibody or binding fragment thereof is selected from the group consisting of SEQ ID NO: 2, SEQ ID NO: 5, SEQ ID NO: 8, SEQ ID NO: 11, SEQ ID NO: 14, SEQ ID NO: 17 and SEQ ID NO: 20, or a variant thereof having at least 85% identity;
(iii) the VH CDR3 region of the antibody or antigen binding fragment thereof is selected from the group consisting of SEQ ID NO: 3, SEQ ID NO: 6, SEQ ID NO: 9, SEQ ID NO: 12, SEQ ID NO: 15, SEQ ID NO: 18 and SEQ ID NO: 21, or a variant thereof having at least 85% identity;
(iv) the VL CDR1 region of the antibody or antigen binding fragment thereof is selected from the group consisting of SEQ ID NO: 50, SEQ ID NO: 52, SEQ ID NO: 54 and SEQ ID NO: 56, SEQ ID NO: 58, SEQ ID NO: 60 and SEQ ID NO: 62 or a variant thereof having at least 82% identity;
(v) the VL CDR2 region of the antibody or antigen binding fragment thereof is selected from the group of an amino acid sequence consisting of RAS, EAS and RAF, or a variant thereof having at least 65% identity; and
(vi) the VL CDR3 region of the antibody or antigen binding fragment thereof is selected from the group consisting of SEQ ID NO: 51, SEQ ID NO: 53, SEQ ID NO: 55 and SEQ ID NO: 57, SEQ ID NO: 59, SEQ ID NO: 61 and SEQ ID NO: 63, or a variant thereof having at least 85% identity,
wherein the antibody or antigen binding fragment thereon does not cross-react with other dengue serotypes other than DENV2 and has one or more of the following properties:
(1) a binding activity for DENV4-VLP calculated as EC₅₀ value of 80 ng/ml or less; and/or
(2) a k_{off} value of 1×10⁻⁴ sec⁻¹ or less.

“DENV4” includes any DENV serotype 4 virus strain. This serotype is segregated into subtypes I to IV. A detailed review can be found in Messer et al., Emerg. Infect. Diseases Vol. 9 (2003), pages 800-809. Preferably, the dengue virus strain is Dominica/814669/1981 (Accession No.: P09866.2).

“specific for DENV4” herein means that the antibody or antigen binding fragment significantly binds to an antigen of dengue virus serotype 3 as compared to a non-specific background. The skilled person is aware of several techniques for testing specific binding of an antibody. The antigen to which the antibody binds may be a structural or non-structural protein of dengue virus. Preferably, the antigen is the envelope protein of dengue virus. The envelope protein is characterized by three structural domains, EI, ELI and EIII. More preferably, the antibody or antigen binding fragment thereof binds specifically to domain III of the dengue envelope protein.

In a preferred embodiment the antibody or antigen binding fragment thereof has a binding activity for DENV4-VLP calculated as EC₅₀ value of 45 ng/ml or less and more preferably 30 ng/ml or less.

“binding activity for DENV4-VLP”: DENV4-VLP has been selected as a model for intact dengue virus serotype 4 particles; see also Metz et al., Virol. J. 15 (2018), 60. It is considered that the three-dimensional form of the proteins on the surface of the VLP is in the native form correspond to the three-dimensional form of the proteins on the surface of the live virus. The skilled person knows how to produce dengue virus VLPs.

In a preferred embodiment the binding activity for DENV4-VLP is determined in an immunometric assay such as an ELISA, fluorescence or chemiluminescence assay, preferably DENV-4 VLP is derived from DENV4 strain Dominica/814669/1981 (Accession No.: P09866.2), more preferably the assay is in Luminex assay format.

DENV4 VLPs are also commercially available from e.g. the company Native Antigen. The VLP may be attached to a surface of a plate for carrying out the assay. Suitable assay forms include RIA, ELISA, or a chemiluminescent assay. Preferably, the binding of the antibody or antigen binding fragment thereof is determined in a Luminex assay format (Nascimento et al. Development and Characterization of a Multiplex Assay to Quantify Complement-Fixing Antibodies against Dengue Virus. Int J Mol Sci 22 (2021), 12004). One suitable format is described in the examples. The binding activity is calculated as EC₅₀ value. The EC₅₀ value corresponds to the antibody concentration, wherein 50% of the maximal binding of the antibody is observed.

In a preferred embodiment the antibody or antigen binding fragment thereof has a k_{off} value of 5×10⁻⁵ sec⁻¹ or less, preferably of 2×10⁻⁵ sec⁻¹ or less.

According to a preferred embodiment the association rate kₒₙ is measured in a biosensor format such as surface plasmon resonance (SPR) or biolayer interferometry (BLI). Preferably, BLI is used. The association rate is measured when the binding sites attached to the biosensor are contacted with a solution containing the ligand molecules. According to a preferred embodiment the dissociation rate k_{off} is measured when the biosensor with the binding complex is removed from the above solution and introduced into a solution which does not contain the ligand molecules such as a buffer solution. Methods for measuring the k_{off} value are described e.g. in WO 2021/067714 A2. Preferably, the k_{off} value is determined as described in the present examples.

In a preferred embodiment the dissociation rate k_{off} value is determined by biolayer interferometry (BLI), preferably the dissociation rate of the antibody or antigen binding fragment thereof is determined with respect to DENV4-VLP.

In a further preferred embodiment the antibody or antigen binding fragment thereof has a binding activity for DENV4-VLP calculated as EC₅₀ value of 50 ng/ml or less and a k_{off} value of less than 5×10⁻⁵ sec⁻¹.

In a preferred embodiment the binding activity of the antibody or antibody fragment is increased as compared to the clone DV22 (source company: Absolute Antibody).

In a further preferred embodiment the antibody or antigen binding fragment thereof does not cross-react with Zika virus.

In a particularly preferred embodiment the antibody or antigen binding fragment thereof comprises the heavy chain CDR regions 1 to 3 having the amino acid sequences of SEQ ID Nos: 1 to 3, respectively, the light chain CDR region 1 has the amino acid sequence of SEQ ID NO: 50, the light chain CDR region 2 has the amino acid sequence RAS, and the light chain CDR region 3 has the amino acid sequence SEQ ID NO: 51.

In a particularly preferred embodiment the antibody or antigen binding fragment thereof comprises the heavy chain CDR regions 1 to 3 having the amino acid sequences of SEQ ID Nos: 4 to 6, respectively, the light chain CDR region 1 has the amino acid sequence of SEQ ID NO: 52, the light chain CDR region 2 has the amino acid sequence EAS, and the light chain CDR region 3 has the amino acid sequence SEQ ID NO: 53.

In a particularly preferred embodiment the antibody or antigen binding fragment thereof comprises the heavy chain CDR regions 1 to 3 having the amino acid sequences of SEQ ID Nos: 7 to 9, respectively, the light chain CDR region 1 has the amino acid sequence of SEQ ID NO: 54, the light chain CDR region 2 has the amino acid sequence RAS, and the light chain CDR region 3 has the amino acid sequence SEQ ID NO: 55.

In a particularly preferred embodiment the antibody or antigen binding fragment thereof comprises the heavy chain CDR regions 1 to 3 having the amino acid sequences of SEQ ID Nos: 10 to 12, respectively, the light chain CDR region 1 has the amino acid sequence of SEQ ID NO: 56, the light chain CDR region 2 has the amino acid sequence RAF, and the light chain CDR region 3 has the amino acid sequence SEQ ID NO: 57.

In a particularly preferred embodiment the antibody or antigen binding fragment thereof comprises the heavy chain CDR regions 1 to 3 having the amino acid sequences of SEQ ID Nos: 13 to 15, respectively, the light chain CDR region 1 has the amino acid sequence of SEQ ID NO: 58, the light chain CDR region 2 has the amino acid sequence EAS, and the light chain CDR region 3 has the amino acid sequence SEQ ID NO: 59.

In a particularly preferred embodiment the antibody or antigen binding fragment thereof comprises the heavy chain CDR regions 1 to 3 having the amino acid sequences of SEQ ID Nos: 16 to 18, respectively, the light chain CDR region 1 has the amino acid sequence of SEQ ID NO: 60, the light chain CDR region 2 has the amino acid sequence RAS, and the light chain CDR region 3 has the amino acid sequence SEQ ID NO: 61.

In a particularly preferred embodiment the antibody or antigen binding fragment thereof comprises the heavy chain CDR regions 1 to 3 having the amino acid sequences of SEQ ID Nos: 19 to 21, respectively, the light chain CDR region 1 has the amino acid sequence of SEQ ID NO: 62, the light chain CDR region 2 has the amino acid sequence RAS, and the light chain CDR region 3 has the amino acid sequence SEQ ID NO: 63.

In a particularly preferred embodiment the antibody or antigen binding fragment thereof comprises in the VH domain the amino acid sequence set forth in SEQ ID NO: 92 or a derivative thereof, and in the VL domain the amino acid sequence set forth in SEQ ID NO: 99 or a derivative thereof, wherein the derivative differs from the indicated sequence by not more than five amino acid mutations.

In a particularly preferred embodiment the antibody or antigen binding fragment thereof comprises in the VH domain the amino acid sequence set forth in SEQ ID NO: 93 or a derivative thereof, and in the VL domain the amino acid sequence set forth in SEQ ID NO: 100 or a derivative thereof, wherein the derivative differs from the indicated sequence by not more than five amino acid mutations.

In a particularly preferred embodiment the antibody or antigen binding fragment thereof comprises in the VH domain the amino acid sequence set forth in SEQ ID NO: 94 or a derivative thereof, and in the VL domain the amino acid sequence set forth in SEQ ID NO: 101 or a derivative thereof, wherein the derivative differs from the indicated sequence by not more than five amino acid mutations.

In a particularly preferred embodiment the antibody or antigen binding fragment thereof comprises in the VH domain the amino acid sequence set forth in SEQ ID NO: 95 or a derivative thereof, and in the VL domain the amino acid sequence set forth in SEQ ID NO: 102 or a derivative thereof, wherein the derivative differs from the indicated sequence by not more than five amino acid mutations.

In a particularly preferred embodiment the antibody or antigen binding fragment thereof comprises in the VH domain the amino acid sequence set forth in SEQ ID NO: 96 or a derivative thereof, and in the VL domain the amino acid sequence set forth in SEQ ID NO: 103 or a derivative thereof, wherein the derivative differs from the indicated sequence by not more than five amino acid mutations.

In a particularly preferred embodiment the antibody or antigen binding fragment thereof comprises in the VH domain the amino acid sequence set forth in SEQ ID NO: 97 or a derivative thereof, and in the VL domain the amino acid sequence set forth in SEQ ID NO: 104 or a derivative thereof, wherein the derivative differs from the indicated sequence by not more than five amino acid mutations.

In a particularly preferred embodiment the antibody or antigen binding fragment thereof comprises in the VH domain the amino acid sequence set forth in SEQ ID NO: 98 or a derivative thereof, and in the VL domain the amino acid sequence set forth in SEQ ID NO: 105 or a derivative thereof, wherein the derivative differs from the indicated sequence by not more than five amino acid mutations.

In a particularly preferred embodiment the binding activity for DENV4-VLP is determined in an immunometric assay such as an ELISA, fluorescence or chemiluminescence assay, preferably DENV-4 VLP is derived from DENV4 strain Dominica/814669/1981 (Accession No.: P09866.2), more preferably the assay is in Luminex assay format.

TABLE 7
VH CDR1 to CDR3 Sequences

| Clone | CDR1 | CDR2 | CDR3 |
|---|---|---|---|
| Clone 5B11 | GFSLSDSA (SEQ ID | IYAGSGTT | ARATAYVGYGYGDDTFDP |
| | NO: 1) | (SEQ ID NO: | (SEQ ID NO: 3) |
| | | 2) | |
| Clone 7D5 | GIDLSSWE (SEQ ID | IFPSGTI | GRHGGSLYLNI |
| | NO: 4) | (SEQ ID | (SEQ ID NO: 6) |
| | | NO: 5) | |
| Clone 7F2 | GIDLSYYA (SEQ ID | IDTGDSA | GRSLSSYYDWDAFDP |
| | NO: 7) | (SEQ ID NO: | (SEQ ID NO: 9) |
| | | 8) | |
| Clone 7G4 | GFSLSSCA (SEQ ID | IYVDSDTT | ARTTAYVGGGYGDDTFDP |
| | NO: 10) | (SEQ ID NO: | (SEQ ID NO: 12) |
| | | 11) | |
| Clone 8C7 | GIDLSSYE (SEQ ID | IFPSGTI | GRHGGSLYLNI |
| | NO: 13) | (SEQ ID | (SEQ ID NO: 15) |
| | | NO: 14) | |
| Clone 9G8 | GFSFSYRYW (SEQ | IDTGNGDT | ARSDVTLTGYGYGNL (SEQ |
| | ID NO: 16) | (SEQ ID | ID NO: 18) |
| | | NO: 17) | |
| Clone 10A11 | GINLSFYA (SEQ ID | IDTDNSA | ARSLSSFYDWDAFDP (SEQ |
| | NO: 19) | (SEQ ID | ID NO: 21) |
| | | NO: 20) | |

TABLE 8
VH FWR1 to FWR4 Sequences

| Clone | FWR1 | FWR2 | FWR3 | FWR4 |
|---|---|---|---|---|
| DENV4_ | QSVEESGGRLVTP | MSWVRQAPG | YYASWAKGRFTISKTSTTVDLKI | WGPGTLVTVSS |
| 5B11 | GTPLTLTCTVS | KGLEWIGY | TSPTTEDTATYFC (SEQ ID NO: | (SEQ ID NO: 25) |
| | (SEQ ID NO: 22) | (SEQ ID NO: | 24) | |
| | | 23) | | |
| DENV4_ | QSVEESGGRLVTP | MGWVRQAPG | YYANWAKGRFAISKASTTVDL | WGPGTLVTVSL |
| 7D5 | GTPLTLTCTVS | KGLEWIGI | KITSPTTEDTATYFC (SEQ ID | (SEQ ID NO: 29) |
| | (SEQ ID NO: 26) | (SEQ ID NO: | NO: 28) | |
| | | 27) | | |
| DENV4_ | QSVEESGGRLVTP | MGWVRQAPG | YYASWAKGRFTISKTSSTTVDL | WGPGTLVTVSS |
| 7F2 | GTPLTLTCTVS | EGLECIGF (SEQ | KITSPTPEDTATYFC (SEQ ID | (SEQ ID NO: 33) |
| | (SEQ ID NO: 30) | ID NO: 31) | NO: 32) | |
| DENV4_ | QSVEESGGRLVTP | MTWVRQAPG | YYASWAKGRFTISTTSTTVDLRI | WGPGTLVTVSS |
| 7G4 | GTPLTLTCTVS | KGLEWIGY | TSPTTEDTATYFC (SEQ ID NO: | (SEQ ID NO: 37 ) |
| | (SEQ ID NO: 34) | (SEQ ID NO: | 36) | |
| | | 35) | | |
| DENV4_ | QSVEESGGRLVTP | MGWVRQAPG | YYANWAKGRFTISKTSTTVDLK | WGPGTLVTVSL |
| 8C7 | GTPLTLTCTVS | KGLEWIGI | ITSPTTEDTATYFC (SEQ ID | (SEQ ID NO: 41) |
| | (SEQ ID NO: 38) | (SEQ ID NO: | NO: 40) | |
| | | 39) | | |
| DENV4_ | QSLEESGGGLVKP | MSWVRQAPG | YYASRAKGRFTIMKTSSTTVTL | WGPGTLVTVSS |
| 9G8 | GASLTLTCTAS | KRPEWIAC | QMTSLTVADTATYFC (SEQ ID | (SEQ ID NO: 45) |
| | (SEQ ID NO: 42) | (SEQ ID NO: | NO: 44) | |
| | | 43) | | |
| DENV4_ | QSVEESGGRLVTP | MGWVRQAPG | YYASWAKGRFTISKTSTTVDLKI | WGPGTLVTVSS |
| 10A11 | GTPLTLTCTVS | KGLECVGF | TSPTTEDTATYFC (SEQ ID NO: | (SEQ ID NO: 49) |
| | (SEQ ID NO: 46) | (SEQ ID NO: | 48) | |
| | | 47) | | |

TABLE 9
VL CDR1 to CDR3 sequences

| clone | CDR1 | CDR2 | CDR3 |
|---|---|---|---|
| DENV4_ | QSISSY | RAS | QQGYSNSDVDNV |
| 5B11 | (SEQ ID | | (SEQ ID NO: 51) |
| | NO: 50) | | |
| DENV4_ | QSVYVNRN | EAS | QGEFGCSSADCIA |
| 7D5 | (SEQ ID | | (SEQ ID NO: 53) |
| | NO: 52) | | |
| DENV4_ | QTIGNL | RAS | QQGASANDIDNS |
| 7F2 | (SEQ | | (SEQ ID NO: 55) |
| | ID NO: 54) | | |
| DENV4_ | QNINSY (SEQ | RAF | QQGYSFSGVDNV |
| 7G4 | ID NO: 56) | | (SEQ ID NO: 57) |
| DENV4_ | QSVYVNRN | EAS | QGEFGCSSADCIA |
| 8C7 | (SEQ ID NO: | | (SEQ ID NO: 59) |
| | 58) | | |
| DENV4_ | HNIYN (SEQ | RAS | QQYYGGDDDGFG |
| 9G8 | ID NO: 60)S | | (SEQ ID NO: 61) |
| DENV4_ | QSISNL (SEQ | RAS | QQGASNSDIDNA |
| 10A11 | ID NO: 62) | | (SEQ ID NO: 63) |

TABLE 10
VL FWR1 to FWR4 sequences

| clone | FWR1 | FWR2 | FWR3 | FWR4 |
|---|---|---|---|---|
| DENV4_ | AYDMTQTPASVEVAV | LSWYQQKPG | TLASGVSSRFKGSGSGTEFTLT | FGGGTEVVVK |
| 5B11 | GGTVTIKCQAS (SEQ | QRPKLLIY | ISGVQCDDAATYYC (SEQ ID | (SEQ ID NO: 67) |
| | ID NO: 64) | (SEQ ID NO: | NO: 66) | |
| DENV4_ | AQVLTQTPSPVSAAV | LAWYQQKPG | KLASGVPSRFKGSGSGTQFTL | FGGGTEVVVK |
| 7D5 | GGTVTINCQAS (SEQ | QPPKVLIY | TISGVQCDDAATYYC (SEQ ID | (SEQ ID NO: 71) |
| | ID NO: 68) | (SEQ ID NO: | NO: 70) | |
| | | 69) | | |
| DENV4_ | AYDMTQTPASVEVAV | LAWYQQKPG | TLASGVPSRFKGSGSGTQFTL | FGGGTAVVVK |
| 7F2 | GGTVTINCQAS (SEQ | QRPKLLI (SEQ | TISGVECADAATYYC (SEQ ID | (SEQ ID NO: 75) |
| | ID NO: 72) | ID NO: 73) Y | NO: 74) | |
| DENV4_ | DVVMTQTPASVEAAV | LSWYQQKPG | TLASGVSSRFKGSGSGTQFTL | FGGGTEVVV K |
| 7G4 | GGTVTINCQAS (SEQ | QRPKLLIY | TISGVECADAATYYC (SEQ ID | (SEQ ID NO: 79) |
| | ID NO: 76) | (SEQ ID NO: | NO: 78) | |
| | | 77) | | |
| DENV4_ | AQVLTQTPSPVSAAV | LAWYQQKPG | KLASGVPSRFKGSGSGTQFTL | FGGGTEVVVK |
| 8C7 | GGTVTINCQAS (SEQ | QPPKVLIY | TISGVQCDDAATYYC (SEQ ID | (SEQ ID NO: 83) |
| | ID NO: 80) | (SEQ ID NO: | NO: 82) | |
| | | 81) | | |
| DENV4_ | DSVMTQTPASVSEPV | LAWYQQKPG | TLESGVPSRVKGSGYGTEYTLT | FGGGTEVVVK |
| 9G8 | GGTVTINCQAS (SEQ | QPPKLLIY | ISDLECADAATYYC (SEQ ID | (SEQ ID NO: 87) |
| | ID NO: 84) | (SEQ ID NO: | NO: 86) | |
| | | 85) | | |
| DENV4_ | AYDMTQTPASVEVAV | LAWYQQKPG | TLASGVPSRFKGSGSGTQFTL | FGGGTEVVVK |
| 10A11 | GGTVTIKCLAS (SEQ | QRPKLLIY | TISGVECADAATYYC (SEQ ID | (SEQ ID NO: 91) |
| | ID NO: 88) | (SEQ ID NO: | NO: 90) | |
| | | 89) | | |

TABLE 11
VH amino acid sequences of particular clones

| clone | VH (VDJ) |
|---|---|
| DENV4_ | QSVEESGGRLVTPGTPLTLTCTVSG |
| 5B11 | FSLSDSAMSWVRQAPGKGLEWIGYI |
| | YAGSGTTYYASWAKGRFTISKTSTT |
| | VDLKITSPTTEDTATYFCARATAYV |
| | GYGYGDDTFDPWGPGTLVTVSS |
| | (SEQ ID NO: 92) |
| DENV4_ | QSVEESGGRLVTPGTPLTLTCTVSG |
| 7D5 | IDLSSWEMGWVRQAPGKGLEWIGII |
| | FPSGTIYYANWAKGRFAISKASTTV |
| | DLKITSPTTEDTATYFCGRHGGSLY |
| | LNIWGPGTLVTVSL |
| | (SEQ ID NO: 93) |
| DENV4_ | QSVEESGGRLVTPGTPLTLTCTVSG |
| 7F2 | IDLSYYAMGWVRQAPGEGLECIGFI |
| | DTGDSAYYASWAKGRFTISKTSSTT |
| | VDLKITSPTPEDTATYFCGRSLSSY |
| | YDWDAFDPWGPGTLVTVSS |
| | (SEQ ID NO: 94) |
| DENV4_ | QSVEESGGRLVTPGTPLTLTCTVSG |
| 7G4 | FSLSSCAMTWVRQAPGKGLEWIGYI |
| | YVDSDTTYYASWAKGRFTISTTSTT |
| | VDLRITSPTTEDTATYFCARTTAYV |
| | GGGYGDDTFDPWGPGTLVTVSS |
| | (SEQ ID NO: 95) |
| DENV4_ | QSVEESGGRLVTPGTPLTLTCTVSG |
| 8C7 | IDLSSYEMGWVRQAPGKGLEWIGII |
| | FPSGTIYYANWAKGRFTISKTSTTV |
| | DLKITSPTTEDTATYFCGRHGGSLY |
| | LNIWGPGTLVTVSL |
| | (SEQ ID NO: 96) |
| DENV4_ | QSLEESGGGLVKPGASLTLTCTASG |
| 9G8 | FSFSYRYWMSWVRQAPGKRPEWIAC |
| | IDTGNGDTYYASRAKGRFTIMKTSS |
| | TTVTLQMTSLTVADTATYFCARSDV |
| | TLTGYGYGNLWGPGTLVTVSS |
| | (SEQ ID NO: 97) |
| DENV4_ | QSVEESGGRLVTPGTPLTLTCTVSG |
| 10A11 | INLSFYAMGWVRQAPGKGLECVGFI |
| | DTDNSAYYASWAKGRFTISKTSTTV |
| | DLKITSPTTEDTATYFCARSLSSFY |
| | DWDAFDPWGPGTLVTVSS |
| | (SEQ ID NO: 98) |

TABLE 12
VL amino acid sequences of particular clones

| clone | VL (VJ) |
|---|---|
| DENV4_ | AYDMTQTPASVEVAVGGTVTIKCQA |
| 5B11 | SQSISSYLSWYQQKPGQRPKLLIYR |
| | ASTLASGVSSRFKGSGSGTEFTLTI |
| | SGVQCDDAATYYCQQGYSNSDVDNV |
| | FGGGTEVVVK |
| | (SEQ ID NO: 99) |
| DENV4_ | AQVLTQTPSPVSAAVGGTVTINCQA |
| 7D5 | SQSVYVNRNLAWYQQKPGQPPKVLI |
| | YEASKLASGVPSRFKGSGSGTQFTL |
| | TISGVQCDDAATYYCQGEFGCSSAD |
| | CIAFGGGTEVVVK |
| | (SEQ ID NO: 100) |
| DENV4_ | AYDMTQTPASVEVAVGGTVTINCQA |
| 7F2 | SQTIGNLLAWYQQKPGQRPKLLIYR |
| | ASTLASGVPSRFKGSGSGTQFTLTI |
| | SGVECADAATYYCQQGASANDIDNS |
| | FGGGTAVVVK |
| | (SEQ ID NO: 101) |
| DENV4_ | DVVMTQTPASVEAAVGGTVTINCQA |
| 7G4 | SQNINSYLSWYQQKPGQRPKLLIYR |
| | AFTLASGVSSRFKGSGSGTQFTLTI |
| | SGVECADAATYYCQQGYSFSGVDNV |
| | FGGGTEVVVK |
| | (SEQ ID NO: 102) |
| DENV4_ | AQVLTQTPSPVSAAVGGTVTINCQA |
| 8C7 | SQSVYVNRNLAWYQQKPGQPPKVLI |
| | YEASKLASGVPSRFKGSGSGTQFTL |
| | TISGVQCDDAATYYCQGEFGCSSAD |
| | CIAFGGGTEVVVK |
| | (SEQ ID NO: 103) |
| DENV4_ | DSVMTQTPASVSEPVGGTVTINCQA |
| 9G8 | SHNIYNSLAWYQQKPGQPPKLLIYR |
| | ASTLESGVPSRVKGSGYGTEYTLTI |
| | SDLECADAATYYCQQYYGGDDDGFG |
| | FGGGTEVVVK |
| | (SEQ ID NO: 104) |
| DENV4_ | AYDMTQTPASVEVAVGGTVTIKCLA |
| 10A11 | SQSISNLLAWYQQKPGQRPKLLIYR |
| | ASTLASGVPSRFKGSGSGTQFTLTI |
| | SGVECADAATYYCQQGASNSDIDNA |
| | FGGGTEVVVK |
| | (SEQ ID NO: 105) |

In a further aspect a pair of nucleic acids comprising a first nucleic acid encoding the heavy chain of the antibody or antigen binding fragment and a second nucleic acid encoding the light chain of the antibody or antigen binding fragment is provided. Further, a vector comprising the first nucleic acid of and/or the second nucleic acid under the control of one or more suitable promoters is provided. The promoter may be a constitutive or inducible promoter. Suitable promoters are known to the skilled person.

In addition, a host cell transformed with at least one vector of the present invention and capable of expressing the antibody or antigen binding fragment is provided.

In a further aspect a method for the recombinant production of the antibody comprising culturing the transformed host cell under conditions suitable of expressing the antibody and optionally purifying the antibody from the culture medium is provided.

Methods for generating antibodies (e.g., monoclonal antibodies and/or polyclonal antibodies) are well known in the art. It will be appreciated that a wide range of animal species can be used for the production of antisera, including rabbit, mouse, rat, hamster, guinea pig or goat. The choice of animal may be decided upon the ease of manipulation, costs or the desired amount of sera, as would be known to one of skill in the art. It will be appreciated that the antibody or antigen binding fragment thereof can also be produced transgenically through the generation of a mammal or plant that is transgenic for the immunoglobulin heavy and light chain sequences of interest and production of the antibody in a recoverable form therefrom. In connection with the transgenic production in mammals, antibodies can be produced in, and recovered from, the milk of goats, cows, or other mammals. See, e.g., U.S. Pat. Nos. 5,827,690, 5,756,687, 5,750,172, and 5,741,957.

The antibody or antigen binding fragment thereof may be produced, for example, by utilizing a host cell system engineered to express an inventive antibody-encoding nucleic acid. Alternatively or additionally, provided antibody agents may be partially or fully prepared by chemical synthesis (e.g., using an automated peptide synthesizer).

Exemplary sources for the antibody or antigen binding fragment thereof suitable for the invention include, but are not limited to, conditioned culture medium derived from culturing a recombinant cell line that expresses a protein of interest, or from a cell extract of, e.g., antibody-producing cells, bacteria, fungal cells, insect cells, transgenic plants or plant cells, transgenic animals or animal cells, or serum of animals, ascites fluid, hybridoma or myeloma supernatants. Suitable bacterial cells include, but are not limited to, Escherichia coli cells. Examples of suitable E. coli strains include: HB101, DH5a, GM2929, JM109, KW251, NM538, NM539, and any E. coli strain that fails to cleave foreign DNA. Suitable fungal host cells that can be used include, but are not limited to, Saccharomyces cerevisiae, Pichia pastoris and Aspergillus cells. Suitable insect cells include, but are not limited to, S2 Schneider cells, D. Mel-2 cells, SF9, SF21, High-5™, Mimic™-SF9, MG1 and KC1 cells. Suitable exemplary recombinant cell lines include, but are not limited to, BALB/c mouse myeloma line, human retinoblasts (PER.C6), monkey kidney cells, human embryonic kidney line (293), baby hamster kidney cells (BHK), Chinese hamster ovary cells (CHO), mouse Sertoli cells, African green monkey kidney cells (VERO-76), human cervical carcinoma cells (HeLa), canine kidney cells, buffalo rat liver cells, human lung cells, human liver cells, mouse mammary tumor cells, TRI cells, MRC 5 cells, FS4 cells, and human hepatoma line (Hep G2).

The antibody or antigen binding fragment thereof can be expressed using various vectors (e.g., viral vectors) known in the art and cells can be cultured under various conditions known in the art (e.g., fed-batch). Various methods of genetically engineering cells to produce antibodies are well known in the art. See e.g. Ausabel et al, eds. (1990), Current Protocols in Molecular Biology (Wiley, New York).

The antibody or antigen binding fragment thereof may be purified, if desired, using filtration, centrifugation and/or various chromatographic methods such as HPLC or affinity chromatography. In some embodiments, fragments of the provided antibodies are obtained by methods which include digestion with enzymes, such as pepsin or papain, and/or by cleavage of disulfide bonds by chemical reduction.

The antibody or antigen binding fragment thereof may be coupled to a detectable marker.

In a further aspect an in vitro method for detecting DENV2 viruses or DENV4 viruses in a biological sample is provided, wherein the method comprises contacting the antibody with a biological sample and determining the amount of antibody bound to the biological sample. The in vitro method may be an enzymatic, fluorescent or chemiluminescent method. In a preferred embodiment, if the antibody or antibody fragment is not cross-reactive with Zika virus, it may be used as a diagnostic assay to distinguish a dengue monoinfection from a superinfection with dengue virus and Zika virus.

In a further aspect a kit for the detection of DENV2 or DENV4 viruses comprising an antibody or antigen binding fragment according to the present invention is provided. The kit may also contain instructions for carrying out the detection process.

In a further aspect a pharmaceutical formulation comprising the antibody or an antigen binding fragment thereof and optionally one or more pharmaceutically acceptable carrier is provided. As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate: agar; buffering agents, such as magnesium hydroxide and aluminum hydroxide; alginic acid: pyrogen-free water; isotonic saline; Ringer's solution: ethyl alcohol; pH buffered solutions; polyesters, polycarbonates and/or polyanhydrides; and other non-toxic compatible substances employed in pharmaceutical formulations.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue: parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally: or nasally, pulmonary, and to other mucosal surfaces.

In a further aspect an antibody or antigen binding fragment according to the present invention is provided for use in the prevention or treatment of a Dengue disease in a subject. The antibody or antigen binding fragment may be present in a therapeutically effective amount. As used herein, the term “therapeutically effective amount” refers to an amount of a therapeutic protein which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic protein or composition effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed: the age, body weight, general health, sex and diet of the patient; the time of administration, route of administration, and/or rate of excretion or metabolism of the specific fusion protein employed; the duration of the treatment; and like factors as is well known in the medical arts.

In a further aspect a method for the prevention or treatment of a Dengue disease in a subject is provided, wherein the method comprises administering the antibody or antigen binding fragment of the present invention. The above considerations for the use of the antibody or antigen binding fragment for the prevention or treatment of dengue disease apply equally. The subject is preferably a human.

The following examples are not intended to limit the present invention, but merely to illustrate particular preferred embodiments.

### EXAMPLES

### Materials and Methods

### Infectivity Assay

Vero cells in growth medium (1×DMEM phenol-free+10% FBS+25 mM HEPES, pH 7.2)) are added to each well of a 96-well plate. The plates are incubated for 18 to 24 hours at 37° C. Monovalent drug substance (each of TDV-1 to TDV-4, SEQ ID Nos: 1 to 4) separately) and tetravalent drug product (combination of TDV-1 to TDV-4) are diluted in infection medium (1×DMEM phenol-free+2% FBS+1% F127+25 mM HEPES, pH 7.2). The media are removed from the Vero cells. 30 μL of the prepared virus dilution series are added to each well of the 96 well plate. The plates are incubated for 4 hours at 37° C. to allow for infection. 100 μL of growth medium is added to all wells and incubated for 72 hours at 37° C. The media are removed from the infected cells. Lysis is performed by adding to 50 μL Tris Lysis Buffer (Meso Scale Discovery, Inc.) and incubated for 1 hour at 4° C.

The detection of the dengue envelope protein by immunoassay was performed as follows:

Assay plates (MSD Assay Plate; Meso Scale Discovery, Inc.) pre-coated with streptavidin were coated by adding 30 μL of biotinylated capture antibody. The capture antibody was a dengue serotype-specific antibody. The plates were incubated for 1 hour at room temperature on a plate shaker at 300 rpm. Thereafter, the plates were washed and 150 μL blocking buffer (MSD Blocker A; Meso Scale Discovery, Inc.) was added to each well and incubated for 1 hour at room temperature. The above described cell lysates were diluted fivefold in blocking buffer. Subsequently, 30 μL of the diluted cell lysates were transferred to the assay plate with the bound capture antibody and incubated for 2 hours at room temperature under shaking. After washing, 30 μL of detection antibody DV-78 (Absolute Antibody) diluted to 0.15 μg/mL in blocking buffer to the assay plate and incubate for 1 hour at room temperature on a plate shaker. Subsequently, 30 μL/well of secondary anti-rabbit sulfo-tag antibody (Meso Scale Discovery, Inc.) diluted 1000-fold in blocking buffer was added and incubated for 1 hour at room temperature.

To each well, 150 μL of 1× Read Buffer T (Meso Scale Discovery, Inc.) was added and reading was immediately conducted using an Imager Reader (S2400, S600 or equivalent; Meso Scale Discovery. Inc.). An EC₅₀ value was determined using JMP software.

### Content Assay

Serial dilutions of the monovalent attenuated dengue virus (TDV-1 to TDV-4) or the tetravalent combination of the attenuated dengue virus (each of TDV-1 to TDV-4) are prepared. The diluted samples were used in the same immunoassay method described above for the infectivity assay instead of the cell lysates.

### Results

### Infectivity Assay

The infectivity assay can be carried out such that the determination of the different dengue viruses is serotype-specific. This is demonstrated for TDV-1 to TDV-4 in FIGS. 1 to 4, respectively. Low cross-reactivity is observed with lysates from different serotypes indicating good specificity.

The results of the infectivity assay are strongly correlated with the results of the immunofocus assay which is the current gold standard for determining infectivity or potency of virus samples. The results are shown in FIG. 5.

### Content Assay

The proof of concept for determination of the dengue E protein content in virus samples is shown for the different TDV-1 to TDV-4, separately, is shown in FIGS. 6 to 9. The result for determination of the dengue E protein content in virus samples is shown for the combination comprising TDV-1, TDV-2, TDV-3 and TDV-4 is shown in FIG. 10.

The results of the content assay according to the present invention are strongly correlated with the results of the isotope dilution mass spectrometry analysis which has been used in the prior art for determining the total content of virus protein such as dengue envelope protein.

### Improvement of the Content Assay

### Methods

1 or 10 μg/mL Biotinylated 777-3, DV3-E60, DENV3-5D7, and DENV3-13E10 mAb were diluted in D100 Diluent (MSD), then 100 L/well of mAb solutions were incubated with MSD 96 well plates (Meso Scale Discovery) at room temperature for 60 min for 500 rpm shaking. Then, 150 μL/well of Blocking buffer (MSD) were added for each well and incubated at room temperature for 30 min for 500 rpm shaking. The blocking solution was applied and the plate was washed three times with PBS 0.05% Tween 20. Next 50 μL/well of various concentrations of Dengue 1, 2, 3 and 4 virus infected cell lysate with D100 diluent (MSD) was added to each well and incubated at room temperature for 120 min at shaking for 500 rpm. The cell lysate solution was added and the plate was washed three times with PBS 0.05% Tween 20. Then 100 μL/well of 4 μg ST (Sulfo-tag) labeled DV78/mL in D100 Diluent (MSD) were added to each well and incubated at room temperature for 60 min for 500 rpm shaking. The ST-DV78 solution was added and the plate was washed three times with PBS 0.05% Tween 20. 150 μL/well of Read buffer (MSD) for each well was added, and then the intensity was read using the MESO SECTOR S 600 MM (MSD).

### Results

When DENV3-5D7 was used for the captured antibody, a higher signal, around 700,000, was observed. However, 777-3 has low signals, around 200,000, and DV3-E60 did not show any signals (FIG. 2A). DENV3-5D7 showed Dengue 3 virus specific but not bound to other serotypes. Using the DENV3-5D7 mAb for the content assay may increase the sensitivity of the assays.

### Maturation Assay

### Methods

Assay plates (MSD Assay Plate; Meso Scale Discovery, Inc.) pre-coated with streptavidin were coated by adding 30 μL of biotinylated capture antibody. The capture antibody was a dengue serotype-specific antibody. The plates were incubated for 1 hour at room temperature on a plate shaker at 300 rpm. Thereafter, the plates were washed and 150 μL blocking buffer (MSD Blocker A; Meso Scale Discovery, Inc.) was added to each well and incubated for 1 hour at room temperature. Serial dilutions of the monovalent attenuated dengue virus (TDV-1 to TDV-4) were prepared. Subsequently, 30 μL of the diluted samples were transferred to the assay plate with the bound capture antibody and incubated for 2 hours at room temperature under shaking.

After washing, for the prM protein determination, 30 μL of detection antibody 2H2 (available e.g. from Sigma Aldrich) diluted to 0.15 μg/mL in blocking buffer to the assay plate and incubated for 1 hour at room temperature on a plate shaker. For the E protein determination, 30 μL of detection antibody DV78 diluted to 0.15 μg/mL in blocking buffer to the assay plate and incubated for 1 hour at room temperature on a plate shaker.

Subsequently, 30 μL/well of secondary anti-rabbit sulfo-tag antibody (Meso Scale Discovery, Inc.) diluted 1000-fold in blocking buffer was added and incubated for 1 hour at room temperature.

To each well, 150 μL of 1×Read Buffer T (Meso Scale Discovery, Inc.) was added and reading was immediately conducted using an Imager Reader (S2400, S600 or equivalent; Meso Scale Discovery. Inc.). An EC₅₀ value was determined using JMP software.

### Results

The results for the E protein and prM protein determination are shown in FIGS. 11 and 12, respectively. The prM/E ratio has been determined for each monovalent drug substance of TDV-1 to TDV-4.

TABLE 13

| Sample | prM/E ratio |
|---|---|
| DS TDV1 | 0.98 |
| DS TDV2 | 1.31 |
| DS TDV3 | 1.06 |
| DS TDV4 | 0.25 |

### Anti-DENV2 and Anti-DENV4 Antibodies

### Materials and Methods

### Antigens and Other Reagents

All VLP and proteins were purchased from The Native Antigen Company (Oxford, U.K.: Table 14).

TABLE 14
VLP and inactivated virus for immunized antigens

| Description | Source | Strain info | Accession No |
|---|---|---|---|
| DENV-2 VLP | Native Antigen | Thailand/16681/84 | AAP06254.1 |
| DENV-4 VLP | Native Antigen | Dominica/814669/1981 | P09866.2 |
| DENV2 inactivated virus | Microbix | Thailand/16681/84 | AAB58782.1 |

Inactivated dengue viruses were obtained from Microbix Biosystems (Mississauga, ON, Canada). RVP was purchased from Integral Molecular (Philadelphia PE, USA: Table 15).

TABLE 15
RVP reagents

| Description | Source | Strain info | Accession No |
|---|---|---|---|
| DENV-1 RVP | Intrgral Molecular | 16007/Thailand/1964 | QTX92473.1 |
| DENV-2 RVP | Intrgral Molecular | 16681/Thailand/1964 | NP_056776.2 |
| DENV-3 RVP | Intrgral Molecular | 16562/Philippines/1964 | U11673.1 |

Assay-Ready Raji DC-SIGN Cells (ARC) were purchased from Accellerate (Hamburg, Germany) [1] 5J7 and Mab513 was obtained from Creative Biolabs (Shirley, NY), DV10, DV18, DV63, DV78 and 4G2 were purchased from Absolute antibody (Oxford, UK) 777-3 (D6-8A1-12), 78-2 was expressed and purified from CHO cell, hybridoma cells and Expi293 cells, respectively (Table 16).

TABLE 16
Commercial and purified mAbs

| Clones | Binding epitopes | Manufacture |
|---|---|---|
| 2D73 | DIII | Absolute antibody |
| 3H5 | DIII | Absolute antibody |
| 5J7 | DII EDE | Creative biolab |
| Mab 513 | DIII | Creative biolab |
| D1-11(3) | DIII | ThermoFisher |
| DV10 | DIII | Absolute antibody |
| DV18 | DI/DII | Absolute antibody |
| DV22 | DI/II | Absolute antibody |
| DV63 | DIII | Absolute antibody |
| DV78 | DI/DII | Absolute antibody |
| 4G2 | DII Fusion Loop | Absolute antibody |
| 777-2 (DV2-3H5) | DIII | In house prep |
| 777-3 (D6-8A1-12) | Envelope protein | In house prep |
| 777-4 (1H10-6-7) | Envelope protein | In house prep |
| 78-2 | DII Fusion Loop | In house prep |

EDE: E dimer epitope
DI: Envelope protein Domain I,
DII: Domain II and
DIII: Domain III

### Animals and Immunizations

Two New Zealand White (NZW) female rabbits were immunized subcutaneously (S.Q.) with 200 μg DENV VLP with Freund's incomplete adjuvant or 200 μg DENV inactivated virus on day 0, 14, 28, and 42. The immunized rabbits collected the bleed on days 35 and 49 and confirmed the anti-sera titer against DENV VLP using Luminex assay or enzyme-linked immunosorbent assay (ELISA) neutralizing titer using RVP. One DENV2 immunized rabbit resumed immunization on day 120 and continued on day 140 with 200 μg DENV VLP with Freund's incomplete adjuvant. Selected rabbits were boosted with 400 μg DENV VLP or DENV inactivated virus by intravenous (IV) injection on day 59 or day 164. These immunized rabbits were sacrificed on day 64 or day 167 and collected spleen and bleeds. Spleen tissue was washed with an RPMI medium and dispersed to single cells by pipetting passed through a cell strainer. The single dispersed cell was conducted B cell sorting or stored in liquid nitrogen with a cell storage medium.

### Rabbit B Cell Sorting

For each sorting, ˜2×10⁸ freshly isolated splenocytes or 3 vials of thawed splenocytes (˜7×10⁷/vial) from selected rabbits were cultured in B-cell culture media (RPMI-1640, 15% FBS, 1×HEPES, 1×2-ME (1-Mercaptoethanol), 1% Penicillin/Streptomycin) overnight before sorting. 96-well B cell feeding plates were prepared one day before. Briefly, irradiated feeding cells in B cell culture media with a proprietary growth factor cocktail were dispensed into 96-well culture plates (120 μL/well). On the day of sorting, suspended and loosely attached splenocytes were collected by gently pipetting medium against the culturing surface of flask. The cells were then transferred to conical tube and spin at 400×g for 3 minutes. The cell pellets were washed with ice-cold FACS buffer (1×PBS+0.5% BSA) once. Then the biotinylated antigen was added at 5 μg/ml (final concentration). The mixture was incubated at R.T, for 20 min. The staining mixture was then centrifuged at 400×g for 3 min, and the cells were washed once in FACS buffer before being resuspended in FACS buffer and transferred into 1.5 ml amber Eppendorf tube. NeutrAvidin-Dy594 (1:300 dilution, Invitrogen cat #22842) and FITC-conjugated anti-rabbit IgM antibody (1:500 dilution, Novus, cat #MB7173) was then added, and the mixture was incubated at 4° C. for 15-30 min. The staining mixture was centrifuged at 400×g for 3 min. The cells were washed twice with ice-cold FACS buffer. The washed cell pellets were resuspended at ˜10⁷ cells/ml in ice-cold 1×PBS+1% FBS. At least 10 minutes before sorting, 7-AAD (1 g/ml, final concentration) was added for live/dead cell determination. Single 7-AAD-/FITC-/Dy594+ cell was sorted into each well of a seeded 96-well plate. 96-well B cell culture plates with sorted B cells were cultured in 37° C. with 5% CO₂ for 9-12 days.

### Heavy-Chain and Light-Chain Variable Region (V.H. And V.L.) and Linier Expression Module (LEM) Construction and mAb Expression

Positive clones from B cell sorting supernatant screening were selected for V.H. and V.L, amplification by PCR, and LEM construction according to in-house SOP (Yurogen, Worcester, MA). Total RNAs from selected clones were purified from cell pellets preserved in DNA/RNA shield using RNeasy Mini Kit (Zvmo, Cat #: R1051) following the manufacturer's protocol. Thirty-six dl nuclease-free water (Ambion, cat # AM9937) was used to elute total RNA. Eleven μl total RNA from each clone were mixed with 1 μl oligo (dT)12-18 primer (Invitrogen, Cat #58862) and 1 μl dNTPs (10 mM, ThermoScientific, cat #R0182) and then were heated at 65° C. for 5 min. Then for each clone mixture. 4 μl 5×First Strand buffer, 1 μl 100 mM DTT. 1 μl RNaseOUT (Invitrogen, Cat #: 10777-019), and 1 μl SuperScript III reverse transcriptase (Invitrogen, Cat #18080-044) was added. The reverse transcription reaction was carried out at 50° C. 1 hr and 75° C. 15 min to inactivate SuperScript III enzyme. After cDNA synthesis, V.H. and V.L. genes were amplified separately with V.H. and V.L. variable region primer pairs. The V.H. and VL PCR products were separated on 10% agarose gel electrophoresis system. The expected size of V.H. and V.L, amplicon is ˜500 bp. The corresponding bands of V.H. and V.L, for each clone were cut from gel, and V.H. and V.L. genes were extracted from gel with NucleoSpin® Gel and PCR Clean-Up Kit (Macherey-Nagel. Cat #740609.250) following manufacturer's protocol. Twelve-30 μl of elution buffer were used to elute V.H. and VL PCR products, depending on the amount of PCR products. The linear expression module cassette (LEM) PCR products were constructed by overlapping PCR with the C fragment containing CMV promotor, V.H. or V.L., and the H fragment contained rabbit IgG heavy-chain CH1-CH2-CH3 fragment, or light-chain constant region followed by SV40 transcription terminator and poly A signal sequences. Five 5 μl of PCR product were used to check the size and magnitude of amplification by 0.8% agarose gel electrophoresis. The remaining PCR products were purified with NucleoSpin® Gel and PCR Clean-Up Kit following manufacturer's protocol. Thirty-two μl of elution buffer was used to elute LEM PCR products. The LEM PCR products were transfected with 293E cells and collected supernatant post 4 day transfection for mAb screening.

### Cloning of V.H. And V.L. Into Expression Vector and Small Scale mAb Purification

V.H. and V.L. of selected clones were then cloned into the original expression vector pYURK using a one-step ligation independent cloning method. Plasmids with inserts were analyzed DNA sequences by Sanger sequencing methods. When multiple heavy or light chain constructs with different sequences were obtained for certain clones, transient expression of antibodies was performed using combinations of heavy and light chain constructs, and antigen-binding was confirmed for these supernatants by ELISA. Full-length IgG heavy and light chain sequences (including signal peptides) were obtained from functional IgG heavy and light chain plasmids' sequences. IgG heavy and light chain DNA sequences with a signal peptide for secretion were cloned into pcDNA3.4 vector which was used for Expi293F cell transfection. Cell culture supernatant was collected, and antibodies in culture supernatant were affinity purified with Protein A agarose.

### Luminex Assay

The Luminex assay was conducted using FlexMap 3D (Luminex, Austin, TX, USA), and the conjugation of VLP was previously reported [13]. Briefly, 65 μg DENV proteins (Table 17) were conjugated to 1-Ethyl-3-[3-dimethylaminopropyl]carbodiimide hydrochloride, ECD/N-hydroxy-sulfo-succinimide, NHS (Thermo Fisher, Waltham, MA, USA) activated 12.5 million MagPlex beads (Luminex, Austin, TX, USA) at 50 mM 2-(N-morpholino) ethanesulfonic acid buffer pH 5.0-7.0 for 120 minutes at room temperature.

TABLE 17
VLP and Proteins for Luminex assay

| Description | Source | Strain info | Accession No |
|---|---|---|---|
| DENV-1 VLP | Native Antigen | Puerto Rico/US/BID-V853/1998 | ABO45246.1 |
| DENV-2 VLP | Native Antigen | Thailand/16681/84 | AAP06254.1 |
| DENV-3 VLP | Native Antigen | Sri Lanka D3/H/IMTSSA- | AXX75610.1 |
| | | SRI/2000/1266 | |
| DENV-4 VLP | Native Antigen | Dominica/814669/1981 | P09866.2 |
| ZIKV VLP | Native Antigen | Suriname Z1106033 | ALX35659.1 |
| Chikungunya virus VLP, | Native Antigen | Senegal 37997 | Q5XXP3.1 |
| Dengue Virus Serotype 2 | Native Antigen | Thailand/16681/84 | AAB58782.1 |
| envelope protein | | | |
| Dengue Virus Serotype 3 | Native Antigen | Sri Lanka D3/H/IMTSSA- | AXX75610.1 |
| envelope protein | | SRI/2000/1266 | |
| Dengue Virus Serotype 4 | Native Antigen | Philippines/H241 | ALB78116.1 |
| envelope protein | | | |
| Japanese Encephalitis Virus | Native Antigen | SA-14 | P27395.1 |
| VLP | | | |
| West Nile Virus envelope | Native Antigen | NY99 | ADD23575.1 |
| protein, | | | |

After conjugation, excess active residues were blocked by Sample buffer (1% bovine serum albumin (BSA) in Dulbecco's phosphate-buffered saline, D-PBS) overnight at 4° C. 10,000 DENV proteins conjugated beads/mL and anti-DENV mAb or anti-DENV mAb expressed supernatants were incubated at room temperature in sample buffer for 90 minutes and washed with phosphate-buffered saline plus 0.05% Tween-20 (PBST). After washing, the beads were incubated 10 μg/mL of Phycoerythrin-labeled anti-rabbit IgG (Thermo Fisher, Waltham, MA, USA) for 60 minutes. The beads were washed and mixed with Sheath Fluid (Luminex, Austin, TX, USA). The plates were measured the fluorescence intensity by FlexMap 3D.

k_{off} Ranking

The dissociation rate constant (k_{off}) of candidates' mAb bound to DENV-VLP was measured using Bio-layer interferometry (BLI) using an Octet-HTX (Sartorius, Fremont, CA, USA). Briefly, antibody expressed supernatants were diluted with running buffer (0.1% Bovine Serum Albumin (BSA), PBS 0.05% Tween 20 (PBS-T)), and rabbit IgG was captured by Protein A biosensor (Sartorius, Fremont, CA, USA). The biosensors were transferred to 5 μg/mL of DENV-VLP solution for association (10 min) and then to a running buffer for dissociation. k_{off} of each mAbs was calculated by Octet Data Analysis Software H.T. (ver. 11.1.2.48 Sartorius, Fremont, CA, USA) with the Langmuir 1:1 binding model. The k_{off} or some serum samples could not be measured due to strong binding, in this case k_{off} were extrapolated to less than 2×10⁻⁵ (detectable dissociation from 0-1200 seconds for 5% signal decrease).

### Western Blot Analysis

Western blot analysis was conducted by a capillary-based electrophoresis system [14](Wes, ProteinSimple, Santa Clara, CA, USA). In brief, DENV-VLP were denatured at 70° C. without reducing agent for 5 minutes, and VLP solution was loaded on a Wes assay plate and electrophoresed. Next, 10 μg/mL of Anti-DENV mAb were charged, followed by Wes horseradish peroxidase-conjugated anti-rabbit secondary antibody. The sample run was analyzed by examining the electropherogram and digital gel image.

### Reporter Virus-Particle (RVP) Assay

RVP assay was measured following the protocol from Bohning et al. [15]. Briefly, anti-DENV mAb expressed cell medium or serial diluted mAb solution were mixed with DENV RVP and the plate was incubated at 37° C. for 60 min in a 5% CO₂ humidified incubator. 4000-7500 cells/well Assay-Ready Raji DC-SIGN Cells (Accellerate, Hamburg. Germany) were added to the 384-well plates mAb/RVP mixture and incubated at 37° C. for 72 hrs in a 5% CO₂ incubator. Cell numbers were optimized following the manufacturer's instructions. To detect the Renilla luciferase activity in the cells, the plates were equilibrated to room temperature for 15 mm and then incubated with Renilla-Glo™ Luciferase reagent according to the manufacturer's instructions (Promega, Madison, WI USA). Chemiluminescence were read with an EnSpire chemiluminescence reader (Perkin Elmer, Waltham MA USA).

### Construction of Anti-DENV mAb Expression Vectors

Each full length heavy-chain and light-chain DNA of anti-DENV mAb with a signal peptide were was synthesized and inserted to pcDNA3.4 mammalian expression vector. These antibody expression vectors were transformed to E. coli and amplified plasmid DNA was extracted, purified and sterilized for subsequent mammalian cell expression.

Antibody Expression and Purification of Anti-DENV mAbs

The light and heavy chain of rabbit mAb mammalian expression plasmids were co-transfected into Expi 293 cell systems (Thermo Fisher, Waltham, MA. USA) [16], and the transfected medium was harvested five days after transfection with centrifuging. Monoclonal antibodies were purified through rProtein A Sepharose (Cytiva, Marlborough, MA, USA). The eluted antibody was exchanged to Dulbecco's phosphate-buffered saline, D-PBS (Gibco, Waltham, MA, USA), using Amicon Ultra (Merck Millipore, Burlington, MA, USA). Antibody purity was determined by SDS-PAGE (NuPAGE, Thermo Fisher, Waltham, MA, USA) with heat-denatured, 70° C. for 10 min with reduced agents. Band intensity of SDS-PAGE was calculated by ChemoDoc Touch imaging system (BioRad, Hercules, CA USA).

### Antibody Expression Level Measurement

The antibody expression level was measured using Bio-layer interferometry (BLI) using an Octet-HTX (Sartorius, Fremont, CA, USA). Briefly, antibody expressed supernatants and rabbit polyclonal antibody (Jackson ImmunoResearch Laboratories, West Grove. PA, USA) were diluted with sample buffer (0.1% Bovine Serum Albumin (BSA), PBS 0.05% Tween 20 (PBS-T)), then these solutions were captured by Protein G biosensor (Sartorius, Fremont, CA, USA). The IgG concentration was calculated using rabbit IgG as a standard by Octet Data Analysis Software H.T. (ver. 11.1.2.48 Sartorius, Fremont, CA, USA).

### Allele Analysis

Anti-DENV mAb allele and CDR3 regions were analyzed by IMGT/V-QUEST (http://www.imgt.org/IMGT_vquest/analysis) or NCBI IGBLAST (https://www.ncbi.nlm.nih.gov/igblast)

### Epitope Binning

Epitope binning was conducted by Octet-HTX (Forte Bio Fremont, CA, USA). Briefly, anti-DENV mAbs (20 μg/mL) were captured by ECD/Sulfo-NHS activated Amine Reactive Second Generation AR2Gbiosensor (Forte Bio Fremont. CA, USA). Next, 10 μg/mL anti-DENV mAbs were preincubated with 0.5 or 3 g/mL DENV-VLP at room temperature for 10 min, and this mixture was bound to the antibody captured on the biosensor surface. These response data were subtracted by anti-DENV mAb only data. Binding activities were normalized to the response for DENV-VLP. Response signals of each pair were used for hierarchical clustering (Ward Method) using SAS JMP 13.1.0 (SAS, Cary, NC, USA).

Domain III Binding to Anti-Dengue mAbs

Anti-DENV2 and 4 mAb was diluted to 20 μg/mL 10 mM Acetate buffer pH5.0 or 4.0 (Sartorius), then 200 μL/well of antibody solution were transferred to 96 well black plates (Griner) then coupled with AR2G biosensor (Sartorius) with EDC (1-Ethyl-3-[3-dimethylaminopropyl]carbodiimide hydrochloride) and S-NHS (N-hydroxysulfosuccinimide). The coupling biosensor was blocked by 200 μL/well of IM Ethanol Amine solution pH8.5 (Sartorius), then adjusted the baseline with 200 μL/well sample buffer (0.1% BSA PBS 0.05% Tween20). 200 μL/well of 20 μg/mL DENV2 and 4 Domain III protein (Table 18) in sample buffer was incubated with an antibody-conjugated biosensor for 1200 sec. The response values at 1200 sec associated with Domain III protein were measured. The assay was done by Octet HTX (Sartorius) at 30° C.

TABLE 18
Dengue Envelope protein domain III

| Description | Source | Strain info | Accession No |
|---|---|---|---|
| DENV-1 E protein Domain III | In-house prep | Thailand/16007/1964 | AAF59976.1 |
| DENV-2 E protein Domain III | In-house prep | Thailand/16681/1984 | AAA73185.1 |
| DENV-3 E protein Domain III | In-house prep | Philippines/16562/1964 | ANG57778.1 |
| DENV-4 E protein Domain III | In-house prep | Indonesia/1036/1976 | AAB70680 |

### Data Analysis

EC₅₀ value of Luminex assay and IC₅₀ value for RVP assay were analyzed using GraphPad Prism (Ver.8.0.0. San Diego. CA USA).

### Results

Anti-DENV mAbs Screening from Rabbit

Anti-DENV2 and 4serotype-specific mAbs were selected from 1920 or 2880 well B cell sorting samples. Luminex assay and RVP assay were applied for B cell supernatant to select mAbs. For LEM products, high antigen reactivity and neutralization activity antibodies were selected by Luminex, RVP assay, and k_{off} ranking. Finally, anti-DENV antibodies with unique amino acid sequences have been chosen. The summary of the selection mAbs is shown in Table 19. Seven mAbs for DENV-2 type specific. 14 for DENV-4 type specific and 7 DENV cross reactive mAbs were selected.

TABLE 19
Summary of anti-DENV mAb screening

| | |
|---|---|
| DENV2 | DENV4 |

| | | | | |
|---|---|---|---|---|
| Specificity | CR | TS | CR | TS |

| | | |
|---|---|---|
| B cell sorting (N) | 2880 | 1920 |

| | | | | |
|---|---|---|---|---|
| B cell sorting positive clone (N) | 23 | 22 | 15 | 30 |
| LEM expression positive clone (N)* | 8 | 7 | 4 | 18 |
| Final positive clones (N) | 6 | 7 | 1 | 14 |

C.R.: cross reactive,
T.S.: serotype-specific
*LEM positive results contained non classified specificity clones (only showing neutralizing titers), these clone were counted to CR.

### Antibody Sequence Analysis

Twelve anti-DENV-2 mAb and 15 anti-DENV-4 mAbs alleles and CDR region sequences were analyzed by IMGT/V-QUEST. Heavy chain variable (V) region alleles were selected IGHVS21*01, 39*01, 40*01, 44*01, *45*01 and 69*01. Joining (J) region allele were IGHJ2*01, 3*01, 4*01, 4*02, 6*01 and 6*02. Multiple diversity (D) region allele, IGHD1-1*01, 2-1*01, 3-1*01, 4-2*01, 6-1*01, 7-1*01 and 8-1*01 were selected (Table 20 and 21). Light chain V region allele were IGKV1S1*01, 2*01, 2*02, 4*01, 10*01, 15*01, 17*01, 18*01, 19*01, 32*01, 36*01, 37*01, 42*01, 55*01, and 67*01 All J region allele was IGKJ1-2*01 (Table 22 and 23).

TABLE 20
Heavy chain allele gene analysis of anti-DENV2 mAbs

| Clone No | V Region | J Region | D Region |
|---|---|---|---|
| DENV2_1B10_H | IGHV1S21*01 | IGHJ4*01 | IGHD1-1*01 |
| DENV2_1C1_H | IGHV1S69*01 | IGHJ4*02 | IGHD7-1*01 |
| DENV2_1F4_H1 | IGHV1S40*01 | IGHJ4*01 | IGHD6-1*01 |
| DENV2_1F4_H2 | IGHV1S40*01 | IGHJ4*01 | IGHD6-1*01 |
| DENV2_3F10_H | IGHV1S69*01 | IGHJ4*02 | IGHD8-1*01 |
| DENV2_3H11_H | IGHV1S69*01 | IGHJ4*02 | IGHD3-1*01 |
| DENV2_5B9_H | IGHV1S45*01 | IGHJ4*01 | IGHD6-1*01 |
| DENV2_5E9_H | IGHV1S69*01 | IGHJ4*02 | IGHD7-1*01 |
| DENV2_5F4_H | IGHV1S69*01 | IGHJ2*01 | IGHD7-1*01 |
| DENV2_7G10_H | IGHV1S40*01 | IGHJ3*01 | IGHD1-1*01 |
| DENV2_8D3_H | IGHV1S39*01 | IGHJ2*01 | IGHD1-1*01 |
| DENV2_8D8_H | IGHV1S44*01 | IGHJ2*01 | IGHD7-1*01 |
| DENV2_9G11_H | IGHV1S69*01 | IGHJ6*01 | IGHD4-2*01 |

TABLE 21
Heavy chain allele gene analysis of anti-DENV4 mAbs

| Clone No | V Region | J Region | D Region |
|---|---|---|---|
| DENV4_1F3 | IGHV1S69*01 | IGHJ6*02 | IGHD2-1*01 |
| DENV4_1F10 | IGHV1S69*01 | IGHJ4*02 | IGHD4-2*01 |
| DENV4_3F6 | IGHVS44*01 | IGHJ6*02 | IGHD1-1*01 |
| DENV4_4F5 | IGHV1S69*01 | IGHJ4*02 | IGHD6-1*01 |
| DENV4_5B11 | IGHV1S69*01 | IGHJ2*01 | IGHD6-1*01 |
| DENV4_7D5 | IGHV1S69*01 | IGHJ4*02 | IGHD3-1*01 |
| DENV4_7F2 | IGHV1S69*01 | IGHJ2*01 | IGHD1-1*01 |
| DENV4_7G4 | IGHV1S69*01 | IGHJ2*01 | IGHD8-1*01 |
| DENV4_7G5 | IGHV1S40*01 | IGHJ4*01 | IGHD2-1*01 |
| DENV4_8C7 | IGHV1S69*01 | IGHJ4*02 | IGHD1-1*01 |
| DENV4_8C10 | IGHV1S69*01 | IGHJ4*02 | IGHD2-1*01 |
| DENV4_8G6 | IGHV1S69*01 | IGHJ4*02 | IGHD2-1*01 |
| DENV4_9F10 | IGHV1S69*01 | IGHJ4*02 | IGHD8-1*01 |
| DENV4_9G8 | IGHV1S40*01 | IGHJ4*01 | IGHD6-1*01 |
| DENV4_10A11 | IGHV1S69*01 | IGHJ2*01 | IGHD1-1*01 |

TABLE 22
Light chain allele gene analysis of anti-DENV2 mAbs

| Clone No | V Region | J Region |
|---|---|---|
| DENV2_1B10_L | IGKV1S15*01 | IGKJ1-2*01 |
| DENV2_1C1_L | IGKV1S10*01 | IGKJ1-2*01 |
| DENV2_1F4_L | IGKV1S55*01 | IGKJ1-2*01 |
| DENV2_3F10_L | IGKV1S42*01 | IGKJ1-2*01 |
| DENV2_3H11_L | IGKV1S2*01 | IGKJ1-2*01 |
| DENV2_5B9_L | IGKV1S10*01 | IGKJ1-2*01 |
| DENV2_5E9_L | IGKV1S17*01 | IGKJ1-2*01 |
| DENV2_5F4_L | IGKV1S19*01 | IGKJ1-2*01 |
| DENV2_7G10_L | IGKV1S15*01 | IGKJ1-2*01 |
| DENV2_8D3_L | IGKV1S2*01 | IGKJ1-2*01 |
| DENV2_8D8_L | IGKV1S19*01 | IGKJ1-2*01 |
| DENV2_9G11_L | IGKV1S36*01 | IGKJ1-2*01 |

TABLE 23
Light chain allele gene analysis of anti-DENV4 mAbs

| Clone No | V Region | J Region |
|---|---|---|
| DENV4_1F3_L | IGKV1S17*01 | IGKJ1-2*01 |
| DENV4_1F10_L | IGKV1S4*01 | IGKJ1-2*01 |
| DENV4_3F6_L | IGKV1S36*01 | IGKJ1-2*01 |
| DENV4_4F5_L | IGKV1S1*01 | IGKJ1-2*01 |
| DENV4_5B11_L | IGKV1S67*01 | IGKJ1-2*01 |
| DENV4_7D5_L | IGKV1S2*02 | IGKJ1-2*01 |
| DENV4_7F2_L | IGKV1S32*01 | IGKJ1-2*01 |
| DENV4_7G4_L | IGKV1S37*01 | IGKJ1-2*01 |
| DENV4_7G5_L | IGKV1S4*01 | IGKJ1-2*01 |
| DENV4_8C7_L | IGKV1S2*02 | IGKJ1-2*01 |
| DENV4_8C10_L | IGKV1S2*02 | IGKJ1-2*01 |
| DENV4_8G6_L | IGKV1S18*01 | IGKJ1-2*01 |
| DENV4_9F10_L | IGKV1S17*01 | IGKJ1-2*01 |
| DENV4_9G8_L | IGKV1S42*01 | IGKJ1-2*01 |
| DENV4_10A11_L | IGKV1S17*01 | IGKJ1-2*01 |

These antibodies showed unique CDR sequences.

Heavy chain CDR3 lengths were from 11 to 19 amino acid residues, and light chain CDR3 lengths were 10⁻¹⁵ amino acid residues.

### Antibody Expression and Purification

Twelve anti-DENV-2 mAb and 15 anti-DENV-4 mAbs were expressed using Expi293 T expression system. Most antibody clones showed high expression levels from 30.8 to 850 mg/L. (Table 19).

TABLE 24
Summary of anti-DENV2 and 4 mAb expression levels

| Clones | IgG mg/L |
|---|---|
| DENV2-1B10 | 471.0 |
| DENV2-1C1 | 546.6 |
| DENV2-1F4-H1 | 167.6 |
| DENV2-1H4_H2 | 452.9 |
| DENV2-3F10 | 649.9 |
| DENV2-3H11 | 623.4 |
| DENV2-5B9 | 30.8 |
| DENV2-5B9 | 617.3 |
| DENV2-5E9 | 712.7 |
| DENV2-5F4 | 842.7 |
| DENV2-8D8 | 429.9 |
| DENV2-7G10 | 504.9 |
| DENV2-8D3 | 318.6 |
| DENV2-8D8 | 447.0 |
| DENV2-9G11 | 528.4 |
| DENV4-1F3 | 319.7 |
| DENV4-1F10 | 86.01 |
| DENV4-3F6 | 410.5 |
| DENV4-4F5 | 360.1 |
| DENV4-5B11 | 850.0 |
| DENV4-7D5 | 510.1 |
| DENV4-7F2 | 784.6 |
| DENV4-7G4 | 355.6 |
| DENV4-7G5 | 319.6 |
| DENV4-8C7 | 367.0 |
| DENV4-8C10 | 276.1 |
| DENV4-8G6 | 394.0 |
| DENV4-9F10 | 255.3 |
| DENV4-9G8 | 263.0 |
| DENV4-10A11 | 419.7 |
| Expi293Medium | 0.8 |

These 13 anti-DENV2 mAbs and 15 anti-DENV4 mAbs were purified using rProtein A Sepharose. The purity of these mAbs was 100% confirmed by SDS-PAGE analysis.

### Antibody Binding to Dengue VLPs

The binding activity of anti-DENV2 and 4 mAb was measured by Luminex. Seven mAb and 14 mAbs showed specific binding to DENV2 and 4 VLP, and 7 mAbs showed cross react to DENV serotypes and EC₅₀ values ranged from to 9.5-11210 ng/mL. The EC₅₀ value of the clones DENV2-3H11, 5F4, 8D8, 8D3, 9G11, DENV4-1F3, 3F6, 4F5, 5B11, 7D5, 7G4, 8C7, 8C10, 9G8, and 10A1l was smaller than the EC₅₀ value of the commercial clone mAbs. (FIGS. 13 and 14, Table 25 and 26)

TABLE 25
Summary of anti-DENV2 mAb reactivity

| | | | | DENV2 | | | | |
|---|---|---|---|---|---|---|---|---|
| | | | | VLP EC50 | RVP assay | Epitope bin | Domain III | |
| No | clone | Sero specificty | Binding type | ng/mL | IC50 nM | group | binding | WES reactivity |
| 1 | DENV2-1B10 | DENV2 | VLP < E-protein | 4299.0 | ND | I | ND | |
| 2 | DENV2-1C1 | DENV2 | VLP < E-protein | 1425.0 | ND | V | ND | |
| 3 | DENV2-1F4_H1 | CR | VLP | 4070.0 | ND | NA | ND | |
| 4 | DENV2-1F4_H2 | CR | VLP | 9693.0 | ND | NA | ND | |
| 5 | DENV2-3F10 | DENV2, 3, 1 | VLP | 49.1 | ND | IV | ND | + |
| 6 | DENV2-3H11 | DENV2 | VLP < E-protein | 35.4 | 1.3 | II | ND | |
| 7 | DENV2-5B9 | DENV2 | VLP < E-protein | 193.0 | 11.2 | IV | ND | |
| 8 | DENV2-5E9 | DENV2 | E-protein < VLP | 11209.0 | 361.0 | IV | ND | ++ |
| 9 | DENV2-5F4 | DENV2 | VLP | 9.5 | ND | II | ND | |
| 10 | DENV2-8D8 | DENV2 | VLP | 11.0 | ND | II | ND | |
| 11 | DENV2-7G10 | CR | E-protein < VLP | 15174.0 | 102.3 | I | ND | |
| 12 | DENV2-8D3 | CR | VLP | 33.1 | ND | III | ND | |
| 13 | DENV2-9G11 | CR | VLP | 10.7 | ND | I | ND | |
| 14 | DV2-3H5 | DENV2 | VLP = E-protein | 74.86 | 0.2 | IV | DIII | |
| 15 | DV10 | CR | VLP = E-protein | 39.53 | NA | VI | DIII | +++ |

ND: not detected,
NA: not tested,
CR: Cross Reactive

TABLE 26
Summary of anti-DENV4 mAb reactivity

| | | | | DENV4 | | | |
|---|---|---|---|---|---|---|---|
| | | | | VLP EC50 | Epitope bin | Domain III | |
| No | clone | Sero Specificity | Binding type | ng/mL | group | binding | WES reactivity |
| 1 | DENV4-1F3 | CR | VLP = E-protein | 51.9 | I | ND | + |
| 2 | DENV4-1F10 | DENV4 | VLP | 1458.0 | I | ND | |
| 3 | DENV4-3F6 | DENV4 | VLP < E-protein | 57.6 | II | ND | |
| 4 | DENV4-4F5 | DENV4 | VLP < E-protein | 30.4 | IV | DIII | ++ |
| 5 | DENV4-5B11 | DENV4 | VLP | 19.2 | IV | ND | |
| 6 | DENV4-7D5 | DENV4 | VLP < E-protein | 23.2 | III | ND | |
| 7 | DENV4-7F2 | DENV4 | VLP < E-protein | 40.7 | III | ND | |
| 8 | DENV4-7G4 | DENV4 | VLP | 21.4 | IV | ND | |
| 9 | DENV4-7G5 | DENV4 | VLP | 20692.0 | NA | ND | |
| 10 | DENV4-8C7 | DENV4 | VLP < E-protein | 17.6 | III | ND | |
| 11 | DENV4-8C10 | DENV4 | VLP | 49.4 | III | ND | |
| 12 | DENV4-8G6 | DENV4 | VLP | 3706.0 | III | ND | |
| 13 | DENV4-9F10 | DENV4 | VLP = E-protein | 1410.0 | I | DIII | |
| 14 | DENV4-9G8 | DENV4 | VLP < E-protein | 71.3 | II | DIII | +++ |
| 15 | DENV4-10A11 | DENV4 | VLP < E-protein | 23.6 | III | ND | |
| 17 | 777-4 | DENV4 | E-protein < VLP | 187.6 | II | ND | |
| 18 | DV22 | DENV4 | E-protein < VLP | 3788.0 | VI | ND | ++ |

ND: not detected,
NA: not tested,
CR: Cross Reactive

### Western Reactivity

Five clones reacted to Western analysis. DENV2-3F10 and DENV2-5F9 detected 16 ng and 3.2 ng DFNV2 VLP/Lane. The sensitivity was almost the same as DV10. DFNV4-1F74, DENV4-4F5 and DENV4-9G8 detected 3.2 ng, 3.2 ng and 0.6 ng DENV4 VLP/Lane (FIG. 15, 16, and Table 20, 21). Commercial mAb, DV22 also detected 3.2 ng DENV4 VLP.

Neutralizing Activity of Anti-DENV2 mAbs

The neutralizing antibody activity was accessed by RVP assays. DENV2-3H111 showed strong neutralizing activity. (FIG. 17 and Table 20).

Epitope Binning of Anti-DENV2 and Anti-DENV4 mAbs

Epitope binning analysis was conducted with 11 in-house prepared mAbs and 9 commercial mAbs for DENV2, and 14 in-house prep mAbs and 11 commercial mAbs for DENV4. These anti-DENV2 and 4 mAbs were divided into 6 epitope bins, respectively (FIGS. 18 and 19).

k_{off} Ranking

The results of the k_{off} measurements for the different clones are shown in Table 27 and 28:

TABLE 27
k_{off} ranking of anti-DENV-2 antibody

| Clone | Response (nm) | koff (1/s) |
|---|---|---|
| DENV2-1B10 | 0.449 | <2.0E−05 |
| DENV2-1C1 | 0.335 | <2.0E−05 |
| DENV2-1F4 | 0.896 | <2.0E−05 |
| DENV2-3F10 | 1.315 | <2.0E−05 |
| DENV2-3H11 | 2.017 | <2.0E−05 |
| DENV2-5B9 | 0.017 | 7.75E−04 |
| DENV2-5E9 | 0.096 | 8.02E−05 |
| DENV2-5F4 | 1.499 | <2.0E−05 |
| DENV2-8D8 | 0.756 | <2.0E−05 |
| DENV2-7G10 | 1.811 | <2.0E−05 |
| DENV2-8D3 | 1.690 | <2.0E−05 |
| DENV2-9G11 | 2.556 | <2.0E−05 |

TABLE 28
k_{off} ranking of anti-DENV-4 antibody

| Clone | Response (nm) | koff (1/s) |
|---|---|---|
| DENV4-1F3 | 1.1779 | <2.0E−05 |
| DENV4-1F10 | 2.1809 | <2.0E−05 |
| DENV4-3F6 | 0.7048 | <2.0E−05 |
| DENV4-4F5 | 2.2768 | 2.85E−05 |
| DENV4-5B11 | 2.3901 | 2.36E−05 |
| DENV4-7D5 | 2.4484 | 3.56E−05 |
| DENV4-7F2 | 2.3192 | 3.67E−05 |
| DENV4-7G4 | 2.1637 | <2.0E−05 |
| DENV4-7G5 | 0.185 | 4.19E−05 |
| DENV4-8C7 | 2.3806 | 3.32E−05 |
| DENV4-8C10 | 1.6058 | 2.89E−05 |
| DENV4-8G6 | 0.3009 | <2.0E−05 |
| DENV4-9F10 | 0.1906 | 4.47E−05 |
| DENV4-9G8 | 0.1366 | <2.0E−05 |
| DENV4-10A11 | 2.169 | 4.17E−05 |

All of the antibodies according to the present invention exhibit a k_{off} rate which is less than the rate of the commercial antibody 4G2 [8].

Domain III Binding to Anti-Dengue 2 and 4 mAbs

DENV4-9F10 and 9G8 observed a high Domain III binding response. Since DV10, mAb513 confirmed the domain III binding, thus we concluded DENV4-9F10 and 9G8 binds to Domain III (Table 29 and 30).

TABLE 29
Domain III binding to anti-Dengue 2 mAbs

| | Binding | TDV2 DIII | Response |
|---|---|---|---|
| clone | domain | conc ug/mL | (nm) |
| DENV2-1B10 | NA | 20 | 0.0886 |
| DENV2-1C1 | NA | 20 | 0.0176 |
| DENV1-1F4-H1 | NA | 20 | −3.11E−03 |
| DENV2-1F4-H2 | NA | 20 | 0.0196 |
| DENV2-3F10 | NA | 20 | 0.0248 |
| DENV2-3H11 | NA | 20 | −4.52E−03 |
| DENV2-5B9 | NA | 20 | 0.0218 |
| DENV2-5E9 | NA | 20 | 0.0171 |
| DENV2-5F4 | NA | 20 | 0.0206 |
| DENV2-8D8 | NA | 20 | 0.0246 |
| DENV2-7G10 | NA | 20 | 0.0416 |
| DENV2-8D3 | NA | 20 | 0.0387 |
| DENV2-9G11 | NA | 20 | 0.0247 |
| 3H5 | DIII | 20 | 0.4883 |
| DV2-3H5 | DIII | 20 | 0.3028 |
| DV10 | DIII | 20 | 0.3456 |
| DV78 | DI/II | 20 | 0.0461 |
| DV18 | DI/II | 20 | 0.0506 |
| 4G2 | Fusion Loop | 20 | 0.0174 |
| Mab513 | DIII | 20 | 0.3113 |
| 78-2 | Fusion Loop | 20 | −2.00E−03 |

TABLE 30
Domain III binding to anti-Dengue 4 mAbs

| | Binding | TDV4 DIII | Response |
|---|---|---|---|
| Clone | Domain | conc ug/mL | (nm) |
| DENV4-1F3 | NA | 20 | −0.005 |
| DENV4-1F10 | NA | 20 | 0.006 |
| DENV4-3F6 | NA | 20 | −0.006 |
| DENV4-4F5 | NA | 20 | 0.054 |
| DENV4-5B11 | NA | 20 | 0.020 |
| DENV4-7D5 | NA | 20 | −0.007 |
| DENV4-7F2 | NA | 20 | −0.005 |
| DENV4-7G4 | NA | 20 | −0.001 |
| DENV4-7G5 | NA | 20 | −0.018 |
| DENV4-8C7 | NA | 20 | −0.014 |
| DENV4-8C10 | NA | 20 | −0.004 |
| DENV4-8G6 | NA | 20 | −0.010 |
| DENV4-9F10 | DIII | 20 | 0.207 |
| DENV4-9G8 | DIII | 20 | 0.264 |
| DENV4-10A11 | NA | 20 | 0.012 |
| DV4-75 | DIII | 20 | 0.403 |
| DV10 | DIII | 20 | 0.243 |
| DV18 | DI/DII | 20 | 0.021 |
| 777-4 | NA | 20 | 0.026 |
| DV78 | DI/DII | 20 | 0.027 |
| 78-2 | Fusion Loop | 20 | −0.070 |
| 4G2 | Fusion Loop | 20 | 0.056 |
| Mab513 | DIII | 20 | 0.308 |
| DV22 | DI/DII | 20 | 0.054 |

1. A method for determining the infectivity of a plaque-forming virus in a sample comprising the steps of:
(a) seeding cells from a virus-susceptible cell line in an assay plate and culturing the cells for a culture period;
(b) preparing serial dilutions of the virus-containing sample;
(c) adding the serially diluted samples to the cells seeded and cultured in step (a) and incubating the cells over a first incubation period;
(d) culturing the cells of step (c) over a second incubation period;
(e) lysing the cells of step (d); and
(f) determining the presence and/or amount of a virus antigen of the virus in the lysate of step (e) in an immunoassay method, wherein the amount of the virus antigen is indicative for the infectivity of the virus in the sample.

2. The method of claim 1, wherein the plaque-forming virus is a flavivirus, preferably the flavirus is selected from Yellow fever virus, Japanese encephalitis virus, dengue virus and Zika virus, more preferably, the virus is a dengue virus selected from DENV1, DENV2, DENV 3 and DENV4.

3-5. (canceled)

6. The method according to claim 1, wherein the first incubation period in step (c) is 2 to 6 hours.

7. The method according to claim 1, wherein the second incubation period for dengue serotypes 1, 2, 3 and 4 is from about 20 to about 130 hours, preferably from about 48 to 96 hours, more preferably from about 70 to about 74 hours.

8-23. (canceled)

24. A method for determining the average maturation degree of a population of flaviviruses in a flavivirus-containing sample, wherein the method comprises:
(a) coating at least one well of an assay plate with at least one primary antibody specific for a flavivirus envelope protein (E protein) antigen;
(b) contacting the at least one primary antibody coated on the at least one well of the assay plate of step (a) with the flavivirus-containing sample to allow binding of the virus antigen to the primary antibody;
(c1) contacting the virus antigen bound to the primary antibody with (i) a secondary antibody specific for the E protein antigen, wherein the at least one secondary antibody is conjugated to a detectable label; or
(c2) contacting the virus antigen bound to the primary antibody with the (i) secondary antibody specific for the E protein antigen and further contacting the at least one secondary antibody with a tertiary antibody being specific for the constant region of the at least one secondary antibody and conjugated to a detectable label;
(d) determining the amount of the E protein antigen in the sample;
(e) coating at least one well of an assay plate with at least one primary antibody specific for a flavivirus envelope protein (E protein) antigen;
(f) contacting the at least one primary antibody coated on the at least one well of the assay plate of step (e) with the flavivirus-containing sample to allow binding of the virus antigen to the primary antibody;
(g1) contacting the virus antigen bound to the primary antibody with (ii) a secondary antibody specific for a portion of the pr domain of the flavivirus prM protein, wherein the at least one secondary antibody is conjugated to a detectable label; or
(g2) contacting the virus antigen bound to the primary antibody with (ii) a secondary antibody specific for a portion of the pr domain of the flavivirus prM protein and further contacting the at least one secondary antibody with a tertiary antibody being specific for the constant region of the at least one secondary antibody and conjugated to a detectable label;
(h) determining the amount of the prM protein in the sample; and
(i) calculating the ratio of the amount of prM protein to the amount of the E protein which is indicative for the average maturation degree of the flaviviruses in the sample.

25. The method of claim 24, wherein the detectable label is selected from an enzyme, fluorescent, chemiluminescent or an electroluminescent label, preferably an electroluminescent label, more preferably a sulfotag label.

26. The method of claim 24, wherein the at least one primary antibody present in a first well of the assay plate consists of an antibody specific for a dengue virus envelope antigen from a first dengue virus serotype, and wherein the at least one primary antibody present in a second well of the assay plate consists of an antibody specific for a dengue virus envelope antigen from a second dengue virus serotype.

27. The method of claim 24, wherein the at least one primary antibody present in a first well of the assay plate comprises a mixture of
(i) a primary antibody specific for a virus antigen from a first dengue virus serotype, wherein the primary antibody is provided at a first site of the well, and
(ii) a primary antibody specific for a virus antigen from a second dengue virus serotype, wherein the primary antibody is provided at a second site of the same well.

28. The method of claim 24, wherein the (i) secondary antibody specific for the E protein antigen is selected from clone DV-78 (2BScientific Catalog No: AB00246-10-0-BT).

29. The method of claim 24, wherein the (ii) secondary antibody specific for a portion of the pr domain of the prM protein is selected from clone 2H2 (ATCC Catalog No: HB-114) and DV 62.5 (GenScript: U043WIB080-3).

30. An antibody specific for Dengue virus serotype 2 envelope protein (DENV2 E protein) or an antigen binding fragment thereof, wherein
(i) the VH CDR1 region of the antibody or antigen binding fragment thereof is selected from the group consisting of SEQ ID NO: 106, SEQ ID NO: 109, and SEQ ID NO: 112, or a variant thereof having at least 85% identity;
(ii) the VH CDR2 region of the antibody or binding fragment thereof is selected from the group consisting of SEQ ID NO: 107, SEQ ID NO: 110, and SEQ ID NO: 113, or a variant thereof having at least 85% identity;
(iii) the VH CDR3 region of the antibody or antigen binding fragment thereof is selected from the group consisting of SEQ ID NO: 108, SEQ ID NO: 111, and SEQ ID NO 114, or a variant thereof having at least 85% identity;
(iv) the VL CDR1 region of the antibody or antigen binding fragment thereof is selected from the group consisting of SEQ ID NO: 127, SEQ ID NO: 129, and SEQ ID NO: 131, or a variant thereof having at least 82% identity;
(v) the VL CDR2 region of the antibody or antigen binding fragment thereof is selected from the group of an amino acid sequence consisting of RAS, QAS and LAS, or a variant thereof having at least 65% identity; and
(vi) the VL CDR3 region of the antibody or antigen binding fragment thereof is selected from the group consisting of SEQ ID NO: 128, SEQ ID NO: 130, and SEQ ID NO: 132, or a variant thereof having at least 85% identity,
wherein the antibody or antigen binding fragment thereon does not cross-react with other dengue serotypes other than DENV2 and has one or more of the following properties:
(1) a binding activity for DENV2-VLP calculated as EC₅₀ value of 38 ng/ml or less; and/or
(2) a k_{off} value of 1×10⁻⁴ sec⁻¹ or less.

31-34. (canceled)

35. The antibody of claim 30, wherein the antibody or antigen binding fragment thereof comprises the heavy chain CDR regions 1 to 3 having the amino acid sequences of SEQ ID Nos: 106 to 108, respectively, the light chain CDR region 1 has the amino acid sequence of SEQ ID NO: 127, the light chain CDR region 2 has the amino acid sequence RAS, and the light chain CDR region 3 has the amino acid sequence SEQ ID NO: 128.

36. The antibody of claim 30, wherein the antibody or antigen binding fragment thereof comprises the heavy chain CDR regions 1 to 3 having the amino acid sequences of SEQ ID Nos: 109 to 111, respectively, the light chain CDR region 1 has the amino acid sequence of SEQ ID NO: 129, the light chain CDR region 2 has the amino acid sequence QAS, and the light chain CDR region 3 has the amino acid sequence SEQ ID NO: 130.

37. The antibody of claim 30, wherein the antibody or antigen binding fragment thereof comprises the heavy chain CDR regions 1 to 3 having the amino acid sequences of SEQ ID Nos: 112 to 114, respectively, the light chain CDR region 1 has the amino acid sequence of SEQ ID NO: 131, the light chain CDR region 2 has the amino acid sequence LAS, and the light chain CDR region 3 has the amino acid sequence SEQ ID NO: 132.

38. The antibody of claim 35, wherein the antibody or antigen binding fragment thereof comprises in the VH domain the amino acid sequence set forth in SEQ ID NO: 145 or a derivative thereof, and in the VL domain the amino acid sequence set forth in SEQ ID NO: 148 or a derivative thereof, wherein the derivative differs from the indicated sequence by not more than five amino acid mutations.

39. The antibody of claim 36, wherein the antibody or antigen binding fragment thereof comprises in the VH domain the amino acid sequence set forth in SEQ ID NO: 146 or a derivative thereof, and in the VL domain the amino acid sequence set forth in SEQ ID NO: 149 or a derivative thereof, wherein the derivative differs from the indicated sequence by not more than five amino acid mutations.

40. The antibody of claim 37, wherein the antibody or antigen binding fragment thereof comprises in the VH domain the amino acid sequence set forth in SEQ ID NO: 147 or a derivative thereof, and in the VL domain the amino acid sequence set forth in SEQ ID NO: 150 or a derivative thereof, wherein the derivative differs from the indicated sequence by not more than five amino acid mutations.

41-44. (canceled)

45. An antibody specific for Dengue virus serotype 4 envelope protein (DENV4 E protein) or an antigen binding fragment thereof, wherein
(i) the VH CDR1 region of the antibody or antigen binding fragment thereof is selected from the group consisting of SEQ ID NO: 1, SEQ ID NO: 4, SEQ ID NO: 7, SEQ ID NO: 10, SEQ ID NO: 13, SEQ ID NO: 16 and SEQ ID NO: 19, or a variant thereof having at least 85% identity;
(ii) the VH CDR2 region of the antibody or binding fragment thereof is selected from the group consisting of SEQ ID NO: 2, SEQ ID NO: 5, SEQ ID NO: 8, SEQ ID NO: 11, SEQ ID NO: 14, SEQ ID NO: 17 and SEQ ID NO: 20, or a variant thereof having at least 85% identity;
(iii) the VH CDR3 region of the antibody or antigen binding fragment thereof is selected from the group consisting of SEQ ID NO: 3, SEQ ID NO: 6, SEQ ID NO: 9, SEQ ID NO: 12, SEQ ID NO: 15, SEQ ID NO: 18 and SEQ ID NO: 21, or a variant thereof having at least 85% identity;
(iv) the VL CDR1 region of the antibody or antigen binding fragment thereof is selected from the group consisting of SEQ ID NO: 50, SEQ ID NO: 52, SEQ ID NO: 54 and SEQ ID NO: 56, SEQ ID NO: 58, SEQ ID NO: 60 and SEQ ID NO: 62 or a variant thereof having at least 82% identity;
(v) the VL CDR2 region of the antibody or antigen binding fragment thereof is selected from the group of an amino acid sequence consisting of RAS, EAS and RAF, or a variant thereof having at least 65% identity; and
(vi) the VL CDR3 region of the antibody or antigen binding fragment thereof is selected from the group consisting of SEQ ID NO: 51, SEQ ID NO: 53, SEQ ID NO: 55 and SEQ ID NO: 57, SEQ ID NO: 59, SEQ ID NO: 61 and SEQ ID NO: 63, or a variant thereof having at least 85% identity,
wherein the antibody or antigen binding fragment thereof does not cross-react with dengue serotypes other than DENV4 and has one or more of the following properties:
(1) a binding activity for DENV4-VLP calculated as EC₅₀ value of 80 ng/ml or less; and/or
(2) a k_{off} value of 1×10⁻⁴ sec⁻¹ or less.

46-50. (canceled)

51. The antibody of claim 45, wherein the antibody or antigen binding fragment thereof comprises the heavy chain CDR regions 1 to 3 having the amino acid sequences of SEQ ID Nos: 1 to 3, respectively, the light chain CDR region 1 has the amino acid sequence of SEQ ID NO: 50, the light chain CDR region 2 has the amino acid sequence RAS, and the light chain CDR region 3 has the amino acid sequence SEQ ID NO: 51.

52. The antibody of claim 45, wherein the antibody or antigen binding fragment thereof comprises the heavy chain CDR regions 1 to 3 having the amino acid sequences of SEQ ID Nos: 4 to 6, respectively, the light chain CDR region 1 has the amino acid sequence of SEQ ID NO: 52, the light chain CDR region 2 has the amino acid sequence EAS, and the light chain CDR region 3 has the amino acid sequence SEQ ID NO: 53.

53. The antibody of claim 45, wherein the antibody or antigen binding fragment thereof comprises the heavy chain CDR regions 1 to 3 having the amino acid sequences of SEQ ID Nos: 7 to 9, respectively, the light chain CDR region 1 has the amino acid sequence of SEQ ID NO: 54, the light chain CDR region 2 has the amino acid sequence RAS, and the light chain CDR region 3 has the amino acid sequence SEQ ID NO: 55.

54. The antibody of claim 45, wherein the antibody or antigen binding fragment thereof comprises the heavy chain CDR regions 1 to 3 having the amino acid sequences of SEQ ID Nos: 10 to 12, respectively, the light chain CDR region 1 has the amino acid sequence of SEQ ID NO: 56, the light chain CDR region 2 has the amino acid sequence RAF, and the light chain CDR region 3 has the amino acid sequence SEQ ID NO: 57.

55. The antibody of claim 45, wherein the antibody or antigen binding fragment thereof comprises the heavy chain CDR regions 1 to 3 having the amino acid sequences of SEQ ID Nos: 13 to 15, respectively, the light chain CDR region 1 has the amino acid sequence of SEQ ID NO: 58, the light chain CDR region 2 has the amino acid sequence EAS, and the light chain CDR region 3 has the amino acid sequence SEQ ID NO: 59.

56. The antibody of claim 45, wherein the antibody or antigen binding fragment thereof comprises the heavy chain CDR regions 1 to 3 having the amino acid sequences of SEQ ID Nos: 16 to 18, respectively, the light chain CDR region 1 has the amino acid sequence of SEQ ID NO: 60, the light chain CDR region 2 has the amino acid sequence RAS, and the light chain CDR region 3 has the amino acid sequence SEQ ID NO: 61.

57. The antibody of claim 45, wherein the antibody or antigen binding fragment thereof comprises the heavy chain CDR regions 1 to 3 having the amino acid sequences of SEQ ID Nos: 19 to 21, respectively, the light chain CDR region 1 has the amino acid sequence of SEQ ID NO: 62, the light chain CDR region 2 has the amino acid sequence RAS, and the light chain CDR region 3 has the amino acid sequence SEQ ID NO: 63.

58. The antibody of claim 51, wherein the antibody or antigen binding fragment thereof comprises in the VH domain the amino acid sequence set forth in SEQ ID NO: 92 or a derivative thereof, and in the VL domain the amino acid sequence set forth in SEQ ID NO: 99 or a derivative thereof, wherein the derivative differs from the indicated sequence by not more than five amino acid mutations.

59. The antibody of claim 52, wherein the antibody or antigen binding fragment thereof comprises in the VH domain the amino acid sequence set forth in SEQ ID NO: 93 or a derivative thereof, and in the VL domain the amino acid sequence set forth in SEQ ID NO: 100 or a derivative thereof, wherein the derivative differs from the indicated sequence by not more than five amino acid mutations.

60. The antibody of claim 53, wherein the antibody or antigen binding fragment thereof comprises in the VH domain the amino acid sequence set forth in SEQ ID NO: 94 or a derivative thereof, and in the VL domain the amino acid sequence set forth in SEQ ID NO: 101 or a derivative thereof, wherein the derivative differs from the indicated sequence by not more than five amino acid mutations.

61. The antibody of claim 54, wherein the antibody or antigen binding fragment thereof comprises in the VH domain the amino acid sequence set forth in SEQ ID NO: 95 or a derivative thereof, and in the VL domain the amino acid sequence set forth in SEQ ID NO: 102 or a derivative thereof, wherein the derivative differs from the indicated sequence by not more than five amino acid mutations.

62. The antibody of claim 55, wherein the antibody or antigen binding fragment thereof comprises in the VH domain the amino acid sequence set forth in SEQ ID NO: 96 or a derivative thereof, and in the VL domain the amino acid sequence set forth in SEQ ID NO: 103 or a derivative thereof, wherein the derivative differs from the indicated sequence by not more than five amino acid mutations.

63. The antibody of claim 56, wherein the antibody or antigen binding fragment thereof comprises in the VH domain the amino acid sequence set forth in SEQ ID NO: 97 or a derivative thereof, and in the VL domain the amino acid sequence set forth in SEQ ID NO: 104 or a derivative thereof, wherein the derivative differs from the indicated sequence by not more than five amino acid mutations.

64. The antibody of claim 57, wherein the antibody or antigen binding fragment thereof comprises in the VH domain the amino acid sequence set forth in SEQ ID NO: 98 or a derivative thereof, and in the VL domain the amino acid sequence set forth in SEQ ID NO: 105 or a derivative thereof, wherein the derivative differs from the indicated sequence by not more than five amino acid mutations.

65-85. (canceled)


SEQUENCE LISTING (154 sequences)

SEQ ID NO: 1 - protein, 8 aa, Oryctolagus cuniculus
GFSLSDSA

SEQ ID NO: 2 - protein, 8 aa, Oryctolagus cuniculus
IYAGSGTT

SEQ ID NO: 3 - protein, 18 aa, Oryctolagus cuniculus
ARATAYVGYGYGDDTFDP

SEQ ID NO: 4 - protein, 8 aa, Oryctolagus cuniculus
GIDLSSWE

SEQ ID NO: 5 - protein, 7 aa, Oryctolagus cuniculus
IFPSGTI

SEQ ID NO: 6 - protein, 11 aa, Oryctolagus cuniculus
GRHGGSLYLNI

SEQ ID NO: 7 - protein, 8 aa, Oryctolagus cuniculus
GIDLSYYA

SEQ ID NO: 8 - protein, 7 aa, Oryctolagus cuniculus
IDTGDSA

SEQ ID NO: 9 - protein, 15 aa, Oryctolagus cuniculus
GRSLSSYYDWDAFDP

SEQ ID NO: 10 - protein, 8 aa, Oryctolagus cuniculus
GFSLSSCA

SEQ ID NO: 11 - protein, 8 aa, Oryctolagus cuniculus
IYVDSDTT

SEQ ID NO: 12 - protein, 18 aa, Oryctolagus cuniculus
ARTTAYVGGGYGDDTFDP

SEQ ID NO: 13 - protein, 8 aa, Oryctolagus cuniculus
GIDLSSYE

SEQ ID NO: 14 - protein, 7 aa, Oryctolagus cuniculus
IFPSGTI

SEQ ID NO: 15 - protein, 11 aa, Oryctolagus cuniculus
GRHGGSLYLNI

SEQ ID NO: 16 - protein, 9 aa, Oryctolagus cuniculus
GFSFSYRYW

SEQ ID NO: 17 - protein, 8 aa, Oryctolagus cuniculus
IDTGNGDT

SEQ ID NO: 18 - protein, 15 aa, Oryctolagus cuniculus
ARSDVTLTGYGYGNL

SEQ ID NO: 19 - protein, 8 aa, Oryctolagus cuniculus
GINLSFYA

SEQ ID NO: 20 - protein, 7 aa, Oryctolagus cuniculus
IDTDNSA

SEQ ID NO: 21 - protein, 15 aa, Oryctolagus cuniculus
ARSLSSFYDWDAFDP

SEQ ID NO: 22 - protein, 24 aa, Oryctolagus cuniculus
QSVEESGGRLVTPGTPLTLTCTVS

SEQ ID NO: 23 - protein, 17 aa, Oryctolagus cuniculus
MSWVRQAPGKGLEWIGY

SEQ ID NO: 24 - protein, 36 aa, Oryctolagus cuniculus
YYASWAKGRFTISKTSTTVDLKITSPTTEDTATYFC

SEQ ID NO: 25 - protein, 11 aa, Oryctolagus cuniculus
WGPGTLVTVSS

SEQ ID NO: 26 - protein, 24 aa, Oryctolagus cuniculus
QSVEESGGRLVTPGTPLTLTCTVS

SEQ ID NO: 27 - protein, 17 aa, Oryctolagus cuniculus
MGWVRQAPGKGLEWIGI

SEQ ID NO: 28 - protein, 36 aa, Oryctolagus cuniculus
YYANWAKGRFAISKASTTVDLKITSPTTEDTATYFC

SEQ ID NO: 29 - protein, 11 aa, Oryctolagus cuniculus
WGPGTLVTVSL

SEQ ID NO: 30 - protein, 24 aa, Oryctolagus cuniculus
QSVEESGGRLVTPGTPLTLTCTVS

SEQ ID NO: 31 - protein, 17 aa, Oryctolagus cuniculus
MGWVRQAPGEGLECIGF

SEQ ID NO: 32 - protein, 37 aa, Oryctolagus cuniculus
YYASWAKGRFTISKTSSTTVDLKITSPTPEDTATYFC

SEQ ID NO: 33 - protein, 11 aa, Oryctolagus cuniculus
WGPGTLVTVSS

SEQ ID NO: 34 - protein, 24 aa, Oryctolagus cuniculus
QSVEESGGRLVTPGTPLTLTCTVS

SEQ ID NO: 35 - protein, 17 aa, Oryctolagus cuniculus
MTWVRQAPGKGLEWIGY

SEQ ID NO: 36 - protein, 36 aa, Oryctolagus cuniculus
YYASWAKGRFTISTTSTTVDLRITSPTTEDTATYFC

SEQ ID NO: 37 - protein, 11 aa, Oryctolagus cuniculus
WGPGTLVTVSS

SEQ ID NO: 38 - protein, 24 aa, Oryctolagus cuniculus
QSVEESGGRLVTPGTPLTLTCTVS

SEQ ID NO: 39 - protein, 17 aa, Oryctolagus cuniculus
MGWVRQAPGKGLEWIGI

SEQ ID NO: 40 - protein, 36 aa, Oryctolagus cuniculus
YYANWAKGRFTISKTSTTVDLKITSPTTEDTATYFC

SEQ ID NO: 41 - protein, 11 aa, Oryctolagus cuniculus
WGPGTLVTVSL

SEQ ID NO: 42 - protein, 24 aa, Oryctolagus cuniculus
QSLEESGGGLVKPGASLTLTCTAS

SEQ ID NO: 43 - protein, 17 aa, Oryctolagus cuniculus
MSWVRQAPGKRPEWIAC

SEQ ID NO: 44 - protein, 37 aa, Oryctolagus cuniculus
YYASRAKGRFTIMKTSSTTVTLQMTSLTVADTATYFC

SEQ ID NO: 45 - protein, 11 aa, Oryctolagus cuniculus
WGPGTLVTVSS

SEQ ID NO: 46 - protein, 24 aa, Oryctolagus cuniculus
QSVEESGGRLVTPGTPLTLTCTVS

SEQ ID NO: 47 - protein, 17 aa, Oryctolagus cuniculus
MGWVRQAPGKGLECVGF

SEQ ID NO: 48 - protein, 36 aa, Oryctolagus cuniculus; identical to SEQ ID NO: 24

SEQ ID NO: 49 - protein, 11 aa, Oryctolagus cuniculus
WGPGTLVTVSS

SEQ ID NO: 50 - protein, 6 aa, Oryctolagus cuniculus
QSISSY

SEQ ID NO: 51 - protein, 12 aa, Oryctolagus cuniculus
QQGYSNSDVDNV

SEQ ID NO: 52 - protein, 8 aa, Oryctolagus cuniculus
QSVYVNRN

SEQ ID NO: 53 - protein, 13 aa, Oryctolagus cuniculus
QGEFGCSSADCIA

SEQ ID NO: 54 - protein, 6 aa, Oryctolagus cuniculus
QTIGNL

SEQ ID NO: 55 - protein, 12 aa, Oryctolagus cuniculus
QQGASANDIDNS

SEQ ID NO: 56 - protein, 6 aa, Oryctolagus cuniculus
QNINSY

SEQ ID NO: 57 - protein, 12 aa, Oryctolagus cuniculus
QQGYSFSGVDNV

SEQ ID NO: 58 - protein, 8 aa, Oryctolagus cuniculus
QSVYVNRN

SEQ ID NO: 59 - protein, 13 aa, Oryctolagus cuniculus
QGEFGCSSADCIA

SEQ ID NO: 60 - protein, 5 aa, Oryctolagus cuniculus
HNIYN

SEQ ID NO: 61 - protein, 12 aa, Oryctolagus cuniculus
QQYYGGDDDGFG

SEQ ID NO: 62 - protein, 6 aa, Oryctolagus cuniculus
QSISNL

SEQ ID NO: 63 - protein, 12 aa, Oryctolagus cuniculus
QQGASNSDIDNA

SEQ ID NO: 64 - protein, 26 aa, Oryctolagus cuniculus
AYDMTQTPASVEVAVGGTVTIKCQAS

SEQ ID NO: 65 - protein, 17 aa, Oryctolagus cuniculus
LSWYQQKPGQRPKLLIY

SEQ ID NO: 66 - protein, 36 aa, Oryctolagus cuniculus
TLASGVSSRFKGSGSGTEFTLTISGVQCDDAATYYC

SEQ ID NO: 67 - protein, 10 aa, Oryctolagus cuniculus
FGGGTEVVVK

SEQ ID NO: 68 - protein, 26 aa, Oryctolagus cuniculus
AQVLTQTPSPVSAAVGGTVTINCQAS

SEQ ID NO: 69 - protein, 17 aa, Oryctolagus cuniculus
LAWYQQKPGQPPKVLIY

SEQ ID NO: 70 - protein, 36 aa, Oryctolagus cuniculus
KLASGVPSRFKGSGSGTQFTLTISGVQCDDAATYYC

SEQ ID NO: 71 - protein, 10 aa, Oryctolagus cuniculus
FGGGTEVVVK

SEQ ID NO: 72 - protein, 26 aa, Oryctolagus cuniculus
AYDMTQTPASVEVAVGGTVTINCQAS

SEQ ID NO: 73 - protein, 16 aa, Oryctolagus cuniculus
LAWYQQKPGQRPKLLI

SEQ ID NO: 74 - protein, 36 aa, Oryctolagus cuniculus
TLASGVPSRFKGSGSGTQFTLTISGVECADAATYYC

SEQ ID NO: 75 - protein, 10 aa, Oryctolagus cuniculus
FGGGTAVVVK

SEQ ID NO: 76 - protein, 26 aa, Oryctolagus cuniculus
DVVMTQTPASVEAAVGGTVTINCQAS

SEQ ID NO: 77 - protein, 17 aa, Oryctolagus cuniculus
LSWYQQKPGQRPKLLIY

SEQ ID NO: 78 - protein, 36 aa, Oryctolagus cuniculus
TLASGVSSRFKGSGSGTQFTLTISGVECADAATYYC

SEQ ID NO: 79 - protein, 10 aa, Oryctolagus cuniculus
FGGGTEVVVK

SEQ ID NO: 80 - protein, 26 aa, Oryctolagus cuniculus
AQVLTQTPSPVSAAVGGTVTINCQAS

SEQ ID NO: 81 - protein, 17 aa, Oryctolagus cuniculus
LAWYQQKPGQPPKVLIY

SEQ ID NO: 82 - protein, 36 aa, Oryctolagus cuniculus; identical to SEQ ID NO: 70

SEQ ID NO: 83 - protein, 10 aa, Oryctolagus cuniculus
FGGGTEVVVK

SEQ ID NO: 84 - protein, 26 aa, Oryctolagus cuniculus
DSVMTQTPASVSEPVGGTVTINCQAS

SEQ ID NO: 85 - protein, 17 aa, Oryctolagus cuniculus
LAWYQQKPGQPPKLLIY

SEQ ID NO: 86 - protein, 36 aa, Oryctolagus cuniculus
TLESGVPSRVKGSGYGTEYTLTISDLECADAATYYC

SEQ ID NO: 87 - protein, 10 aa, Oryctolagus cuniculus
FGGGTEVVVK

SEQ ID NO: 88 - protein, 26 aa, Oryctolagus cuniculus
AYDMTQTPASVEVAVGGTVTIKCLAS

SEQ ID NO: 89 - protein, 17 aa, Oryctolagus cuniculus
LAWYQQKPGQRPKLLIY

SEQ ID NO: 90 - protein, 36 aa, Oryctolagus cuniculus; identical to SEQ ID NO: 74

SEQ ID NO: 91 - protein, 10 aa, Oryctolagus cuniculus
FGGGTEVVVK

SEQ ID NO: 92 - protein, 122 aa, Oryctolagus cuniculus
QSVEESGGRLVTPGTPLTLTCTVSGFSLSDSAMSWVRQAPGKGLEWIGYIYAGSGTTYYASWAKGRFTISKTSTTVDLKITSPTTEDTATYFCARATAYVGYGYGDDTFDPWGPGTLVTVSS

SEQ ID NO: 93 - protein, 114 aa, Oryctolagus cuniculus
QSVEESGGRLVTPGTPLTLTCTVSGIDLSSWEMGWVRQAPGKGLEWIGIIFPSGTIYYANWAKGRFAISKASTTVDLKITSPTTEDTATYFCGRHGGSLYLNIWGPGTLVTVSL

SEQ ID NO: 94 - protein, 119 aa, Oryctolagus cuniculus
QSVEESGGRLVTPGTPLTLTCTVSGIDLSYYAMGWVRQAPGEGLECIGFIDTGDSAYYASWAKGRFTISKTSSTTVDLKITSPTPEDTATYFCGRSLSSYYDWDAFDPWGPGTLVTVSS

SEQ ID NO: 95 - protein, 122 aa, Oryctolagus cuniculus
QSVEESGGRLVTPGTPLTLTCTVSGFSLSSCAMTWVRQAPGKGLEWIGYIYVDSDTTYYASWAKGRFTISTTSTTVDLRITSPTTEDTATYFCARTTAYVGGGYGDDTFDPWGPGTLVTVSS

SEQ ID NO: 96 - protein, 114 aa, Oryctolagus cuniculus
QSVEESGGRLVTPGTPLTLTCTVSGIDLSSYEMGWVRQAPGKGLEWIGIIFPSGTIYYANWAKGRFTISKTSTTVDLKITSPTTEDTATYFCGRHGGSLYLNIWGPGTLVTVSL

SEQ ID NO: 97 - protein, 121 aa, Oryctolagus cuniculus
QSLEESGGGLVKPGASLTLTCTASGFSFSYRYWMSWVRQAPGKRPEWIACIDTGNGDTYYASRAKGRFTIMKTSSTTVTLQMTSLTVADTATYFCARSDVTLTGYGYGNLWGPGTLVTVSS

SEQ ID NO: 98 - protein, 118 aa, Oryctolagus cuniculus
QSVEESGGRLVTPGTPLTLTCTVSGINLSFYAMGWVRQAPGKGLECVGFIDTDNSAYYASWAKGRFTISKTSTTVDLKITSPTTEDTATYFCARSLSSFYDWDAFDPWGPGTLVTVSS

SEQ ID NO: 99 - protein, 110 aa, Oryctolagus cuniculus
AYDMTQTPASVEVAVGGTVTIKCQASQSISSYLSWYQQKPGQRPKLLIYRASTLASGVSSRFKGSGSGTEFTLTISGVQCDDAATYYCQQGYSNSDVDNVFGGGTEVVVK

SEQ ID NO: 100 - protein, 113 aa, Oryctolagus cuniculus
AQVLTQTPSPVSAAVGGTVTINCQASQSVYVNRNLAWYQQKPGQPPKVLIYEASKLASGVPSRFKGSGSGTQFTLTISGVQCDDAATYYCQGEFGCSSADCIAFGGGTEVVVK

SEQ ID NO: 101 - protein, 110 aa, Oryctolagus cuniculus
AYDMTQTPASVEVAVGGTVTINCQASQTIGNLLAWYQQKPGQRPKLLIYRASTLASGVPSRFKGSGSGTQFTLTISGVECADAATYYCQQGASANDIDNSFGGGTAVVVK

SEQ ID NO: 102 - protein, 110 aa, Oryctolagus cuniculus
DVVMTQTPASVEAAVGGTVTINCQASQNINSYLSWYQQKPGQRPKLLIYRAFTLASGVSSRFKGSGSGTQFTLTISGVECADAATYYCQQGYSFSGVDNVFGGGTEVVVK

SEQ ID NO: 103 - protein, 113 aa, Oryctolagus cuniculus; identical to SEQ ID NO: 100

SEQ ID NO: 104 - protein, 110 aa, Oryctolagus cuniculus
DSVMTQTPASVSEPVGGTVTINCQASHNIYNSLAWYQQKPGQPPKLLIYRASTLESGVPSRVKGSGYGTEYTLTISDLECADAATYYCQQYYGGDDDGFGFGGGTEVVVK

SEQ ID NO: 105 - protein, 110 aa, Oryctolagus cuniculus
AYDMTQTPASVEVAVGGTVTIKCLASQSISNLLAWYQQKPGQRPKLLIYRASTLASGVPSRFKGSGSGTQFTLTISGVECADAATYYCQQGASNSDIDNAFGGGTEVVVK

SEQ ID NO: 106 - protein, 8 aa, Oryctolagus cuniculus
GFSLINYD

SEQ ID NO: 107 - protein, 7 aa, Oryctolagus cuniculus
VSGGGHT

SEQ ID NO: 108 - protein, 5 aa, Oryctolagus cuniculus
GRWDI

SEQ ID NO: 109 - protein, 8 aa, Oryctolagus cuniculus
GIDLSNTV

SEQ ID NO: 110 - protein, 7 aa, Oryctolagus cuniculus
ITPSGSA

SEQ ID NO: 111 - protein, 12 aa, Oryctolagus cuniculus
ARIAWSTGRNDI

SEQ ID NO: 112 - protein, 8 aa, Oryctolagus cuniculus
GIDLSSTI

SEQ ID NO: 113 - protein, 7 aa, Oryctolagus cuniculus
IGSSGSI

SEQ ID NO: 114 - protein, 12 aa, Oryctolagus cuniculus
ARIAWSTGRNDI

SEQ ID NO: 115 - protein, 24 aa, Oryctolagus cuniculus
QSVEESGGRLVTPGTPLTLTCTVS

SEQ ID NO: 116 - protein, 17 aa, Oryctolagus cuniculus
MSWIRQAPGKGLEWIGM

SEQ ID NO: 117 - protein, 37 aa, Oryctolagus cuniculus
NYASWANGRFTISKTSSTTMDLKITSPTTEDTASYFC

SEQ ID NO: 118 - protein, 11 aa, Oryctolagus cuniculus
WGPGTLVTVSL

SEQ ID NO: 119 - protein, 24 aa, Oryctolagus cuniculus
QSVEESGGRLVKPDETLTLTCTVS

SEQ ID NO: 120 - protein, 17 aa, Oryctolagus cuniculus
ISWVRQAPGKGLEYIGM

SEQ ID NO: 121 - protein, 36 aa, Oryctolagus cuniculus
YSATWAKGRVTISRTSTTVDLKIASPTTEDTATYFC

SEQ ID NO: 122 - protein, 11 aa, Oryctolagus cuniculus
WGPGTLVTVSS

SEQ ID NO: 123 - protein, 24 aa, Oryctolagus cuniculus
QSLEESGGRLVKPDETLTLTCTVS

SEQ ID NO: 124 - protein, 17 aa, Oryctolagus cuniculus
ITWVRQAPGKGLEYIGI

SEQ ID NO: 125 - protein, 36 aa, Oryctolagus cuniculus
ISANWAKGRFTISRTSTTVDLKMTSLTTEDTATYFC

SEQ ID NO: 126 - protein, 11 aa, Oryctolagus cuniculus
WGPGTLVTVSS

SEQ ID NO: 127 - protein, 8 aa, Oryctolagus cuniculus
QSVYDDNW

SEQ ID NO: 128 - protein, 11 aa, Oryctolagus cuniculus
LGGYVDDGDTT

SEQ ID NO: 129 - protein, 8 aa, Oryctolagus cuniculus
QSVYNNNA

SEQ ID NO: 130 - protein, 11 aa, Oryctolagus cuniculus
LGIYDDDVDNT

SEQ ID NO: 131 - protein, 8 aa, Oryctolagus cuniculus
QSVYNNNA

SEQ ID NO: 132 - protein, 11 aa, Oryctolagus cuniculus
LGVDDDDVDNT

SEQ ID NO: 133 - protein, 26 aa, Oryctolagus cuniculus
AVVVTQTASPVSAAVGGTVTISCQSS

SEQ ID NO: 134 - protein, 17 aa, Oryctolagus cuniculus
LAWYQQKPGQPPKQLIY

SEQ ID NO: 135 - protein, 36 aa, Oryctolagus cuniculus
RLASGVPSRFSGTGSGTQFTLTISGVQCDDAATYYC

SEQ ID NO: 136 - protein, 10 aa, Oryctolagus cuniculus
FGGGTEVVVK

SEQ ID NO: 137 - protein, 26 aa, Oryctolagus cuniculus
AAVLTQTPPSVSAAVGGTVTIKCQSS

SEQ ID NO: 138 - protein, 17 aa, Oryctolagus cuniculus
LSWYQQKPGQPPKLLIY

SEQ ID NO: 139 - protein, 36 aa, Oryctolagus cuniculus
KLSSGVSSRFSGSGSGTHFTLTISGVQCDDAATYYC

SEQ ID NO: 140 - protein, 10 aa, Oryctolagus cuniculus
FGGGTEVVVG

SEQ ID NO: 141 - protein, 26 aa, Oryctolagus cuniculus
AAVLTQTPPSVSAAVGGTVTISCQSS

SEQ ID NO: 142 - protein, 17 aa, Oryctolagus cuniculus
LSWYQQKPGQPPKLLIY

SEQ ID NO: 143 - protein, 36 aa, Oryctolagus cuniculus
NLASGVPSRFSGSGSGTQFTLTISGVQCDDAATYHC

SEQ ID NO: 144 - protein, 10 aa, Oryctolagus cuniculus
FGGGTEVVVK

SEQ ID NO: 145 - protein, 109 aa, Oryctolagus cuniculus
QSVEESGGRLVTPGTPLTLTCTVSGFSLINYDMSWIRQAPGKGLEWIGMVSGGGHTNYASWANGRFTISKTSSTTMDLKITSPTTEDTASYFCGRWDIWGPGTLVTVSL

SEQ ID NO: 146 - protein, 115 aa, Oryctolagus cuniculus
QSVEESGGRLVKPDETLTLTCTVSGIDLSNTVISWVRQAPGKGLEYIGMITPSGSAYSATWAKGRVTISRTSTTVDLKIASPTTEDTATYFCARIAWSTGRNDIWGPGTLVTVSS

SEQ ID NO: 147 - protein, 115 aa, Oryctolagus cuniculus
QSLEESGGRLVKPDETLTLTCTVSGIDLSSTIITWVRQAPGKGLEYIGIIGSSGSIISANWAKGRFTISRTSTTVDLKMTSLTTEDTATYFCARIAWSTGRNDIWGPGTLVTVSS

SEQ ID NO: 148 - protein, 111 aa, Oryctolagus cuniculus
AVVVTQTASPVSAAVGGTVTISCQSSQSVYDDNWLAWYQQKPGQPPKQLIYRASRLASGVPSRFSGTGSGTQFTLTISGVQCDDAATYYCLGGYVDDGDTTFGGGTEVVVK

SEQ ID NO: 149 - protein, 111 aa, Oryctolagus cuniculus
AAVLTQTPPSVSAAVGGTVTIKCQSSQSVYNNNALSWYQQKPGQPPKLLIYQASKLSSGVSSRFSGSGSGTHFTLTISGVQCDDAATYYCLGIYDDDVDNTFGGGTEVVVG

SEQ ID NO: 150 - protein, 111 aa, Oryctolagus cuniculus
AAVLTQTPPSVSAAVGGTVTISCQSSQSVYNNNALSWYQQKPGQPPKLLIYLASNLASGVPSRFSGSGSGTQFTLTISGVQCDDAATYHCLGVDDDDVDNTFGGGTEVVVK

SEQ ID NO: 151 - protein, 121 aa, Oryctolagus cuniculus
QSLEESGGDLVKPGASLTLTCTASGFSFSSGYDMCWVRQAPGKGLEWIACIYITDTGSSYYATWARGRFTISKTSSTSVTLQVTSLTAADTATYFCAKTNYGSGGFAFNLWGPGTLVTVSS

SEQ ID NO: 152 - protein, 111 aa, Oryctolagus cuniculus
DVVMTQTPASVEARVGGTVTINCQASQSISNLLAWYQQKPGQPPKLLIYRASALESGVPSRFRGSGSGTEFTLTISDLECADAATYYCQCTYGGSSITNWAFGGGTEVVVK

SEQ ID NO: 153 - protein, 126 aa, Oryctolagus cuniculus
QSLEESGGDLVKPGASLTLTCKASGFSFSSGSYMCWVRQAPGKGLDWIACIYSGGDVTYYTSWAKGRFTISRTSSTTVTLQMTSLTAADTATYFCARGVGTYNYAGYAYPYYFSLWGPGTLVTVSS

SEQ ID NO: 154 - protein, 110 aa, Oryctolagus cuniculus
ALVMTQTPSSVSEPVGGTVAINCQASENVYGSLAWYQQKPGQPPKLLIYGASYLASGVPSRFGGSGSGTEFTLTISDLECADAATYYCQGAYYASNFDATFGGGTEVVVK